SEQUENCE LISTING (74 sequences)

SEQ ID NO: 1 - DNA, 24 bp, Artificial Sequence
  Synthetic
tctgggcagtgacaaaaccatacc

SEQ ID NO: 2 - DNA, 22 bp, Artificial Sequence
  Synthetic
agaccacgggacctctttcact

SEQ ID NO: 3 - DNA, 23 bp, Artificial Sequence
  Synthetic
ccctgcagttttcttgcctccta

SEQ ID NO: 4 - DNA, 20 bp, Artificial Sequence
  Synthetic
ctaccctgccctgcctgaag

SEQ ID NO: 5 - DNA, 22 bp, Artificial Sequence
  Synthetic
cccaccacaaagcactgtcatg

SEQ ID NO: 6 - DNA, 24 bp, Artificial Sequence
  Synthetic
tgatcgttgtaaacagtggccttt

SEQ ID NO: 7 - DNA, 22 bp, Artificial Sequence
  Synthetic
gttggaaccacaggactggaat

SEQ ID NO: 8 - DNA, 25 bp, Artificial Sequence
  Synthetic
atggagaacctgtgattctactgaa

SEQ ID NO: 9 - DNA, 22 bp, Artificial Sequence
  Synthetic
ggggaagcaggctcagagagat

SEQ ID NO: 10 - DNA, 22 bp, Artificial Sequence
  Synthetic
gcagagagaggatggagcttgt

SEQ ID NO: 11 - DNA, 22 bp, Artificial Sequence
  Synthetic
ggacagacaggacagtgcaaga

SEQ ID NO: 12 - DNA, 22 bp, Artificial Sequence
  Synthetic
gctcagagagggtcaggactgt

SEQ ID NO: 13 - DNA, 24 bp, Artificial Sequence
  Synthetic
agtcctcagaacaatcccatgaca

SEQ ID NO: 14 - DNA, 22 bp, Artificial Sequence
  Synthetic
ggagcctgtctgcccagtatta

SEQ ID NO: 15 - DNA, 22 bp, Artificial Sequence
  Synthetic
ctgggtggatgggagttcttgt

SEQ ID NO: 16 - DNA, 22 bp, Artificial Sequence
  Synthetic
ccactgaccacctctccaatct

SEQ ID NO: 17 - DNA, 22 bp, Artificial Sequence
  Synthetic
ctgggtggatgggagttcttgt

SEQ ID NO: 18 - DNA, 22 bp, Artificial Sequence
  Synthetic
ccactgaccacctctccaatct

SEQ ID NO: 19 - DNA, 24 bp, Artificial Sequence
  Synthetic
aagggagattatgcagtggtttgt

SEQ ID NO: 20 - DNA, 22 bp, Artificial Sequence
  Synthetic
tggaacacaggagcaggaaaca

SEQ ID NO: 21 - DNA, 23 bp, Artificial Sequence
  Synthetic
tgccaggacagagaggagtaatt

SEQ ID NO: 22 - DNA, 22 bp, Artificial Sequence
  Synthetic
aaactcccgtcccctaatgcat

SEQ ID NO: 23 - DNA, 22 bp, Artificial Sequence
  Synthetic
cacagggcatggtagacgtact

SEQ ID NO: 24 - DNA, 20 bp, Artificial Sequence
  Synthetic
cccagccctgccttcacttg

SEQ ID NO: 25 - DNA, 22 bp, Artificial Sequence
  Synthetic
cacagggcatggtagacgtact

SEQ ID NO: 26 - DNA, 20 bp, Artificial Sequence
  Synthetic
cccagccctgccttcacttg

SEQ ID NO: 27 - DNA, 19 bp, Artificial Sequence
  Synthetic
ccactcctccacctttgac

SEQ ID NO: 28 - DNA, 18 bp, Artificial Sequence
  Synthetic
accctgttgctgtagcca

SEQ ID NO: 29 - DNA, 20 bp, Artificial Sequence
  Synthetic
gggcaggttgcaaagttaga

SEQ ID NO: 30 - DNA, 20 bp, Artificial Sequence
  Synthetic
atcgttgctgaggtctgctt

SEQ ID NO: 31 - DNA, 20 bp, Artificial Sequence
  Synthetic
tcggaacaccacctggatac

SEQ ID NO: 32 - DNA, 19 bp, Artificial Sequence
  Synthetic
gatgaggagggcgagtcat

SEQ ID NO: 33 - DNA, 20 bp, Artificial Sequence
  Synthetic
cttgctgaagcacactcctg

SEQ ID NO: 34 - DNA, 21 bp, Artificial Sequence
  Synthetic
gggtgtgatctcctcattgat

SEQ ID NO: 35 - DNA, 21 bp, Artificial Sequence
  Synthetic
gctcccagaccctgaggatac

SEQ ID NO: 36 - DNA, 20 bp, Artificial Sequence
  Synthetic
cccttccccatgagtctgtc

SEQ ID NO: 37 - DNA, 21 bp, Artificial Sequence
  Synthetic
agctctcggtcaagttcaaca

SEQ ID NO: 38 - DNA, 20 bp, Artificial Sequence
  Synthetic
ttggtgaccacagcaatctt

SEQ ID NO: 39 - DNA, 18 bp, Artificial Sequence
  Synthetic
tctttcagacccggttgc

SEQ ID NO: 40 - DNA, 20 bp, Artificial Sequence
  Synthetic
ttttcaggtggtccagaagg

SEQ ID NO: 41 - DNA, 20 bp, Artificial Sequence
  Synthetic
tgatgatgagcgagatggac

SEQ ID NO: 42 - DNA, 20 bp, Artificial Sequence
  Synethic
ctgcgaagttggggtagaac

SEQ ID NO: 43 - DNA, 20 bp, Artificial Sequence
  Synthetic
gcctacgagatcaggcagtt

SEQ ID NO: 44 - DNA, 23 bp, Artificial Sequence
  Synthetic
ggtagtacttgcactgcttcagg

SEQ ID NO: 45 - DNA, 20 bp, Artificial Sequence
  Synthetic
gagaggcggctaaggtgttt

SEQ ID NO: 46 - DNA, 20 bp, Artificial Sequence
  Synthetic
gtgtagacggggatgacagg

SEQ ID NO: 47 - DNA, 20 bp, Artificial Sequence
  Synthetic
cttttgtgaaggccgaactc

SEQ ID NO: 48 - DNA, 20 bp, Artificial Sequence
  Synthetic
cacactcaggagccttttgg

SEQ ID NO: 49 - DNA, 18 bp, Artificial Sequence
  Synthetic
atgcagccctgcccagta

SEQ ID NO: 50 - DNA, 18 bp, Artificial Sequence
  Synthetic
ggcactcagcccatcaga

SEQ ID NO: 51 - DNA, 20 bp, Artificial Sequence
  Synthetic
ctggctggtctttctccttg

SEQ ID NO: 52 - DNA, 20 bp, Artificial Sequence
  Synthetic
aacctttggagaaccccagt

SEQ ID NO: 53 - DNA, 20 bp, Artificial Sequence
  Synthetic
tccgtatgttccgaggttct

SEQ ID NO: 54 - DNA, 20 bp, Artificial Sequence
  Synthetic
tttaccatgtgacgatgcaa

SEQ ID NO: 55 - DNA, 19 bp, Artificial Sequence
  Synthetic
cccagctgtgatccatgag

SEQ ID NO: 56 - DNA, 18 bp, Artificial Sequence
  Syntethic
tccaggtgaaggcgtctc

SEQ ID NO: 57 - DNA, 20 bp, Artificial Sequence
  Synthetic
atgggcagaggtgagagaga

SEQ ID NO: 58 - DNA, 20 bp, Artificial Sequence
  Synthetic
ttggctgtcagaagagagca

SEQ ID NO: 59 - DNA, 13 bp, Homo sapiens
catgaatgattca

SEQ ID NO: 60 - DNA, 9 bp, Homo sapiens
aagttctga

SEQ ID NO: 61 - DNA, 19 bp, Homo sapiens
tcttgcatctgggctcggg

SEQ ID NO: 62 - DNA, 34 bp, Homo sapiens
cgcctcccgggttcatgccattctcctgcctcag

SEQ ID NO: 63 - DNA, 57 bp, Homo sapiens
gatccaatagcaggaaaacagaagctgtcgaagaggcttcaagccatgctataatcg

SEQ ID NO: 64 - protein, 17 aa, Homo sapiens
DPIAGKQKLSKRLQAML

SEQ ID NO: 65 - DNA, 38 bp, Homo sapiens
gggtttcactatgtkggccaggcattgactaccagctc

SEQ ID NO: 66 - DNA, 30 bp, Homo sapiens
gaaggagtacattgtcagggtgatagttaa

SEQ ID NO: 67 - protein, 9 aa, Homo sapiens
EGVHCQGDS

SEQ ID NO: 68 - DNA, 52 bp, Homo sapiens
gaaggagtacattgtcaggaaaacttttctttactgactacgggaatgtcgc

SEQ ID NO: 69 - DNA, 9 bp, Homo sapiens
atgggatga

SEQ ID NO: 70 - protein, 17 aa, Homo sapiens
EGVHCQENFSLLTTGMS

SEQ ID NO: 71 - DNA, 57 bp, Homo sapiens
gaaggagtacattgtcaggataagaaactgtggtgggcagaccaaaacttagcccag

SEQ ID NO: 72 - protein, 19 aa, Homo sapiens
EGVHCQDKKLWWADQNLAQ

SEQ ID NO: 73 - DNA, 34 bp, Homo sapiens
cccaactgtgtctaagacctgggggtgggaggga

SEQ ID NO: 74 - DNA, 43 bp, Homo sapiens
ttcccaactgtgtctaagacctgggggtgggagggaagcagat

(19) United States
(12) Patent Application Publication
(10) Pub. No.: US 2017/0298437 A1
(43) Pub. Date: Oct. 19, 2017

(54) METHODS AND USES RELATED TO RHABDOID TUMORS

(71) Applicant: The Hospital For Sick Children, Toronto (CA)

(72) Inventors: Annie Huang, Toronto (CA); Jonathon Torchia, Hamilton (CA)

(21) Appl. No.: 15/239,140

(22) Filed: Aug. 17, 2016

Related U.S. Application Data
(60) Provisional application No. 62/321,935, filed on Apr. 13, 2016.

(51) Int. Cl. C12Q 1/68; A61K 31/551; A61K 31/519; A61K 31/496; A61K 38/05; A61K 31/517

(52) U.S. Cl. CPC C12Q 1/6886; A61K 38/05; A61K 31/517; A61K 31/519; A61K 31/496; A61K 31/551; C12Q 2600/106; C12Q 2600/112; C12Q 2600/154

(57) ABSTRACT

Methods and uses for diagnosing and treating rhabdoid tumors such as Atypical Teratoid Rhabdoid Tumors are provided. In particular, the present disclosure provides methods of identifying and treating subgroups of rhabdoid tumors. In one embodiment, an inhibitor that targets Notch signaling or targets an epigenetic regulator selected from a BET domain protein, G9a or EZH2, is used for treating a subject with a Group 1 type rhabdoid tumor. In another embodiment, an inhibitor that targets BMP or PDGFRβ signaling is used for treating a subject with a Group 2 type rhabdoid tumor.

### CROSS-REFERENCE TO RELATED APPLICATION

This application claims benefit under 35 U.S.C. §119(e) to U.S. Provisional Patent Application Ser. No. 62/321,935 (pending), filed Apr. 13, 2016, incorporated herein by reference in its entirety.

### INCORPORATION OF SEQUENCE LISTING

A computer readable form of the Sequence Listing “9962-P50337US01_SequenceLising.txt” (16,384 bytes), submitted via EFS-WEB and created on Aug. 12, 2016, is herein incorporated by reference.

### FIELD

The present disclosure relates to methods and uses for diagnosing and treating rhabdoid tumors, such as Atypical Teratoid Rhabdoid Tumors. In particular, the present disclosure relates to identifying and treating subgroups of rhabdoid tumors.

### BACKGROUND

Rhabdoid tumors are highly malignant neoplasms with multi-lineage histology characteristically arising in early childhood which were first described in kidneys and soft tissues, but arise most frequently in the CNS where they are called Atypical Teratoid Rhabdoid Tumors (ATRTs). ATRTs were historically considered incurable and although better outcomes has been reported with recent intensified multimodal therapy, the overall prognosis of ATRTs patients remains very poor with most succumbing within one year of diagnosis (Chi et al., 2008; Hilden, 2004; Lafay-Cousin et al., 2012; Tekautz, 2005).

Hallmark biallelic loss of function alterations of the SMARCB1 (INI1/SNF5/BAF47) tumor suppressor locus on chr22q11.23 (Versteege et al., 1998) is a diagnostic feature of ATRTs, and extra-CNS rhabdoid tumors. Up to 35% of ATRTs patients have heritable SMARCB1 alterations which predispose to multiple rhabdoid tumors (Eaton et al., 2011). Indeed Snf+/−mice also develop soft tissue or neural crest derived rhabdoid tumors (Klochendler-Yeivin et al., 2000; Roberts et al., 2002), but to date genetic models of ATRTs have not been described. SMARCB1 is a constitutive component of the SWI/SNF chromatin remodelling complex which can exhibit substantial structural and functional diversity during neurogenesis. A small proportion of ATRTs with loss of SMARCA4 (Hasselblatt et al., 2011), another component of the SWI/SNF complex, has been described and underscore the importance of epigenomic mechanisms directed by SWI/SNF in the development of ATRTs. Although collective experimental data underscore a central role for SMARCB1 in rhabdoid tumor initiation, specific mechanisms driving tumor development remains unclear. Observations that SMARCB1 deficiency leads to aberrant nucleosomal positioning of the SWI/SNF complex and is associated with upregulation of EZH2, a histone methyl transferase of the repressive PRC2 complex (Roberts and Orkin, 2004), suggest loss of SMARCB1 may lead to deregulation of multiple signaling pathways downstream of PRC2 to drive tumor development. These observations have led to therapeutic agents for rhabdoid tumors which target EZH2 and other downstream pathways (reviewed in Ginn and Gajjar, 2012).

Despite the highly malignant and heterogeneous nature of ATRTs, limited sequencing studies indicate a bland genome with only recurrent SMARCB1 mutations. Recently, it was reported that ATRTs comprised at least two transcriptional subtypes with different clinical phenotypes (Torchia et al., 2015). While group 1 ATRTs with neurogenic signatures correlated with superior survival, group 2 ATRTs with mesenchymal signatures had aggressive, treatment resistant phenotypes and dismal outcomes. However mechanisms underlying varied therapeutic responses in ATRTs patients remained unclear.

### SUMMARY

The present inventors undertook whole genome, RNAseq, global DNA methylation profiling and genome-wide high resolution chromatin openness analyses of primary ATRTs and tumor cell lines to define the genomic and epigenomic landscape of ATRT sub-groups and identify sub-group specific therapies. By integrating gene expression and methylation analyses on a large tumor cohort the present inventors demonstrate that ATRTs may be further segregated into 3 sub-groups (group 1, 2A and 2B) with distinct transcriptional and methylation signatures, global and SMARCB1 specific genotypes that correlate with age and tumor location. Global nucleosomal profiling using ATAC-seq analyses revealed distinct chromatin landscape in ATRT sub-groups, that was associated with remarkable differences in therapeutic response of sub-group specific cell lines to signaling and epigenetic inhibitors. Notably, the present inventors demonstrate using ChIP-seq and Chromatin conformational analyses that differential epigenetic regulation of a novel PDGFRβ associated enhancer confers robust sensitivity of group 2 ATRT cells to small molecule tyrosine kinase inhibitors Dasatinib and Nilotinib.

Accordingly, herein provided is use of an inhibitor that targets Notch signaling or that targets an epigenetic regulator selected from a BET domain protein, G9a or EZH2, for treating a subject with a Group 1 type rhabdoid tumor. Also provided herein is a method of treating a subject with a Group 1 type rhabdoid tumor comprising administering an inhibitor that targets Notch signaling or that targets an epigenetic regulator selected from a BET domain protein, G9a or EZH2 to the subject in need thereof. Further provided is use of an inhibitor that targets Notch signaling or that targets an epigenetic regulator selected from a BET domain protein, G9a or EZH2, in the manufacture of a medicament for treating a subject with a Group 1 type rhabdoid tumor. Even further provided is an inhibitor that targets Notch signaling or that targets an epigenetic regulator selected from a BET domain protein, G9a or EZH2, for use in treating a subject with a Group 1 type rhabdoid tumor.

In an embodiment, the inhibitor is an inhibitor of Notch signaling, such as DAPT. In an embodiment, the inhibitor is an inhibitor of a BET domain protein, such as JQ1. In an embodiment, the inhibitor is an inhibitor of G9a, such as UNC0638. In an embodiment, the inhibitor is an inhibitor of EZH2, such as UNC1999.

In one embodiment, the Group 1 rhabdoid tumor is an ATRT CNS tumor. In another embodiment, the Group 1 rhabdoid tumor is a non-CNS tumor. In a further embodiment, the Group 1 rhabdoid tumor is a soft tissue tumor such as a kidney or liver tumor.

The subject may be identified as having a Group 1 rhabdoid tumor based on genomic features, epigenomic features and/or transcriptional features. For example, the genomic features may comprise chromosome 19 loss and/or chromosome 14 gain; the epigenomic features may comprise hypomethylation of the genes listed in Table 3 or Table 5 for Group 1 subjects; and/or the transcriptional features may comprise increased level of expression of the genes listed in Table 3 or Table 5 for Group 1 subjects. Location and age may also be used to identify the tumor as a Group 1 tumor as well as other features shown in FIG. 21.

In another aspect, the present disclosure provides a use of an inhibitor that targets BMP or PDGFRβ signaling for treating a subject with a Group 2 type rhabdoid tumor, such as a Group 2A or a Group 2B rhabdoid tumor. Also provided herein is a method of treating a subject with a Group 2 type rhabdoid tumor comprising administering an inhibitor that targets BMP or PDGFRβ signaling to the subject in need thereof. Further provided is use of an inhibitor that targets a BMP or PDGFRβ signaling, in the manufacture of a medicament for treating a subject with a Group 2 type rhabdoid tumor. Even further provided is an inhibitor that targets BMP or PDGFRβ signaling, for use in treating a subject with a Group 2 type rhabdoid tumor.

In an embodiment, the inhibitor that targets BMP signaling is dorsomorphin. In an embodiment, the inhibitor that targets PDGFRβ signaling is dasatinib or nilotinib.

In one embodiment, the Group 2 rhabdoid tumor is an ATRT CNS tumor. In another embodiment, the Group 2 rhabdoid tumor is a non-CNS tumor. In a further embodiment, the Group 1 rhabdoid tumor is a soft tissue tumor such as a kidney or liver tumor.

The subject may be identified as having a Group 2 rhabdoid tumor based on genomic features, epigenomic features and/or transcriptional features. For example, the genomic features may be silent or comprise focal alterations, the epigenomic features may comprise hypomethylation of the genes listed in Table 3 or Table 5 for Group 2, Group 2A and/or Group 2B subjects and/or the transcriptional features may comprise increased level of expression of the genes listed in Table 3 or Table 5 for Group 2, Group 2A and/or Group 2B subjects. Location and age may also be used to identify the tumor as a Group 2 tumor as well as other features shown in FIG. 21.

In an embodiment, the subject is human.

Also provided herein is a method of determining the type of rhabdoid tumor of a sample comprising:

a) (i) determining a sample gene expression profile and/or (ii) a sample methylation profile from DNA from the sample, said sample gene expression profile and/or sample methylation profile comprising the level of gene expression and/or methylation, respectively, of at least three, optionally at least 5, at least 7, at least 10, at least 15, at least 20 or all of the genes listed in Table 3 or Table 5;

b) determining the level of similarity of said sample gene expression profile and/or sample methylation profile to one or more control profiles, wherein
(i) a high level of similarity of the sample profile to a Group 1 specific control profile; a low level of similarity to a Group 2A or Group 2B control profile indicates that the sample is a Group 1 rhabdoid tumor; or a higher level of similarity to a Group 1 specific control profile than to a Group 2A or Group 2B control profile indicates the sample is a Group 1 type rhabdoid tumor;
(ii) a high level of similarity of the sample profile to a Group 2A specific control profile; a low level of similarity to a Group 2B or Group 1 control profile indicates that the sample is a Group 2A rhabdoid tumor; or a higher level of similarity to a Group 2A specific control profile than to a Group 2B or Group 1 control profile indicates the sample is a Group 2A type rhabdoid tumor; or
(iii) a high level of similarity of the sample profile to a Group 2B specific control profile; a low level of similarity to a Group 2A or Group 1 control profile indicates that the sample is a Group 2B rhabdoid tumor; or a higher level of similarity to a Group 2B specific control profile than to a Group 2A or Group 1 control profile indicates the sample is a Group 2B type rhabdoid tumor.

In an embodiment, a) comprises (i) determining the sample gene expression profile and (ii) determining the sample methylation profile.

In one embodiment, determining the sample methylation profile in a)ii) comprises the steps:

a) providing the sample comprising genomic DNA from the subject;

b) optionally, isolating DNA from the sample;

c) optionally, treating DNA from the sample with bisulfite for a time and under conditions sufficient to convert non-methylated cytosines to uracils;

d) optionally, amplifying the DNA; and

e) determining the methylation status at the selected genes by means of bisulfite sequencing, pyrosequencing, methylation-sensitive single-strand conformation analysis (MS-SSCA), high resolution melting analysis (HRM), combined bisulfite restriction analysis (COBRA), methylation-sensitive single nucleotide primer extension (MS-SnuPE), base-specific cleavage/MALDI-TOF, methylation-specific PCR (MSP), methylation-sensitive restriction enzyme-based methods, microarray-based methods, whole-genome bisulfite sequencing (WGBS, MethylC-seq or BS-seq), reduced-representation bisulfite sequencing(RRBS), and/or enrichment-based methods such as MeDIP-seq, MBD-seq, or MRE-seq.

In one embodiment, the methylation level is measured as a β-value.

In an embodiment, a high level of similarity to the control profile is indicated by a correlation coefficient between the sample profile and the control profile having an absolute value between 0.5 to 1, optionally between 0.75 to 1, and a low level of similarity to the control profile is indicated by a correlation coefficient between the sample profile and the control profile having an absolute value between 0 to 0.5, optionally between 0 to 0.25. In one embodiment, the correlation coefficient is a linear correlation coefficient, optionally a Pearson correlation coefficient.

In an embodiment, the methylation levels of the selected genes of at least one control profile is derived from one or more samples, optionally from historical methylation data for a patient or pool of patients.

In an embodiment, determining the sample gene expression profile comprises measuring the expression level of the gene in the sample.

The sample may be derived from tumor tissue (for example, a biopsy) or blood.

In a further embodiment, the method comprises treating the subject with an inhibitor of Notch or an inhibitor of an epigenomic regulator if the sample is typed as a Group 1 rhabdoid tumor or treating the subject with an inhibitor of BMP or PDGFRβ signaling if the sample is typed as a Group 2A or Group 2B rhabdoid tumor.

Also provided herein are kits for practicing the methods of determining the type of rhabdoid tumor disclosed herein.

Other features and advantages of the present disclosure will become apparent from the following detailed description. It should be understood, however, that the detailed description and the specific examples while indicating embodiments of the disclosure are given by way of illustration only, since various changes and modifications within the spirit and scope of the disclosure will become apparent to those skilled in the art from this detailed description.

### BRIEF DESCRIPTION OF THE DRAWINGS

Embodiments are described below in relation to the drawings in which:

FIG. 1 shows ATRT coding genome is predominantly targeted by structural alterations. A. Box plot of global genome and CODS region somatic mutation rate in ATRTs. Median somatic mutation rates/Mb of tumor DNA were calculated using WGS and WES data available on 26 primary ATRT with matched normal lymphocyte DNA. Box boundaries represent first and third quartiles; whiskers represent minimum and maximum values. B. Circos plot summary of all recurrent structural alterations, including SCNAs and gene-rearrangements, with corresponding targeted loci as determined by integrated analyses of WGS, RNAseq, high resolution SNP and copy number derived from 450K methylation arrays performed on 180 primary ATRTs. C. SNVs and small indels associated with deleterious splice alterations, stop gain (X), and frameshift (fs) mutations in SMARCB1 are schematized in relation to DNA Binding Domain (DBD) and Repeat region 1 and 2 (Rp1, 2) domains in the SMARCB1 protein. D. Schema shows a chr22q intra-chromosomal fusion of exon 5 and 11 respectively from SMARCB1 and HORMAD2 identified by RNASeq in ATRT T51; mRNA consensus (SEQ ID NOs. 59 and 60) depicts a frameshift in HORMAD2 exon 11 which truncates the SMARCB1 protein at p.N209fs13*. E. Schematic of a chr22q intra-chromosomal translocation event involving intron 5 and 1 respectively of SMARCB1 and GTPBPI identified by WES in primary ATRT (T12). Translocation fusion product predicted by CREST to disrupt SMARCBI coding sequence is shown relative to a consensus sequence (SEQ ID NO. 61) of respective gene fragments with a common CTG tri-nucleotide.

FIG. 2 shows ATRTs comprise 3 epigenetic sub-groups with distinct clinical profiles and genotypes. A-B. Molecular sub-groups of ATRTs were identified by unsupervised consensus hierarchical (HCL) (top) and Non-negative Matrix Factorization (NMF) (bottom panel) consensus cluster analyses of global methylation (A; Illumina 450 k; n=162 tumors) or gene expression (B; Illumina HT12; n=90 tumors) data. Consensus HCL was used to determine k=3 as the optimal number of classes. Tumor grouping based on gene expression or methylation status of respectively 250 genes and 10,000 methylation probes are indicated for group 1, group 2A, or group 2B boxes. An adjusted Rand index of 0.6129; indicated tight concordance of gene expression and methylation clusters. NMF consensus cluster analyses revealed highest co-phenetic coefficients (k=3) for gene expression and methylation with 250 genes and 10,000 probes, respectively. C. Summary of clinical, molecular and recurrent genotypic features in 177 primary ATRTs with subgroups determined by methylation or gene expression. Molecular grouping of tumors are indicated for group 1, group 2A, group 2B. Clinical (tumor location, patient age, metastatic status) and global patterns of copy number alterations (chromosomal or sub-chromosomal/focal) and type of SMARCB1 alterations in individual tumors are indicated. Clinical or molecular features significantly correlated with ATRT sub-groups are indicated. SMARCB1 alterations determined by at least 2 molecular techniques were classified as focal (point mutations, small indels, intergenic deletions) or broad (intragenic events, large deletions). D. Left panel: Pie chart shows proportion of supra-tentorial, infra-tentorial and spinal tumors within ATRT sub-groups. Middle and Right panel show plots of median patient age and age distribution in tumor sub-groups. Fisher's exact and Kruskal-Wallis test were used respectively to calculate significance of tumor location and age in ATRT sub-groups; p value <0.5 was considered significant.

FIG. 3 shows ATRT sub-groups have lineage enriched transcriptional and methylation signatures. A. Star burst plot of ATRT sub-group specific genes with reciprocal changes in DNA methylation (x-axis) and gene expression (y-axis). Genes differentially associated with group 1 (left panel), group 2A (middle panel), and group 2B (right panel) ATRTs, which include neural/mesenchymal cell lineage (ASCL1/OTX2/HOX) and (NOTCH/BMP/PDGFRβ) signaling pathways, are highlighted. B. Ingenuity Pathway Analysis (IPA) of gene expression data was used to identify enriched and upregulated pathways in ATRT subgroups. Student's t test with FDR correction identified genes most significantly upregulated with respect to other subgroups. Genes with fold change ±2 fold were used as input for IPA. The top 10 pathways identified by βlog p value (top axis) are shown for each subgroup; ratio (enrichment) of genes within each pathway is depicted on bottom axis. C. Gene expression heat map of sub-group enriched neural/mesenchymal lineage and NOTCH/BMP/HOX signaling genes in ATRT; gene enrichments were determined using supervised t test with FDR correction. Genes upregulated across subgroups 2A and 2B are bolded and indicated by a dashed box. D. Heatmaps show methylation levels of representative lineage genes in ATRT sub-groups, methylation status of probes in ASCL1, OTX2 and HOXB2, are shown relative to transcriptional start sites.

FIG. 4 shows molecular sub-groups of ATRTs have different chromatin landscape and functional genomes. A. Principle component (PCA) and correlation analysis of ATAC-seq data from 5 primary ATRTs and 4 ATRT cell lines. Aligned sequence reads from ATAC-seq profiling were converted to peak tag counts using HOMER software for PCA and correlation analysis using DiffBind software; sample relatedness are indicated. B. Global chromatin openness profiles determined using ATAC-seq analyses in primary group 1 (ATRT 78, 35), 2A (ATRT 40, 41) and 2B (ATRT 35) ATRTs. Differentially open-chromatin peaks (FDR<0.5) were identified using Peak analysis and DiffBind. Peaks were annotated with corresponding gene relative to the peak center, and heatmap was generated by averaging the peak read density in 20 bp bins in a range ±2.5 kb from the peak center. RNAseq profiles for corresponding genes in each tumor are plotted as an FPKM heat map alongside ATAC-seq data. Scale is proportional to read enrichment and normalized between ChIP-seq experiments for relative to input DNA. C-D. Individual ATAC-seq alignment tracks for subgroup-specific lineage (C) and signaling (D) genes in primary tumors and cell lines. Alignment Bam files were converted to bedgraph format for visualization in the Integrated Genomics Viewer (IGV) software. Tracks are shown relative to RefSeq gene annotations for genome version hg19.

FIG. 5 shows NOTCH and BMP inhibitors abrogate ATRT growth and migratory phenotypes in a sub-group specific manner. A. Molecular sub-typing of 10 ATRT cell lines determined using gene expression analyses and confirmed by Western blot analyses for NOTCH intracellular domain (NICD) and phospho-SMAD1/5. UW228medulloblastoma cell line lysates was used as positive control for SMARCB1 immuno-staining and tubulin served as loading control. B. MTS assays of group 1 and 2 ATRT cell lines respectively at 3 and 5 days post treatment with DAPT and Dorsomorphin (DM), cell viability is normalized to corresponding DMSO treated controls. Error bars represent SEM from 3 experiments with 2 replicas/data point. Significance was calculated using Student's t test; *=p<0.05. C-D. Effect of DAPT and DM on NOTCH and BMP signaling in ATRT cell lines was confirmed by qRT-PCR analyses of respective target genes and Western blot analyses for NICD and phospho-SMAD1/5 in treated group 1 and 2 ATRT cell lines. Cell lines were treated with increasing doses (black triangle) of DAPT or DM, and cross treated with a single dose of DM or DAFT; ±signs indicate presence or absence of specific drugs. mRNA levels are normalized relative to actin, and to carrier treated control cells (black bar). Error bars=SEM for 3 experiments with 3 replicas/data point. Significance was calculated using Student's t test. E. TUNEL assay shows DAPT and DM induced apopotosis respectively in group 1 (CHLA02) and 2 (SH) ATRT cell lines. Western blot of NICD and pSMAD1/5 confirmed DAPT and DM induced apopotosis respectively via NOTCH and BMP signaling. F. Left panel: Bar graph summary of transwell cell migration assays of group 1 and 2 ATRTs with corresponding images of migrated cells. Right panel: Bar graph summary of transwell cell migration assay and Western blot analyses of pSMAD1/5 performed after DM treatment. Error bars=SEM for 3 experiments with 2 replicas per data point; significance was calculated using a Student's t test.

FIG. 6 shows ATRTs exhibit sub-group specific therapeutic response to signaling and epigenetic pathway inhibitors. MTS assays were performed on 8 sub-type specific ATRT cell lines after treatment with varying doses of 18 small molecule inhibitors/chemical probes targeting PDGF/Src tyrosine kinases (Dasatinib and Nilotinib), G9a (UNC0638), BET domain protein (JQ1), EZH2 (UNC1999) and HDAC (LAQ824) (see FIG. 18 for full figure). Viability of treated cells was determined relative to DMSO treated controls over 5-7 days. Error bars=SEM for 3 experiments with 2 replicas/data point; p<0.05 calculated using Student's t test was considered significant for treatment effects. A. Sub-group specific therapeutic effects on ATRT cell lines observed with select probes/drugs is summarized. + or − indicates significant or non-significant effects on cell viability based on MTS assays, + indicates >30% reduction in cell viability with varying doses of indicated drug/probe. B. MTS assays shows viability of group 1 and 2 ATRT cell lines 7 days post-treatment with UNC0638, JQ1 and UNC1999. C. MTS assays showing viability of effect group 1 and 2 ATRT cell lines 5 days post-treatment with Dasatinib and Nilotinib. D. Gene expression heat map showing highest relative expression of candidate receptor tyrosine and cytosolic kinases targeted by Dasatinib/Nilotinib in primary group 1 and 2 ATRTs. Significance was determined using Student's t test adjusted for FDR. PDGFRβ shown was most significantly upregulated in group 2 ATRTs.

FIG. 7 shows epigenetic regulation of a novel enhancer element underlies sensitivity of group 2 ATRTs to small molecule inhibitors of PDGFRβ signaling. A. UCSC genome browser map of the CSF1R/PDGFRβ locus is shown relative to tracks from UCSC and/or ENCODE databases. Schema of CSF1R/PDGFRβ with flanking genes (chr5:149,370,252-149,566,612) is shown with a zoomed view of the putative regulatory domain/enhancer element relative to exon 1 and gene body of CSF1R and the PDGFRβ promoter (chr5:149,479,360-149,545,365). Illumina 450 k probe locations, DNAsel hypersensitivity, and ENCODE cell line tracks for H3K27ac, H3K4Me1 and H3K4 me3 ChIP-seq are shown below. Arrows indicate putative chromatin looping indicated by 3C analyses. (see C). B. IGV alignment tracks of primary and cell line ATAC-seq data for the CSF1R/PDGFRβ is depicted. Bottom track represents H3K27Ac ChIP-seq peak performed on BT12, a Dasatinib-sensitive group 2 ATRT cell line. Box depicts the putative regulatory domain/enhancer element with corresponding schematic of probes hypomethylated in group 2 ATRTs. C. Scatter plots of log₂ gene expression (y-axis) and beta value methylation score (x-axis) for 75 ATRTs with gene expression and methylation data are shown for probes within the enhancer domain, PDGFRβ gene body, North (N) shore, CpG island and PDGFRβ promoter regions. Pearson's correlation coefficient (R²) and linear regression line indicates correlation of gene expression and methylation levels. Approximated location of differentially methylated probes within the CSF1R-PDGFRβ locus is schematized.

FIG. 8 shows chromatin structure and protein expression of PFDGRβ. A. 3C Chromatin Conformation Capture analyses of ATRT cell lines CHLA05 and BT12. Lines denote location of PCR primers mapping to enhancer domain and the PDGFRβ promoter. Plot indicates relative co-amplification and predicted interaction of anchor:test primer pairs designed at various distances relative to the anchor primer as indicated on the x-axis. Grey bars highlight location of test primers relative to CSF1R/PDGFRβ. B. Schematic representation of 3C analysis results which predicts DNA looping and direct interaction of the PDGFRβ promoter and an active enhancer mapping 50 kbs upstream. C. Western blot analyses of phospho-PDGFRβ in group 1 and 2 ATRT cell lines. D. Western blot analyses of phospho-PFDGRβ in group 2 ATRT cell lines 3, 5 and 7 days post-treatment with 50 nM of Dasatinib and DMSO control; tubulin served as protein loading control.

FIG. 9 shows landscape of somatic mutations in ATRTs. A. Mutations with a somatic score ≧35 were selected for visual validation of somatic status in IGV, and the sensitivity/specificity for the CLR score in accurately identifying somatic mutations was calculated and plotted (see methods). A CLR score of 68 had the highest sensitivity without significant loss of specificity (89% for both sensitivity and specificity). B. Top panel shows frequency of indels and SNV across the genome (left y-axis) and CODS (right y-axis) regions for individual tumors. Single nucleotide transitions and transversion profiles calculated/genome for corresponding tumors is shown in the bottom panel. Relative nucleotide transitions and transversion rates in the genome (hatched) and CODS coding region (solid) are shown in the bottom right panel. C. Relative nucleotide transitions and transversion rates in the recurrent (hatched) and primary tumors (solid). D. Numbers of somatic SNVs and indels in whole exome sequencing data.

FIG. 10 shows novel genetic alterations of SMARCB1. A. Tandem duplication of exon 6 in SMARCB1 identified by WGS analysis of ATRT T22 was indicated by increased Log2 ratio (tumor:matched lymphocyte) of sequence depth. Eleven discordant read pairs identifying the centromeric and telomeric boundaries of tandem duplication event are indicated. Bottom plot shows alignment of paired-end reads with discordant read pairs highlighted. B. Tandem duplication encompassing exons 4 and 5 of SMARCB1 (SEQ ID NO. 62) identified by WGS analyses in ATRT T36. C. RNA sequencing read depth of transcribed SMARCB1 (grey; exon 1-5 average depth of 70.82) and HORMAD2 (exon 11 average depth of 150) exons are shown relative to transcript maps. D. PCR-Sanger sequence confirmation of a novel tumor specific 110 bp DNA fragment from the SMARCB1:HORMAD2 intra-chromosomal translocation in ATRT T51; chromatogram shows representative flanking SMARCB1 and HORMAD2 sequences (SEQ ID NOs: 59 and 60). E. IGV alignment read depth for the SMARCB1 gene showing gain of exons 1-5 in ATRT T12. Sequence in chromatogram is SEQ ID NO. 61. F. PCR-Sanger sequence confirmation of a tumor specific 251 bp SMARCB1:GTPBP1 breakpoint fusion product (arrow); a common CTG tri-nucleotide at the translocation breakpoint is indicated in a chromatogram; +/−indicates DNA strand orientation.

FIG. 11 shows copy number-driven changes in gene expression on chr22. A. Composite copy number heat map of recurrent chr 22 SCNAs in a subset of 25 ATRTs with RNAseq data, which encompasses several coding target loci in addition to SMARCB1. Copy number status was determined by multiple methods including OmniQuad SNP copy number array, Illumina methylation 450 k array, and whole genome sequencing (see methods). Loci with minimal mRNA expression levels across all ATRTs, as determined using gene expression arrays/RNAseq and/or RT-PCR analyses, are indicated in grey. Relative positions of RefSeq loci are indicated. B. Box plots showing FPKM values for each gene in the chr22q11.23 and q13.1-2 intervals, grouped by copy number status. C. Heat map of contiguous and non-contiguous deletions of BCR and SMARCB1 in ATRT T52, T32 and T5, relative to ATRT T25 which harbours a SMARCB1 point mutation and no chromosome 22 copy number changes. D. qRT-PCR analyses which confirm a focal non-contiguous deletion of BCR and SMARCB1 in ATRT T52. Expression of specific BCR and SMARCB1 exons and intervening loci, RGL4, CHCHD10 and MMP11 were determined relative to a normal fetal brain control using the ΔΔCT method, error bars represent standard deviation. E. Heat map indicating overlapping focal heterozygous deletions of MKL1 and EP300 in ATRT T2 and T32 relative to a tumor T25 which is diploid for both genes. Corresponding RefSeq gene tracks and genomic positions are indicated. F. Corresponding RNAseq read depths plots for MKL1 and EP300 exons in ATRT T2, T32, T25 and control fetal brain relative to RefSeq gene tracks.

FIG. 12 shows focal genomic alterations of tumor suppressor LRP1B and CDH13 in ATRTs. A. Copy number heat map of locus chr 2q21.3-q23.1 in a subset of primary ATRTs, and a magnified view for five ATRT samples with intragenic heterozygous deletions of LRP1 B. Copy number analyses of ATRT T22 and matched normal lymphocyte DNA, indicate LRP1B deletion as specific somatic event. B. Log2 ratio plots of WGS data from ATRT T22 and matched normal lymphocyte DNA indicating decreased sequence coverage due to a 612 kb tumor specific intragenic deletion encompassing exons 8-46 of LRP1 B (SEQ ID NOs: 63 and 64). Bottom panel shows PCR-Sanger sequencing and corresponding chromatogram which confirmed a 400 bp tumor specific fragment (arrow) generated by the focal deletion that results in a frameshift (p.G339fs12*) and stop gain (*) sequence alteration in the LRP1B protein (SEQ ID NO. 65). C. Schema of LRP1B deletions in ATRT T21 (SEQ ID NOs. 66 and 67) and T45 (SEQ ID NOs. 68, 69 and 70), and deletions of LRP1B ex4-32 seen in two tumors, T14 and T17. (SEQ ID NOs. 71 and 72). Lines indicate approximate intragenic deletion boundaries as determined by SNP array analyses. LRP1 B exon deletions and fusions are predicted to result in premature stop codons in tumors T21 and T45, and in-frame 221 amino acid deletions in tumors T14 and T17. D. Copy number heat map of 2 ATRTs generated using array segmentation analyses indicate a minimal region of deletion on chr16q23 mapping to CDH13. E. Inter-chromosomal translocation of chr 11p15.4 intergenic sequence with CDH13 coding sequence on chr16q23.3 in a recurrent ATRT T42R. Schema shows fusion event and corresponding IGV view of 40 discordant read-pairs spanning the translocation breakpoint (average coverage of 58.7X) together with a consensus sequence assembled using CREST (SEQ ID NOs. 73 and 74). Sanger sequencing confirmed a novel 391 bp (arrow) fusion product; DNA strands and gene orientation are respectively indicated by +/− and arrows.

FIG. 13 shows defining molecular classes of ATRT by global methylation and gene expression analyses. Multiple unsupervised consensus hierarchical cluster (HCL) analyses were performed on global methylation data generated from 162 primary ATRTs using the Illumina 450K BeadChip methylation arrays and 90 primary ATRTs using the Illumina HT12 Gene expression microarray. In order to discover molecular classes of ATRTs, an initial set of 30,000-6,000 probes and 2,000-250 genes with the highest standard deviation was re-iteratively analysed to determine the most stable tumor group clusters achievable with a minimal probe set. A. CDF plots of multiple unsupervised HCL cluster analyses performed on methylation data using 6,000-14,000 probes and 250-2,000 genes ranked by standard deviation to establish the most robust number of k classes, which was determined to be k=2 or 3 classes. B. Consensus HCL matrices for the individual probe sets for k=2 and k=3 classes. Black indicates samples which remain stable within each cluster over 1000 iterations after 80% resampling. Lighter black indicates samples which distribute between multiple groups. K=3 was selected as the optimal number of k-classes. C. NMF consensus clustering was performed for k=3 for each probe set to establish the most robust subgrouping. Consensus matrices for each probe set determined that 10,000 probes and 250 genes gave the highest co-phenetic coefficient. D. Cluster assignments for consensus NMF and HCL are shown with a rand index of 0.8163 (methylation) and 0.7453 (gene expression) indicating a high agreement between orthogonal methods. E. Silhouette analysis for 10,000 probes and 250 genes indicated five and two samples with a silhouette width below 0 for methylation and gene expression respectively, and were removed from further statistical analyses. F. Methylation profiles of 162 primary CNS-ATRTs and 9 extra-CNS rhabdoid tumors were compared by unsupervised hierarchical cluster analyses of the top 10,000 most variable probes ranked by standard deviation.

FIG. 14 shows global copy number and methylation features of ATRT sub-groups. A. CGH plot of copy number events for each ATRT subgroup is shown. Y-axis depicts the frequency of events over each chromosome on the X-axis. Chromosome 14 gain and chromosome 19 loss were specific for group 1 ATRTs (p=0.0142 and p=0.0325 for chromosome 14 and 19, respectively). B. Overall (left panel) and CpG island (right panel) methylation levels are shown for ATRT subgroups relative to other pediatric brain tumors (PNET; primitive neuro-ectodermal tumor, MB; medulloblastoma, GBM; glioblastoma, and EPEND; ependymoma). Methylation levels are represented as β-values and are normalized using BMIQ method (see methods). ATRTs are significantly hypermethylated overall relative to the other 4 brain tumors. Group 2A has significantly less hypomethylation of CpG islands relative to the two other ATRT subgroups.

FIGS. 15 and 16 shows methylation levels at promoters of representative loci in ATRT subgroups. Heatmap of probe methylation levels of promoter regions are shown for representative loci group 1 (ASCL1, FABP7, DLL1), group 2 (SERPINF1, BMP4), group 2A (OTX2, CLIC6, CLDN3), and group 2B (HOXB2, HOXC4/5/6) with corresponding scatter plot of log2 gene expression and beta value methylation score (right panel). Average methylation levels of probes at the promoter +/−2.5 kb from TSS (encompassing north/south shore and CpG islands) are plotted against gene expression levels for each individual tumor; a corresponding regression line and Pearson's correlation value (R2) are indicated. Schema shows locations of TSS and methylation probes in individual loci.

FIG. 17 shows treatment of Group 1 and 2 cell lines with DAPT and DM. A. Supervised cluster analyses using the top 250 genes identified from primary ATRT HCL analyses. B-C. MTS cell viability assay of group 1 (Panel B; CHLA02, CHLA05, 78C) or group 2 (Panel 0; SH, CHLA06, BT12, BT16) ATRT cell lines treated with gamma-secretase inhibitor DAPT and BMP pathway inhibitor Dorsomorphin (DM) for 5 days. Error bars represent SEM with 3 biological replicates and 3 replicas/time point; differences in cell viability were assessed using the Student's independent t test with a significance threshold of p<0.05.

FIG. 18 shows summary of screening for ATRT subgroup-specific therapeutic targets. A. MTS cell viability assays were conducted for various small molecules for group 1 (CHLA02, CHLA04, CHLA05, CHLA266) and group 2 (CHLA06, BT16, SH, BT12) ATRT cell lines. For each drug and each cell line, 4 concentrations (0.1, 0.25, 0.5, 1.0 pM [SGC946, JQ1, UNC642, PFI1, A366, UNC638], 0.5, 1.0, 2.5, 5.0 pM [GSK2801, GSK343, SGCCBP30F, UNC1999, LAQ824, PFI2], 0.1, 0.5, 1.0, 2.0 μM [LLY507], 0.5, 2.5, 5.0, 10.0 μM [J4]) of inhibitor were tested and MTS absorbance readings were taken at day 3 and day 7 post treatment. Greater than 30% reduced cell viability is indicated with a plus (+) sign. Highlighted in bold are drugs active across both ATRT subgroups. na; failed due to lack of growth, *; universally toxic at low concentrations. B. Bar plot summary of MTS assay for a subset of probes identified with robust and consistent subgroup-specific effects on ATRT cell viability. Dashed line indicates threshold of 30% reduced cell viability, with p values indicating significantly decreased (p<0.05) cell viability relative to DMSO-treated negative controls.

FIG. 19 shows treatment of Group 1 and 2 cell lines with Dasatinib and Nilotinib. MTS cell viability assay of group 1 (78C, CHLA02, CHLA05) or group 2 (SH, CHLA06, BT12, BT16) ATRT cell lines treated with Dasatinib and Nilotinib for 5 days. Error bars represent SEM with 3 biological replicates and 3 replicas/time point; differences in cell viability were assessed using the Student's independent t test with a significance threshold of p<0.05.

FIG. 20 shows a schema of the PDGFRβ locus. A. UCSC gene tracks are shown for CSF1R-PDGFRβ locus (chr5:149,479,360-149,545,365). Tracks shown (top to bottom) include RefSeq gene annotation, Illumina 450 k probe addresses, and regulation tracks from the ENCODE project including DNase hypersensitivity sites, H3K27ac, H3K27me1, H3K27me3, and transcription factor ChlPseq data. Lines depict the predicted enhancer domain, PDGFRβ gene body, and PDGFRβ promoter regions. Arrows indicate predicted chromatin interactions which form a putative gene loop (see FIG. 8B). B. Gene expression heatmap of transcription factors predicted to bind to the PDGFRβ promoter/enhancer domain in 90 primary ATRT samples. MYC and FOS are upregulated in group 2 ATRTs and are putatively involved in PDGFRβ transcription. Fold change (Group 1 vs. Group 2) and FDR corrected p values were calculated using a supervised Student's t test; p<0.05 was considered significant. C. Box plots depict normalized expression values for PDGFRβ, CSF1R and D. TYR in ATRT subgroups. Hatches represent minimum and maximum expression values, upper and lower box limits respectively represent the 25th and 75th quartiles. Middle bar represents the average value Student's t test with FDR correction was used to test for differences in average expression between ATRT subgroups, with p<0.05 considered significant.

FIG. 21 is a graphical abstract setting out the features that have been found in Group 1, Group 2A and Group 2B rhabdoid tumors.

FIG. 22 shows assessment of prediction accuracy for various gene sets.

### DETAILED DESCRIPTION

The present inventors have determined subgroups of rhabdoid tumors based on a number of features including gene expression and have further determined subgroup-specific therapeutic treatment options.

Accordingly, herein provided is use of an inhibitor that targets Notch signaling or that targets an epigenetic regulator selected from a BET domain protein, G9a or EZH2, for treating a subject with a Group 1 type rhabdoid tumor. Also provided herein is a method of treating a subject with a Group 1 type rhabdoid tumor comprising administering an inhibitor that targets Notch signaling or that targets an epigenetic regulator selected from a BET domain protein, G9a or EZH2 to the subject in need thereof. Further provided is use of an inhibitor that targets Notch signaling or that targets an epigenetic regulator selected from a BET domain protein, G9a or EZH2, in the manufacture of a medicament for treating a subject with a Group 1 type rhabdoid tumor. Even further provided is an inhibitor that targets Notch signaling or that targets an epigenetic regulator selected from a BET domain protein, G9a or EZH2, for use in treating a subject with a Group 1 type rhabdoid tumor.

The term “rhabdoid tumor” as used herein refers to a highly malignant neoplasm that characteristically arises in early childhood. Rhabdoid tumors are often identified as aggressive soft tissue sarcomas in children with alternations in SMARCB1 (also known as INI1/SNF5/BAF47). A rhabdoid tumor may be identified, for example, by assessing the status of SMARCB1 in a tumor biopsy sample. Types of rhabdoid tumors include central nervous system Atypical Teratoid Rhabdoid tumor (ATRTs) as well as non-CNS rhabdoid tumors such as kidney, liver and other soft tissues.

Accordingly, in one embodiment, the Group 1 rhabdoid tumor is an ATRT CNS tumor. In another embodiment, the Group 1 rhabdoid tumor is a non-CNS tumor, such as a renal rhabdoid tumor or other soft tissue rhabdoid tumor for which there is no internationally established subgrouping.

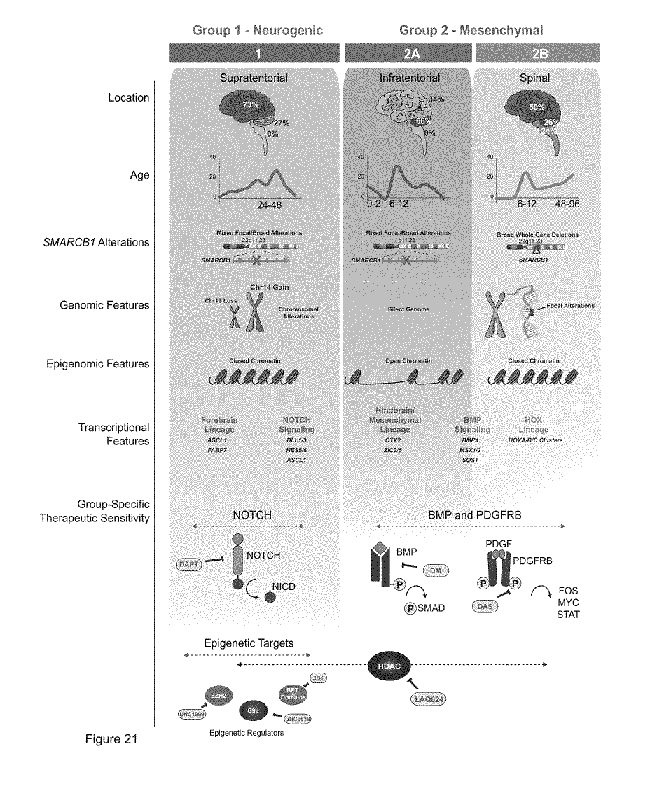

The present inventors have shown that rhabdoid tumors may be grouped, not only based on location, age, and SMARCB1 alterations, but also based on certain genomic, epigenomic and transcriptional features, for example as laid out in FIG. 21.

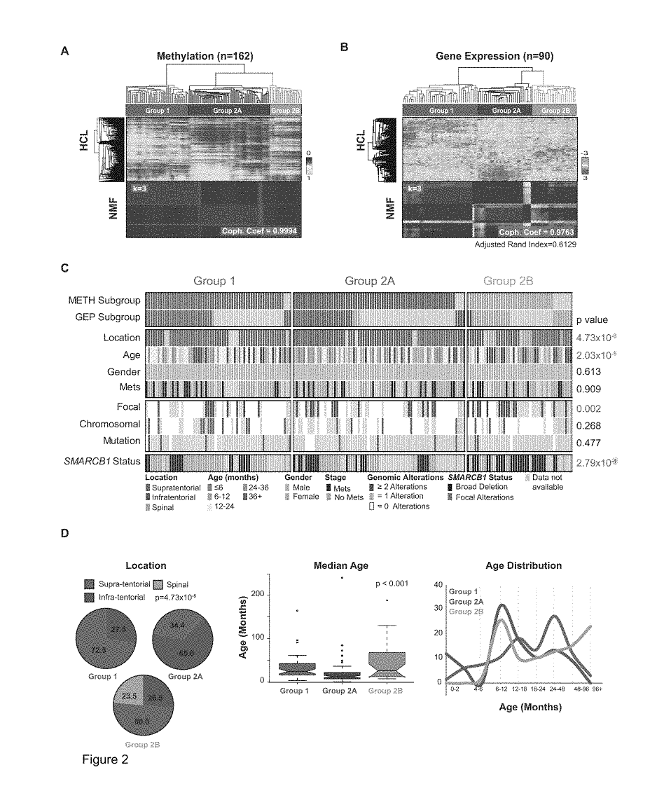

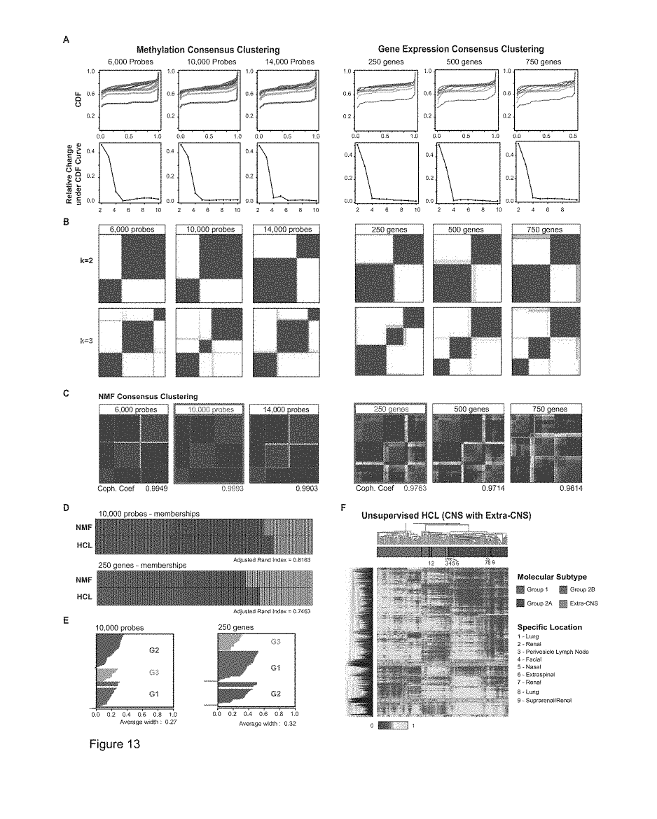

A subject may be identified as having a Group 1 rhabdoid tumor by using the 250 most variable (coefficient of variation) genes from a gene expression microarray (for example, the Illumina human HT12 array; see FIG. 2A and FIG. 13) or the most variable 10,000 probes (standard deviation) from a methylation microarray (for example, the Illumina 450K BeadChip methylation array, see FIG. 2B and FIG. 13) in unsupervised consensus hierarchical clustering analyses (HCL). HCL analyses will segregate ATRTs into three subgroups which can be then be used to determine differential expression/methylation between the three subgroups. A supervised t test (adjusted for multiple hypotheses with the false discovery rate [FDR] method) is used to identify the most upregulated genes between subgroups, whereas a Wilcox Rank Sum test is used to identify the most differentially methylated probes. In each case, one group is compared to the other two subgroups. The identity of a Group 1 ATRT is based on upregulation/hypomethylation of genes implicated in neural development and NOTCH signaling and can include, for example, the genes listed in Table 3 or Table 5 as for Group 1 subjects. In addition, the features shown in FIG. 21, including genomic features, epigenomic features and/or transcriptional features are characteristic of Group 1 rhabdoid tumors. For example, the genomic features may comprise chromosome 19 loss and/or chromosome 14 gain; the epigenomic features may comprise hypomethylation of the genes listed in Table 3 or Table 5 for Group 1 subjects; and/or the transcriptional features may comprise increased level of expression of the genes listed in Table 3 or Table 5 for Group 1 subjects. Increased expression and hypomethylation is relative to the other groups. For example, increased expression or hypomethylation in Group 1 is relative to Group 2, including Group 2A and Group 2B. In an embodiment, the subject is identified as Group 1 using the methods disclosed herein for determining type of rhabdoid tumor.

The term “inhibitor” as used herein refers to an agent that reduces, decreases, or otherwise blocks expression or activity of its target, and includes any substance that is capable of inhibiting the expression or activity of the target and includes, without limitation, antisense nucleic acid molecules, siRNAs or shRNAs, proteins, antibodies (and fragments thereof), small molecule inhibitors and other substances directed at the target expression or activity.

In an embodiment, the inhibitor is an inhibitor of Notch signaling. The Notch signaling pathway is a highly conserved cell signaling system present in most multicellular organisms. Notch signaling inhibitors are known in the art. In one embodiment, the Notch signaling inhibitor is DAPT or a γ secretase inhibitor such as Dibenzazepine. In another embodiment, the Notch signaling inhibitor is an antisense nucleic acid molecule of a gene encoding a protein in the Notch signaling pathway or is a shRNA molecule that inhibits the expression of a protein in the Notch signaling pathway. Examples of genes encoding proteins in the Notch signaling pathway include NOTCH1 (AF308602.1) and NOTCH2 (AF315356.1). In yet another embodiment, the inhibitor is an antibody specific to a protein in the Notch signaling pathway or one of its ligands.

In an embodiment, the inhibitor is an inhibitor of a BET domain protein, such as JQ1 ((S)-tent-butyl 2-(4-(4-chlorophenyl)-2,3,9-trimethyl-6H-thieno[3,2-f][1,2,4]triazolo[4,3-a][1,4]diazepin-6-yl)acetate). In another embodiment, the inhibitor is an antisense nucleic acid molecule of the gene encoding the BET domain protein or a siRNA molecule that inhibits expression of the BET domain protein. In yet another embodiment, the inhibitor is an antibody specific to the BET domain protein. Examples of BET domain proteins include, but are not limited to, BRD2 (BC063840.1), BRD3 (BC032124.2), BRD4 (AF386649.1) and BRDT (AF019085.1).

In an embodiment, the inhibitor is an inhibitor of G9a (EHMET2; EU070918), such as UNC0638. In another embodiment, the inhibitor is an antisense nucleic acid molecule of the gene encoding G9a or a siRNA molecule that inhibits expression of G9a. In yet another embodiment, the inhibitor is an antibody specific to G9a.

In an embodiment, the inhibitor is an inhibitor of EZH2 (EZH2; uc003wfb.2), such as UNC1999. In another embodiment, the inhibitor is an antisense nucleic acid molecule of the gene encoding EZH2 or a siRNA molecule that inhibits expression of EZH2. In yet another embodiment, the inhibitor is an antibody specific to EZH2.

In another aspect, the present disclosure provides a use of an inhibitor that targets BMP or PDGFRβ signaling for treating a subject with a Group 2 type rhabdoid tumor, such as a Group 2A or a Group 2B rhabdoid tumor. Also provided herein is a method of treating a subject with a Group 2 type rhabdoid tumor comprising administering an inhibitor that that targets BMP or PDGFRβ signaling to the subject in need thereof. Further provided is use of an inhibitor that targets BMP or PDGFRβ signaling, in the manufacture of a medicament for treating a subject with a Group 2 type rhabdoid tumor. Even further provided is an inhibitor that targets BMP or PDGFRβ signaling, for use in treating a subject with a Group 2 type rhabdoid tumor.

In an embodiment, the inhibitor that targets BMP signaling is dorsomorphin. In another embodiment, the inhibitor is an antisense nucleic acid molecule of the gene encoding a bone morphogenetic protein (BMP) or a bone morphogenetic protein receptor (BMPR) or a siRNA molecule that inhibits expression of a BMP or its receptor. In yet another embodiment, the inhibitor is an antibody specific to a BMP or its receptor including but not limited to BMP1 (BC143338.1), BMP2 (KC294426.1), BMP3 (BC117514.1), BMP4 (EU518936.1), BMP5 (KC485577.1), BMP6 (NM_001718.4), BMP7 (BC008584.1), BMP9 (AF188285.1), BMPR1A (BCO28383.1), BMPR1B (BC069796.1) and BMPR2 (NM_001204.6).

In an embodiment, the inhibitor that targets PDGFRβ (PDGFRβ; UC003Iro.3) is dasatinib or nilotinib. In another embodiment, the inhibitor is an antisense nucleic acid molecule of the gene encoding PDGFRβ or its ligand or a shRNA molecule that inhibits expression of PDGFRβ or its ligand. In yet another embodiment, the inhibitor is an antibody specific to PDGFRβ or its ligand.

In one embodiment, the Group 2 rhabdoid tumor is an ATRT CNS tumor. In another embodiment, the Group 2 rhabdoid tumor is a non-CNS tumor.

In one embodiment, a subject may be identified as having a Group 2 rhabdoid tumor by using the 250 most variable (coefficient of variation) genes from a gene expression microarray (for example, the Illumina human HT12 array; see FIG. 2A and FIG. 13) or the most variable 10,000 probes (standard deviation) from a methylation microarray (for example, the Illumina 450K BeadChip methylation array, see FIG. 2B and FIG. 13) in unsupervised consensus hierarchical clustering analyses (HCL). HCL analyses will segregate ATRTs into three subgroups which can be then be used to determine differential expression/methylation between the three subgroups. A supervised t test (adjusted for multiple hypotheses with the false discovery rate [FDR] method) is used to identify the most upregulated genes between subgroups, whereas a Wilcox Rank Sum test is used to identify the most differentially methylated probes. In each case, one group is compared to the other two subgroups. The identity of a Group 2 ATRT is based on upregulation/hypomethylation of genes implicated in BMP signaling and mesenchymal differentiation, and can include, for example, the genes listed in Table 3 or Table 5 as for Group 2 subjects. In addition, the features shown in FIG. 21, including genomic features, epigenomic features and/or transcriptional features are characteristic of Group 2 rhabdoid tumors, including genomic features, epigenomic features and/or transcriptional features. For example, the genomic features may be silent or comprise focal alterations, the epigenomic features may comprise hypomethylation of the genes listed in Table 3 or Table 5 for Group 2, Group 2A and/or Group 2B subjects and/or the transcriptional features may comprise increased level of expression of the genes listed in Table 3 or Table 5 for Group 2, Group 2A and/or Group 2B subjects. For example, increased expression or hypomethylation in Group 2A is relative to Group 2B and Group 1; increased expression or hypomethylation in Group 2B is relative to Group 2A and Group 1 and increased expression or hypomethylation in Group 2 is relative to Group 1. In an embodiment, the subject is identified as Group 2, 2A or 2B using the methods disclosed herein for determining type of rhabdoid tumor.

The term “treating” as used herein refers to improving the condition, such as reducing or alleviating symptoms associated with the condition or improving the prognosis or survival of the subject.

Conventional treatment may also be used in combination with the methods and uses of the disclosure. The currently used agents used for treatment of rhabdoid tumors, include without limitation, cisplatin, etoposide, vincristine, ifosfamide, doxorubicin, actinomycin, cyclophosphamide, and intrathecal agents.

The term “nucleic acid molecule” is intended to include unmodified DNA or RNA or modified DNA or RNA. For example, the nucleic acid molecules or polynucleotides of the disclosure can be composed of single- and double stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically double-stranded or a mixture of single- and double-stranded regions. In addition, the nucleic acid molecules can be composed of triple-stranded regions comprising RNA or DNA or both RNA and DNA. The nucleic acid molecules of the disclosure may also contain one or more modified bases or DNA or RNA backbones modified for stability or for other reasons. “Modified” bases include, for example, tritiated bases and unusual bases such as inosine. A variety of modifications can be made to DNA and RNA; thus “nucleic acid molecule” embraces chemically, enzymatically, or metabolically modified forms. The term “polynucleotide” shall have a corresponding meaning.

The term “antisense nucleic acid” as used herein means a nucleotide sequence that is complementary to its target transcription product. The nucleic acid can comprise DNA, RNA or a chemical analog, that binds to the messenger RNA produced by the target gene. Binding of the antisense nucleic acid prevents translation and thereby inhibits or reduces target protein expression. Antisense nucleic acid molecules may be chemically synthesized using naturally occurring nucleotides or variously modified nucleotides designed to increase the biological stability of the molecules or to increase the physical stability of the duplex formed with mRNA or the native gene e.g. phosphorothioate derivatives and acridine substituted nucleotides. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced.

The term “siRNA” refers to a short inhibitory RNA that can be used to silence gene expression of a specific gene. The siRNA can be a short RNA hairpin (e.g. shRNA) that activates a cellular degradation pathway directed at mRNAs corresponding to the siRNA. Methods of designing specific siRNA molecules or shRNA molecules and administering them are known to a person skilled in the art. It is known in the art that efficient silencing is obtained with siRNA duplex complexes paired to have a two nucleotide 3′ overhang. Adding two thymidine nucleotides is thought to add nuclease resistance. A person skilled in the art will recognize that other nucleotides can also be added.

Aptamers are short strands of nucleic acids that can adopt highly specific 3-dimensional conformations. Aptamers can exhibit high binding affinity and specificity to a target molecule. These properties allow such molecules to specifically inhibit the functional activity of proteins and are included as agents that inhibit Notch, BMP, Bet domain protein, G9a and EZH2.

The term “antibody” as used herein is intended to include monoclonal antibodies, polyclonal antibodies, and chimeric antibodies. The antibody may be from recombinant sources and/or produced in transgenic animals. The term “antibody fragment” as used herein is intended to include without limitations Fab, Fab′, F(ab′)2, scFv, dsFv, ds-scFv, dimers, minibodies, diabodies, and multimers thereof, multispecific antibody fragments and Domain Antibodies. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques.

Antibodies to such proteins may be prepared using techniques known in the art such as those described by Kohler and Milstein, Nature 256, 495 (1975) and in U.S. Pat. Nos. RE 32,011; 4,902,614; 4,543,439; and 4,411,993, which are incorporated herein by reference. (See also Monoclonal Antibodies, Hybridomas: A New Dimension in Biological Analyses, Plenum Press, Kennett, McKearn, and Bechtol (eds.), 1980, and Antibodies: A Laboratory Manual, Harlow and Lane (eds.), Cold Spring Harbor Laboratory Press, 1988, which are also incorporated herein by reference). Within the context of the present disclosure, antibodies are understood to include monoclonal antibodies, polyclonal antibodies, antibody fragments (e.g., Fab, and F(ab′)₂) and recombinantly produced binding partners.

For producing polyclonal antibodies a host, such as a rabbit or goat, is immunized with the immunogen or immunogen fragment, generally with an adjuvant and, if necessary, coupled to a carrier; antibodies to the immunogen are collected from the sera. Further, the polyclonal antibody can be absorbed such that it is monospecific. That is, the sera can be absorbed against related immunogens so that no cross-reactive antibodies remain in the sera rendering it monospecific.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art, (e.g., the hybridoma technique originally developed by Kohler and Milstein (Continuous cultures of fused cells secreting antibody of predefined specificity. Nature 256:495-497, 1975) as well as other techniques such as the human B-cell hybridoma technique (Kozbor, D, and Roder, J: The production of monoclonal antibodies from human lymphocytes. Immunology Today 4:3 72-79, 1983), the EBV-hybridoma technique to produce human monoclonal antibodies (Cole et al. Monoclonal Antibodies in Cancer Therapy (1985) Allen R. Bliss, Inc., pages 77-96) and screening of combinatorial antibody libraries (Huse, W, Sastry, L, Iverson, S, Kang, A, Alting-Mees, M, Burton, D, Benkovic, S, and Lerner, R: Generation of a large combinatorial library of the immunoglobulin repertoire in phage lambda. Science 246:4935 1275-1282, 1989). Hybridoma cells can be screened immunochemically for production of antibodies specifically reactive with the protein or fragment thereof and the monoclonal antibodies can be isolated.

For producing recombinant antibodies (see generally Huston et al, 1991; Johnson and Bird, 1991; Mernaugh and Mernaugh, 1995), messenger RNAs from antibody producing B-lymphocytes of animals, or hybridoma are reverse-transcribed to obtain complementary DNAs (cDNAs). Antibody cDNA, which can be full or partial length, is amplified and cloned into a phage or a plasmid. The cDNA can be a partial length of heavy and light chain cDNA, separated or connected by a linker. The antibody, or antibody fragment, is expressed using a suitable expression system to obtain recombinant antibody. Antibody cDNA can also be obtained by screening pertinent expression libraries.

Chimeric antibody derivatives, i.e., antibody molecules that combine a non-human animal variable region and a human constant region are also contemplated within the scope of the disclosure. Chimeric antibody molecules can include, for example, the antigen binding domain from an antibody of a mouse, rat, or other species, with human constant regions. Conventional methods may be used to make chimeric antibodies containing the immunoglobulin variable region which recognizes the target (See, for example, Morrison et al. (Chimeric Human Antibody Molecules: Mouse Antigen-Binding Domains with Human Constant Region Domains. PNAS 81:21 6851-6855, 1984), and Takeda et al. (Construction of chimaeric processed immunoglobulin genes containing mouse variable and human constant region sequences. Nature 314:452-454), and the patents of Cabilly et al., U.S. Pat. No. 4,816,567; Boss et al., U.S. Pat. No. 4,816,397; Tanaguchi et al., European Patent Publication EP171496; European Patent Publication 0173494, United Kingdom patent GB 2177096B).

Monoclonal or chimeric antibodies specifically reactive with a target as described herein can be further humanized by producing human constant region chimeras, in which parts of the variable regions, particularly the conserved framework regions of the antigen-binding domain, are of human origin and only the hypervariable regions are of non-human origin. Such immunoglobulin molecules may be made by techniques known in the art, (e.g., Teng et al. (Construction and Testing of Mouse—Human Heteromyelomas for Human Monoclonal Antibody Production. PNAS 80:12 7308-7312, 1983), Kozbor et al., supra; Olsson et al. (Methods in Enzymol, 92:3-16 1982) and PCT Publication WO92/06193 or EP 0239400). Humanized antibodies can also be commercially produced (Scotgen Limited, 2 Holly Road, Twickenham, Middlesex, Great Britain.)

The inhibitors described herein may also contain or be used to obtain or design “peptide mimetics”. For example, a peptide mimetic may be made to mimic the function of an inhibitor. “Peptide mimetics” are structures which serve as substitutes for peptides in interactions between molecules (See Morgan et al (1989), Ann. Reports Med. Chem. 24:243-252 for a review). Peptide mimetics include synthetic structures which may or may not contain amino acids and/or peptide bonds but retain the structural and functional features. Peptide mimetics also include molecules incorporating peptides into larger molecules with other functional elements (e.g., as described in WO 99/25044). Peptide mimetics also include peptoids, oligopeptoids (Simon et al (1972) Proc. Natl. Acad, Sci USA 89:9367) and peptide libraries containing peptides of a designed length representing all possible sequences of amino acids corresponding to an inhibitor peptide disclosed herein.

Peptide mimetics may be designed based on information obtained by systematic replacement of L-amino acids by D-amino acids, replacement of side chains with groups having different electronic properties, and by systematic replacement of peptide bonds with amide bond replacements. Local conformational constraints can also be introduced to determine conformational requirements for activity of a candidate peptide mimetic. The mimetics may include isosteric amide bonds, or D-amino acids to stabilize or promote reverse turn conformations and to help stabilize the molecule. Cyclic amino acid analogues may be used to constrain amino acid residues to particular conformational states. The mimetics can also include mimics of the secondary structures of the proteins described herein. These structures can model the 3-dimensional orientation of amino acid residues into the known secondary conformations of proteins. Peptoids may also be used which are oligomers of N-substituted amino acids and can be used as motifs for the generation of chemically diverse libraries of novel molecules.

The term “subject” as used herein refers to any member of the animal kingdom, optionally, a human. In an embodiment, the subject is human.

Also provided herein is a method of determining the type of rhabdoid tumor of a sample comprising:

a) (i) determining a sample gene expression profile and/or (ii) a sample methylation profile from DNA from the sample, said sample gene expression profile and/or sample methylation profile comprising the level of gene expression and/or methylation, respectively, of at least three, optionally at least 5, at least 7, at least 10, at least 15, at least 20 or all of the genes listed in Table 3 or Table 5;

b) determining the level of similarity of said sample gene expression profile and/or sample methylation profile to one or more control profiles, wherein
(i) a high level of similarity of the sample profile to a Group 1 specific control profile; a low level of similarity to a Group 2A or Group 2B control profile indicates that the sample is a Group 1 rhabdoid tumor; or a higher level of similarity to a Group 1 specific control profile than to a Group 2A or Group 2B control profile indicates the sample is a Group 1 type rhabdoid tumor;
(ii) a high level of similarity of the sample profile to a Group 2A specific control profile; a low level of similarity to a Group 2B or Group 1 control profile indicates that the sample is a Group 2A rhabdoid tumor; or a higher level of similarity to a Group 2A specific control profile than to a Group 2B or Group 1 control profile indicates the sample is a Group 2A type rhabdoid tumor; or
(iii) a high level of similarity of the sample profile to a Group 2B specific control profile; a low level of similarity to a Group 2A or Group 1 control profile indicates that the sample is a Group 2B rhabdoid tumor; or a higher level of similarity to a Group 2B specific control profile than to a Group 2A or Group 1 control profile indicates the sample is a Group 2B type rhabdoid tumor.

In an embodiment, a) comprises (i) determining the sample gene expression profile and (ii) determining the sample methylation profile.

As used herein, “methylation” refers specifically to DNA methylation, and more particularly to a modification in which a methyl group or hydroxymethyl group is added to the 5 position of a cytosine residue to form a 5-methyl cytosine (5-mCyt) or 5-hydroxymethylcytosine (5-hmC).

As used herein, “CpG locus” or “methylation locus” refers to an individual CpG dinucleotide sequence in genomic DNA which is capable of being methylated. Individual CpG loci may be identified by reference to an Illumina CpG locus (Illumina ID #) which is defined by a chromosome number, genomic coordinate (referenced to NCBI, hg19), genome build (37), and +/−strand designation to unambiguously define each CpG locus. The genomic information is publically available through the UCSC genome browser at https://genome.ucsc.edu/.

The term “methylation level” refers to a measure of the amount of methylation at a target site (for example, a CpG locus or a number of CpG loci in a gene) within a DNA molecule in a sample. For example, the level of methylation can be measured for one or more CpG dinucleotides, or for a region of DNA. If the methylation level of a target site within a sample is higher than a reference level, the sample is considered to have increased methylation relative to the reference at the target site. Conversely, if the methylation level of a target site within a sample is lower than the reference level, the sample is considered to have a decreased methylation level relative to the reference at the target site. The target site may be an individual CpG locus or a region of DNA comprising multiple CpG loci, for example, a gene promoter. Methylation levels of a target site may be measured by methods known in the art, for example, as a “Is value” or “beta value”, which is calculated as:

β value=intensity of the methylated target(M)/(intensity of the unmethylated target(U)+intensity of the methylated target(M)+100)

A β value of zero indicates no methylation and a value of one indicates 100% methylation.

As used herein the term “gene” refers to a genomic DNA sequence that comprises a coding sequence associated with the production of a polypeptide or polynucleotide product (e.g., rRNA, tRNA). The methylation level of a gene as used herein, encompasses the methylation level of sequences which are known or predicted to affect expression of the gene, including the promoter, enhancer, and transcription factor binding sites. As used herein, the term “enhancer” refers to a cis-acting region of DNA that is located up to 1 Mbp (upstream or downstream) of a gene.

As used herein, the term “sample methylation profile” or “sample profile” refers to the methylation levels at one or more target sequences in a subject's genomic DNA. The target sequence may be an individual CpG locus or a region of DNA comprising multiple CpG loci, for example, a gene promoter or CpG island. The methylation profile of a sample tested according to the methods disclosed herein is referred to as a sample profile.

The sample methylation profile is compared to one or more control profiles. The control profile may be a reference value and/or may be derived from one or more samples, optionally from historical methylation data for a patient or pool of patients who are known to have, or not have, Group 1, Group 2A or Group 2B rhabdoid tumors. In such cases, the historical methylation data can be a value that is continually updated as further samples are collected and individuals are identified. It will be understood that the control profile represents an average of the methylation levels for selected genes as described herein. Average methylation values may, for example, be the mean values or median values.

For example, a “Group 1 specific control profile” or “Group 1 control profile” may be generated by measuring the methylation levels at specified target sequences in genomic DNA from an individual subject, or population of subjects, who are known to have a Group 1 rhabdoid tumor. Similarly, a “Group 2, 2A or 2B control profile” may be generated by measuring the methylation levels at specified target sequences in genomic DNA from an individual subject or population of subjects who are known to have a Group 2, 2A or 2B rhabdoid tumor.

As used herein, the phrase “detecting and/or screening” for a condition refers to a method or process of determining if a subject has or does not have said condition.

Methods of DNA methylation profiling of target genomic regions are generally known in the art (Stevens et al 2013, Harris et al 2010 and Hirst 2013).

For example, a non-limiting list of exemplary methods that may be used to determine methylation levels at a specified target sequence of DNA include: bisulfite sequencing, pyrosequencing, methylation-sensitive single-strand conformation analysis (MS-SSCA), high resolution melting analysis (HRM), methylation-sensitive single nucleotide primer extension (MS-SnuPE), base-specific cleavage/MALDI-TOF, methylation-specific PCR (MSP), methylation-sensitive restriction enzyme-based methods and/or microarray-based methods.

Accordingly, in one embodiment, determining the sample methylation profile in a)ii) comprises the steps:

a) providing the sample comprising genomic DNA from the subject;

b) optionally, isolating DNA from the sample;

c) optionally, treating DNA from the sample with bisulfite for a time and under conditions sufficient to convert non-methylated cytosines to uracils;

d) optionally, amplifying the DNA; and

e) determining the methylation status at the selected genes by means of bisulfite sequencing, pyrosequencing, methylation-sensitive single-strand conformation analysis (MS-SSCA), high resolution melting analysis (HRM), combined bisulfite restriction analysis (COBRA), methylation-sensitive single nucleotide primer extension (MS-SnuPE), base-specific cleavage/MALDI-TOF, methylation-specific PCR (MSP), methylation-sensitive restriction enzyme-based methods, microarray-based methods, whole-genome bisulfite sequencing (WGBS, MethylC-seq or BS-seq), reduced-representation bisulfite sequencing(RRBS), and/or enrichment-based methods such as MeDIP-seq, MBD-seq, or MRE-seq.

As used herein, the term “treating DNA from the sample with bisulfite” refers to treatment of DNA with a reagent comprising bisulfite, disulfite, hydrogen sulfite or combinations thereof, for a time and under conditions sufficient to convert unmethylated DNA cytosine residues to uracil, thereby facilitating the identification of methylated and unmethylated CpG dinucleotide sequences. Bisulfite modifications to DNA may be detected according to methods known in the art, for example, using sequencing or detection probes which are capable of discerning the presence of a cytosine or uracil residue at the CpG site.

Methods of determining the similarity between methylation profiles are well known in the art. Methods of determining similarity may in some embodiments provide a non-quantitative measure of similarity, for example, using visual clustering. In another embodiment, similarity may be determined using methods which provide a quantitative measure of similarity.

For example, in an embodiment, similarity may be measured using hierarchical clustering, optionally using Manhattan distance. For example, unsupervised hierarchical clustering of a sample with a Group 1 specific control profile indicates similarity to the Group 1 specific control profile. Likewise, unsupervised hierarchical clustering of a sample with a Group 2 control profile indicates similarity to the Group 2 control profile.

In another embodiment, similarity may be measured by computing a “correlation coefficient”, which is a measure of the interdependence of random variables that ranges in value from −1 to +1, indicating perfect negative correlation at −1, absence of correlation at zero, and perfect positive correlation at +1. In an embodiment, the correlation coefficient may be a linear correlation coefficient, for example, a Pearson product-moment correlation coefficient.

A Pearson correlation coefficient (r) is calculated using the following formula:

$$r = \frac{{\sum }_{i} ( {x}_{i} - \overline{x} ) ( {𝓎}_{i} - \overline{𝓎} )}{\sqrt{{\sum }_{i} {( {x}_{i} - \overline{x} )}^{2}} \sqrt{{\sum }_{i} {( {𝓎}_{i} - \overline{𝓎} )}^{2}}}$$

In one embodiment, x and y are the beta values for various CpG loci in a sample profile and a control profile, respectively.

In an embodiment, a high level of similarity to the control profile is indicated by a correlation coefficient between the sample profile and the control profile having an absolute value between 0.5 to 1, optionally between 0.75 to 1, and a low level of similarity to the control profile is indicated by a correlation coefficient between the sample profile and the control profile having an absolute value between 0 to 0.5, optionally between 0 to 0.25.

It will be appreciated that any “correlation value” which provides a quantitative scaling measure of similarity between methylation profiles may be used to measure similarity.

In an embodiment, determining the sample gene expression profile comprises measuring the expression level of the gene in the sample.

The term “difference in the level of expression” refers to an increase or decrease in the measurable expression level of a gene as compared with the measurable expression level of the same gene in a second sample or control. In one embodiment, the differential expression can be compared using the ratio of the level of expression of the first sample as compared with the expression level of the second sample or control, wherein the ratio is not equal to 1.0. For example, a gene is differentially expressed if the ratio of the level of expression in a first sample as compared with a second sample is greater than or less than 1.0. For example, a ratio of greater than 1, 1.2, 1.5, 1.7, 2, 3, 5, 10, 15, 20 or more, ora ratio less than 1, 0.8, 0.6, 0.4, 0.2, 0.1, 0.05, 0.001 or less. In another embodiment the differential expression is measured using p-value. For instance, when using p-value, a gene is identified as being differentially expressed as between a first and second population when the p-value is less than 0.1, preferably less than 0.05, more preferably less than 0.01, even more preferably less than 0.005, the most preferably less than 0.001.

“Determining the expression of” a gene can be readily accomplished by a person skilled in the art. In one embodiment, a probe that hybridizes to the mRNA sequence of the gene's nucleic acid sequence as can be used to detect and quantify the amount of the mRNA in the sample.

A nucleotide probe may be labelled with a detectable marker such as a radioactive label which provides for an adequate signal and has sufficient half life such as 32P, 3H, 14C or the like. An appropriate label may be selected having regard to the rate of hybridization and binding of the probe to the nucleotide to be detected and the amount of nucleotide available for hybridization.

The term “hybridize” refers to the sequence specific non-covalent binding interaction with a complementary nucleic acid. Appropriate stringency conditions which promote hybridization are known to those skilled in the art, or can be found in Current Protocols in Molecular Biology, John Wiley & Sons, N.Y. (1989), 6.3.1 6.3.6. For example, 6.0×sodium chloride/sodium citrate (SSC) at about 45° C. for 15 minutes, followed by a wash of 2.0×SSC at 50° C. for 15 minutes may be employed.

The stringency may be selected based on the conditions used in the wash step. For example, the salt concentration in the wash step can be selected from a high stringency of about 0.2×SSC at 50° C. for 15 minutes. In addition, the temperature in the wash step can be at high stringency conditions, at about 65° C. for 15 minutes.

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.−16.6(Log10 [Na+])+0.41(%(G+C)−600/I), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% sequence identity, the final wash temperature will be reduced by about 5° C. Based on these considerations those skilled in the art will be able to readily select appropriate hybridization conditions. In an embodiment, stringent hybridization conditions are selected. By way of example the following conditions may be employed to achieve stringent hybridization: hybridization at 5× sodium chloride/sodium citrate (SSC)/5× Denhardt's solution/1.0% SDS at Tm −5° C. based on the above equation, followed by a wash of 0.2×SSC/0.1% SDS at 60° C. for 15 minutes. Moderately stringent hybridization conditions include a washing step in 3× SSC at 42° C. for 15 minutes. It is understood, however, that equivalent stringencies may be achieved using alternative buffers, salts and temperatures. Additional guidance regarding hybridization conditions may be found in: Current Protocols in Molecular Biology, John Wiley & Sons, N.Y., 1989, 6.3.1-6.3.6 and in: Sambrook et al., Molecular Cloning, a Laboratory Manual, Cold Spring Harbor Laboratory Press, 2000, Third Edition.

In another embodiment, primers that are able to amplify the gene sequence can be used, for example, in a quantitative PCR assay to determine the expression level of the gene.

As used herein, the term “amplify”, “amplifying” or “amplification” of DNA refers to the process of generating at least one copy of a DNA molecule or portion thereof. Methods of amplification of DNA are well known in the art, including but not limited to polymerase chain reaction (PCR), ligase chain reaction (LCR), self-sustained sequence replication (3SR), nucleic acid sequence based amplification (NASBA), strand displacement amplification (SDA), multiple displacement amplification (MDA) and rolling circle amplification (RCA).

The length and bases of primers for use in a PCR are selected so that they will hybridize to different strands of the desired sequence and at relative positions along the sequence such that an extension product synthesized from one primer when it is separated from its template can serve as a template for extension of the other primer into a nucleic acid of defined length. Primers which may be used in the disclosure are oligonucleotides, i.e., molecules containing two or more deoxyribonucleotides of the nucleic acid molecules of the disclosure which occur naturally as in a purified restriction endonuclease digest or are produced synthetically using techniques known in the art such as for example phosphotriester and phosphodiester methods (See Good et al. Nucl. Acid Res 4:2157, 1977) or automated techniques (See for example, Conolly, B.A. Nucleic Acids Res. 15:15(7): 3131, 1987). The primers are capable of acting as a point of initiation of synthesis when placed under conditions which permit the synthesis of a primer extension product which is complementary to a DNA sequence of the disclosure, i.e., in the presence of nucleotide substrates, an agent for polymerization such as DNA polymerase and at suitable temperature and pH. Preferably, the primers are sequences that do not form secondary structures by base pairing with other copies of the primer or sequences that form a hairpin configuration. The primer optionally comprises between about 7 and 25 nucleotides.

The primers may be labelled with detectable markers which allow for detection of the amplified products. Suitable detectable markers are radioactive markers such as P-32, S-35, I-125, and H-3, luminescent markers such as chemiluminescent markers, preferably luminol, and fluorescent markers, preferably dansyl chloride, fluorcein-5-isothiocyanate, and 4-fluor-7-nitrobenz-2-axa-1,3 diazole, enzyme markers such as horseradish peroxidase, alkaline phosphatase, β-galactosidase, acetylcholinesterase, or biotin.

It will be appreciated that the primers may contain non-complementary sequences provided that a sufficient amount of the primer contains a sequence which is complementary to a nucleic acid molecule of the disclosure or oligonucleotide fragment thereof, which is to be amplified. Restriction site linkers may also be incorporated into the primers allowing for digestion of the amplified products with the appropriate restriction enzymes facilitating cloning and sequencing of the amplified product.

As used herein the term “sample” refers to a biological sample comprising genomic DNA from a human subject. The sample may, for example, comprise tumor tissue (biopsy) or blood containing circulating tumor cells.

Confirmation of a diagnosis of a particular type of rhabdoid tumor aids in medical management by enabling targeted therapeutic treatment.

Accordingly, in a further embodiment, the method comprises treating the subject with an inhibitor of Notch or an inhibitor of an epigenomic regulator if the sample is typed as a Group 1 rhabdoid tumor or treating the subject with an inhibitor of BMP or PDGFRβ signaling if the sample is typed as a Group 2A or Group 2B rhabdoid tumor.

Another aspect provides a kit for determining the type of rhabdoid tumor in a sample, comprising:
(a) at least one detection agent for determining the gene expression level and/or methylation level of:
   at least 3, optionally at least 5, at least 7, at least 10, at least 15, at least 20 or all the genes from Table 3 or Table 5; and
(b) instructions for use.

In an embodiment, the kit further comprises bisulfite conversion reagents, methylation-dependent restriction enzymes, methylation-sensitive restriction enzymes, PCR reagents, probes and/or primers.

In another embodiment, the kit further comprises a computer-readable medium that causes a computer to compare gene expression and/or methylation levels from a sample at the selected genes to one or more control profiles and compute a correlation value between the sample and control profile.

The above disclosure generally describes the present application. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

### Results

### WGS Analyses Reveal Predominant Structural Coding Events in ATRTs

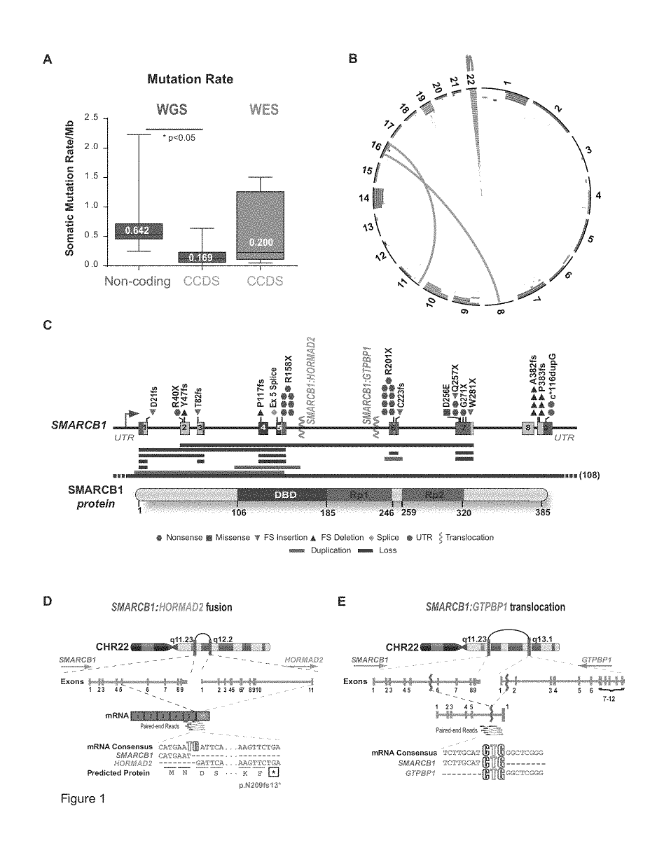

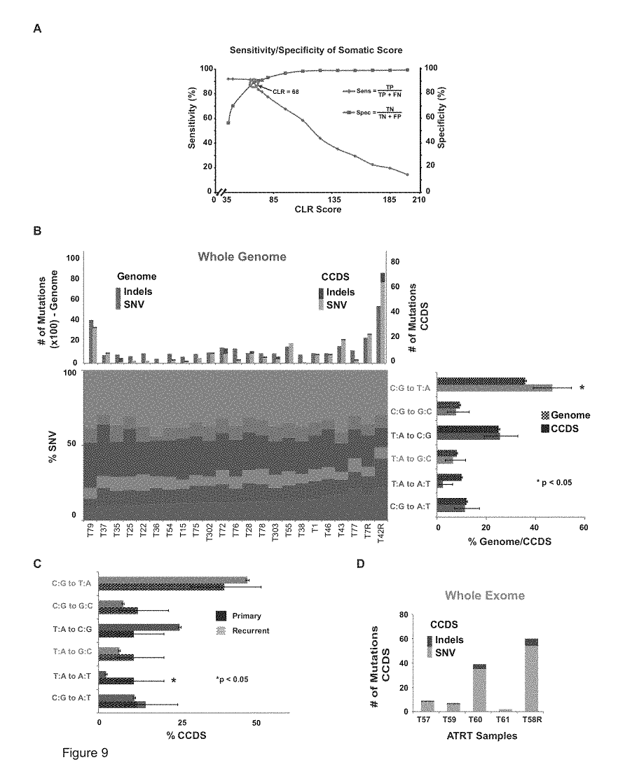

The present inventors combined whole-genome (WGS)/exome sequencing (WES) to characterize 50 ATRTs (47 primary and 3 recurrent tumors). WGS was performed on 21 primary normal/tumor pairs, and WES on an additional 5 normal/tumor pairs and 22 unmatched tumors; high resolution copy number profiles were generated for 60 ATRTs (OmniQuad, Illumine). Identification and validation of single nucleotide substitutions (SNVs), small insertion/deletion (indels) and somatic copy number alterations (SCNAs) were performed as described in methods. Consistent with prior exome studies, the WGS/WES analyses revealed an overall low coding region mutation rate in primary ATRT (0.00 to 1.01 SNV and indels/Mb; FIG. 9). Interestingly, however the analyses indicated significantly higher whole genome somatic mutation rates (0.64 mutation/Mb) (FIG. 1A) and suggest the scope and frequency of pathogenic genomic alterations in ATRT is underestimated by exome analyses. In order to identify and enumerate potential pathogenic somatic copy number alterations (SCNAs) and other structural genomic events in ATRTs, the present inventors integrated WGS/WES/RNAseq as well as copy number analyses using ultra-high resolution SNP and 450K methylation array data as described in methods. These analyses of 180 distinct tumors identified a spectrum of 379 SCNAs which included whole arm gains and losses, focal deletions, duplications and notably uncovered novel complex gene re-arrangements associated with inter- and intra-chromosomal translocations (FIG. 1B). Interestingly, analyses of WGS data (21 tumors) as well as SNP and/or 450K methylation data (162 tumors) revealed ATRT coding regions have 1.84-3.57 structural alterations/tumor and indicate a higher incidence of somatic genomic alterations in ATRTs than previously appreciated and indicate somatic structural alterations as unrecognized, important genetic mechanisms in ATRT.

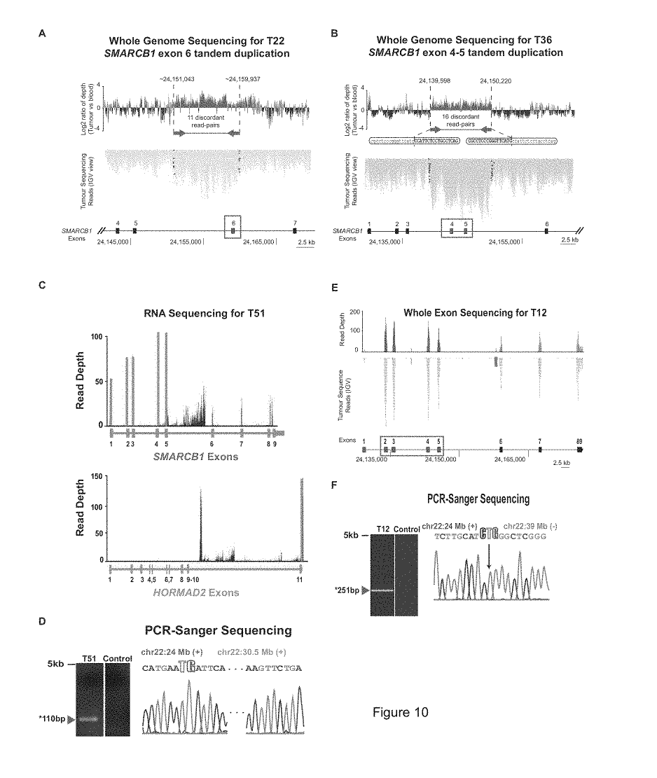

Indeed, although prior studies suggested mutations as major mechanisms for SMARCB1 loss in rhabdoid tumors (Biegel et al., 2002; Eaton et al., 2011; Sévenet et al., 1999), a majority of ATRTs (55.8%) exhibited SMARCB1 loss (FIG. 1C) via various structural mechanisms including novel gene fusions and exon duplications events. In addition to 7 novel mutations including an exon 5 splice acceptor mutation and SMARCB1 fusion to two unrelated coding genes (HORMAD2 and GTPBPI) (FIG. 1D,E; FIG. 10) detected only by RNAseq, homozygous alterations of SMARCA4 were identified in two tumors. This multi-method genetic analyses of the entire SMARCB1 coding region in a large cohort of ATRTs did not reveal any mutational “hot-spots” as previously reported (Bourdeaut et al., 2011; Jackson et al., 2009).

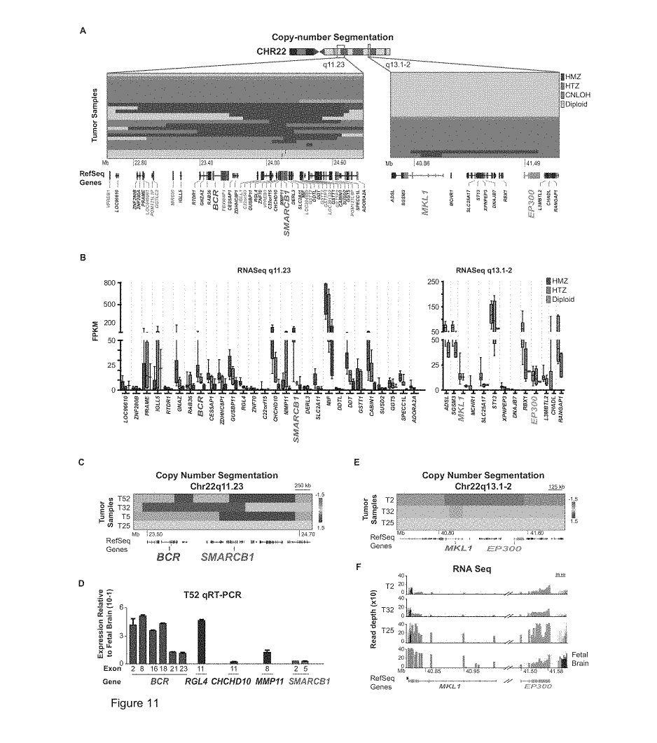

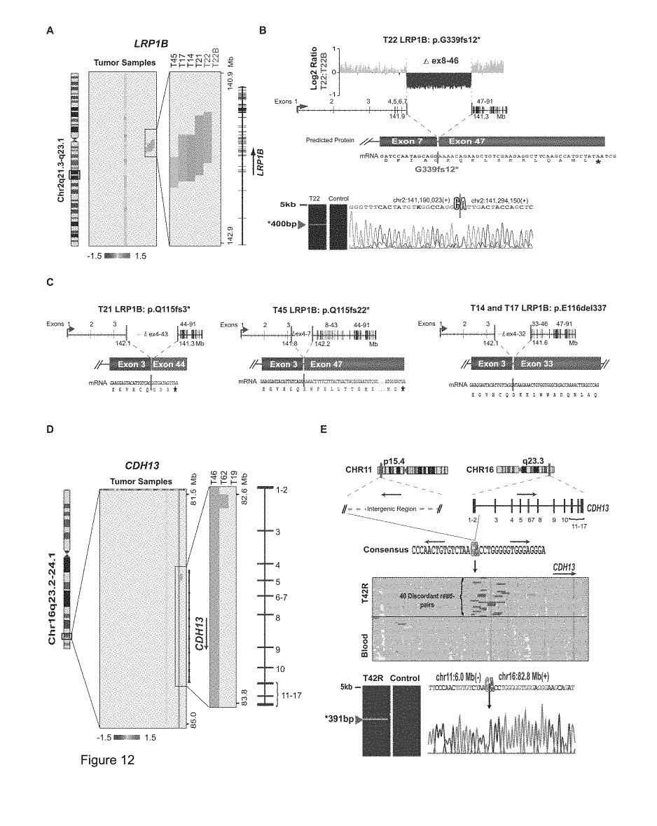

Using segmentation analyses of SNP, methylation array, WGS and RNAseq data, novel recurrent SCNAs were uncovered targeting coding regions in up to 20% of ATRTs that resulted in gene expression changes. Cell adhesion, neural development and chromatin remodelling genes were most frequently affected and included BCR, MKL1 and EP300 on chr 22 which respectively exhibited focal homozygous and heterozygous deletions in 31 (20.6%), 6 (3.3%) and 6 (3.3%) of 180 primary ATRTs analysed (FIG. 11A). Also observed were alterations of KDM3B, NCOR1 (Zhang et al., 2007) and CREB1—an EP300 protein partner (Lonze et al., 2002) and a gene fusion of Histone H3.3 chaperone HIRA. LRP1β (5/180; 2.8%), on chr 2q.21.3, (Cowin et al., 2012; Sonoda et al., 2004) which was the most frequent focal alteration in ATRTs (FIG. 12A). In contrast to large multigene events in other cancers, LRP1β alterations in ATRTs were primarily intragenic heterozygous multi-exon deletion predicted to disrupt LRP1β protein (FIG. 12B,C). Global/focal SCNAs and gene re-arrangements of other chromosomal regions were seen in 1-7% of ATRT (FIG. 12D,E).

Collectively, the integrated genomic interrogation has uncovered multiple novel global and focal alterations that accompanies SMARCB1 loss and highlights additional complexity to the ATRT genome than previously realized from lower resolution analyses. The functions of most recurrently targeted loci in cancer and neural development suggest these are potentially important modifier loci that contribute to diverse phenotypes in tumors initiated by SMARCB1 loss.

ATRTs Comprise 3 Epigenetic Sub-Types with Distinct Clinical Profiles and Genotypes

Although most ATRTs exhibit few recurrent genetic alterations other than SMARCB1, ATRTs were previously observed to comprise at least two transcriptional and clinically distinct sub-types (group 1 and 2 ATRT) (Torchia et al., 2015). Taken together with prior experimental studies, these findings indicate SMARCB1 driven epigenomic mechanisms may be significant determinants of ATRT phenotypes. To investigate sub-group specific therapies it was first sought to define epigenetic subtypes of ATRTs by performing global methylation analyses on 162 primary tumors. Unsupervised cluster analyses of global methylation profiles (Illumina 450K BeadChip methylation arrays) with orthogonal algorithms revealed three distinct methylation classes of ATRTs (FIG. 2A,B; FIG. 13) that exhibited remarkable concordance with transcriptional sub-types determined from gene expression profiles (flumina human HT-12) of an overlapping 90 primary ATRTs (Rand index=0.6129). While group 1 ATRT with neurogenic transcriptomes clustered as a single group in methylation analyses, group 2 ATRTs with mesenchymal transcriptomes further segregated into two methylation subtypes (called group 2A and 2B). ATRT sub-types exhibited distinct clinical and genotypic features (FIG. 2C,D); group 1 and 2A ATRTs respectively correlated with predominant supratentorial/cerebral (37/51; 72.5%), and infratentorial brain (cerebellum, brain stem) (42/64; 65.6%) locations. While group 1 ATRTs were seen in the oldest children (median age 24 months, range 3-164 months; 95% CI 20.70-26.55), group 2A patients were the youngest (median age 12 months, range of 0-240 months; 95% CI 11.05-13.00). Group 2B ATRTs were the most clinically heterogenous and comprised of infra- (9/34; 26.5%), supra-tentorial tumors (17/34; 50.0%) as well as spinal (8/34; 23.5%) tumors which were exclusively restricted to group 2B. They also spanned a broader age range with a bimodal age distribution and the greatest proportion of patients >3yrs of age (12/32; 37.5%). No significant association of gender or incidence of metastases with ATRT molecular subgroups were found.

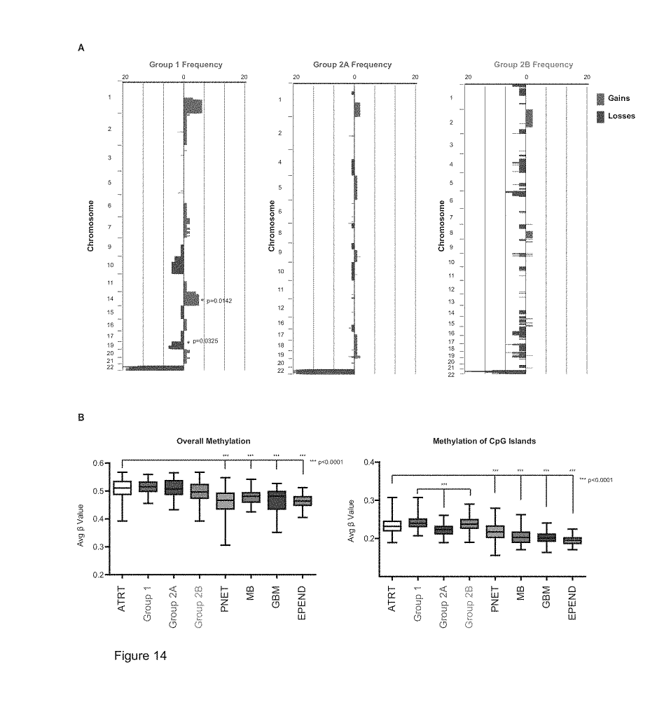

As the WGS and global copy number studies indicated diverse mechanisms of SMARCB1 loss as well as additional recurrent genomic alterations in ATRT, whether ATRT molecular sub-types correlated with genome mutational load, global and SMARCB1 specific genotypes was also investigated. Although the rate of SNV alterations were comparable across ATRT molecular sub-groups, significant genotypic differences were observed with group 2B tumors exhibiting significantly more focal genomic alterations (mean=1.83; 95% CI 1.43-2.31 alterations/tumor; p=0.0024; FIG. 2C) than both group 1 (mean=0.86; 95% CI 0.65-1.12) alterations/tumor), and group 2A (mean=0.88; 95% CI 0.68-1.13 alterations/tumor) tumors. Interestingly, while group 1 ATRTs were distinguished by recurrent chr14 gain and loss of chr19, group 2B tumor genomes exhibited focal copy number losses across multiple chromosomes, group 2 ATRTs were relatively silent (FIG. 14A).

The analyses also revealed that the type of genetic event leading to SMARCB1 loss differed significantly between molecular sub-groups of ATRT (p=2.79×10⁻⁴; FIG. 2C). A majority of group 1 tumors (30/45; 66.7%) exhibited focal/subgenic alterations with predicted retention of the SMARCB1 transcriptional start site; while group 2B tumors had large deletions that encompassed all of SMARCBI, and frequently additional chr 22 genes. Taken together, these findings indicate a novel SMARCBI genotype:phenotype correlation.

ATRT Sub-Groups have Distinct Lineage Enriched Functional Genomes

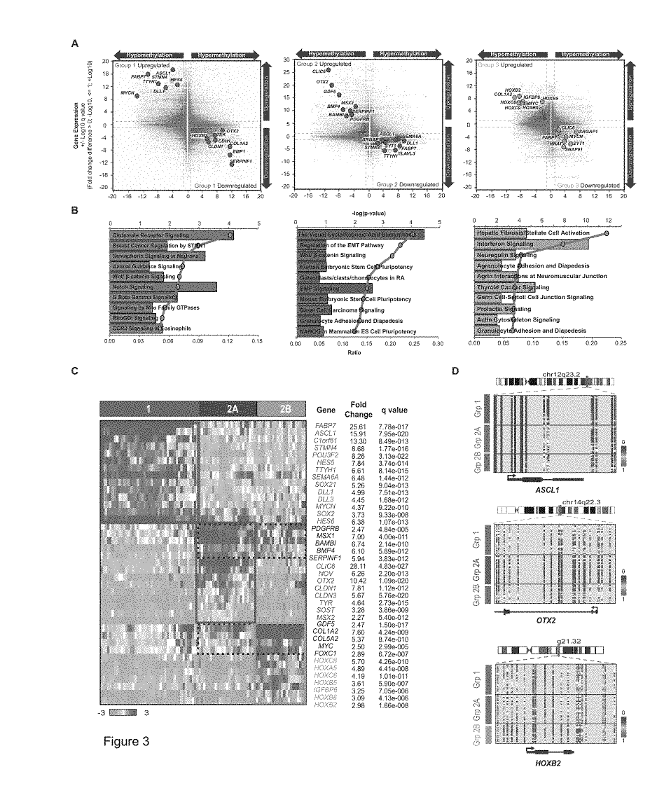

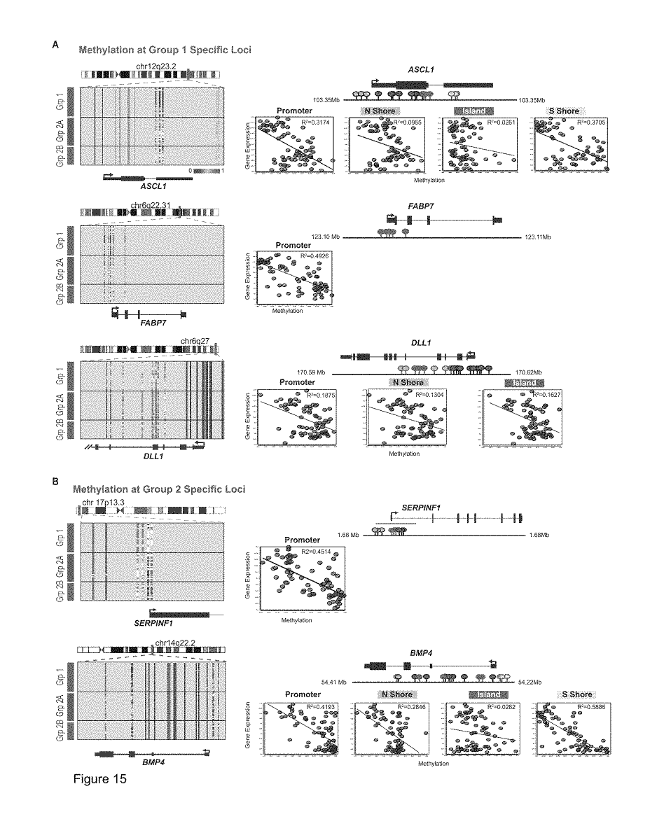

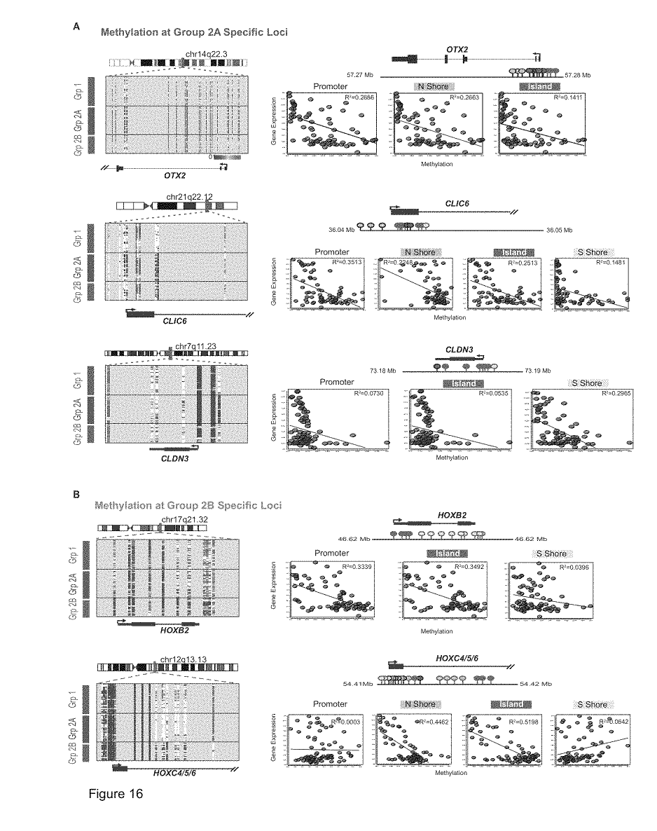

The observation of distinct sub-group specific genotypes suggest SMARCB1 loss may be associated with different functional consequences in molecular sub-types of ATRTs. To define core molecular and cellular features of ATRT sub-groups, supervised analyses of transcriptional and methylation data were integrated which revealed that genes involved in lineage determination and developmental signaling were amongst the most highly differentially methylated and expressed loci across ATRT sub-groups (FIG. 3A). These findings which indicate distinct cell lineage and signaling features of ATRT sub-groups were further corroborated by Ingenuity Pathway Analyses (IPA) (FIG. 3B). Specifically it was observed that neurogenic genes (FABP7, ASCL1, MYCN, c1orf61) and genes involved in NOTCH (DLL1/3 HES5/6), glutamate receptor (SLC17A8, SLC17A6) and axonal guidance (TUBB2B/3/4A, SEMA6A) signaling, comprised the most highly expressed and hypomethylated genes in group 1 ATRTs. While genes that were most commonly differentially expressed and methylated genes in group 2A and 2B tumors were enriched for functions in BMP signaling (BMP4, BAMBI, GDF5, FOXC1) and mesenchymal differentiation (SERPINF1, CLDN10, FBN2, MSXI, PDGFRβ) (FIG. 3C). Group 2A tumors were further distinguished by enrichment of genes implicated in visual cortex/hind brain development (OTX2), retinol (RBP1/7,RDH5/10) and tyrosine metabolism (TYR) while upregulation of MYC and HOXB/C clusters was seen in Group 2B tumors (FIG. 3C). Detailed gene level analyses showed a tight concordance of methylation patterns at promoter associated CpG residues with specific ATRT sub-types thus suggesting epigenetic regulation of functionally important developmental/cell lineage signaling pathways in ATRTs (FIG. 3D; FIGS. 15 and 16). Interestingly, while many of the genes enriched in Group 2A tumors had functions in pluripotency and regulation of EMT, Group 2B tumors exhibited more heterogeneous signaling profiles with enrichment of Interferon signaling, cell adhesion and cytoskeletal development genes (FIG. 3B). Interestingly, while ATRTs generally exhibited a hypermethylated genome relative to other pediatric brain tumors, group 2A ATRTs had the lowest CpG island methylation levels relative to group 1 and 2B tumors (FIG. 14B).

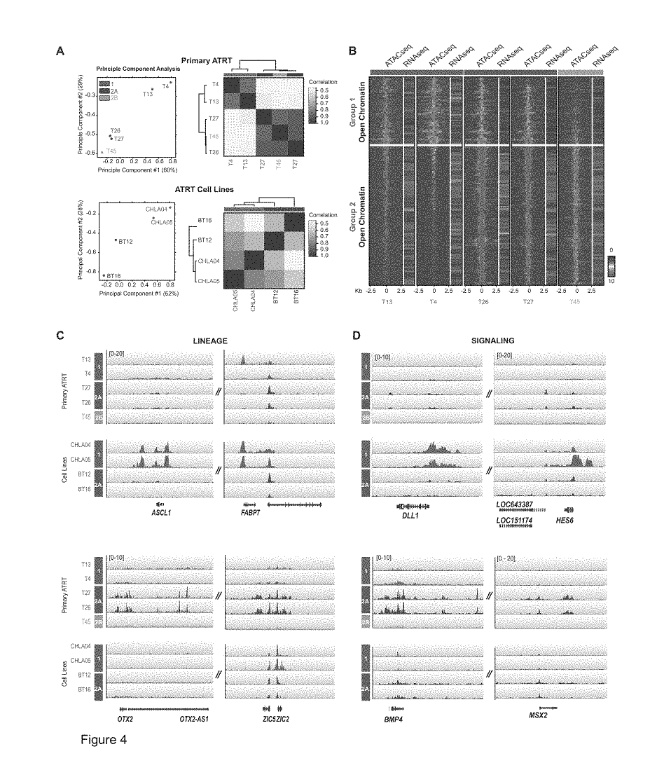

To further investigate the distinct functional epigenome of ATRT sub-groups, high resolution, genome-wide chromatin accessibility mapping was performed using ATAC-seq analyses on 5 primary (two group 1 and 2A, one group 2B ATRTs) and 4 ATRT cell lines. In keeping with global methylation and transcriptional analyses, principle component and correlation analysis of ATAC-seq data showed segregation and association of ATRT sub-types with distinct ATAC-seq profiles (FIG. 4A). Integration of ATAC-seq footprints with RNAseq data revealed remarkably open chromatin landscape in group 2A ATRTs that correlated with generally increased gene expression patterns in contrast to more closed chromatin landscapes and generally decreased gene expression patterns in group 1 tumors, while group 2B ATRTs exhibited an intermediate profile (FIG. 4B). Specifically, neural lineage loci characteristic of group 1 (ASCL1, FABP7) and group 2A/B (OTX2, ZIC1/4, ZIC5/2) ATRTs displayed an open-chromatin state (FIG. 4C; FIGS. 15 and 16). In addition to cell lineage, multiple signaling pathways including ligands of the NOTCH (DLL1, HES6) and BMP (BMP4, MSX2) pathways displayed open-chromatin in a sub-type specific pattern (FIG. 4D). Interestingly, principal and correlation analyses of ATAC-seq data also showed the 4 ATRT cell lines segregated into 2 groups with chromatin openness patterns at cell lineage loci that mirrored those of primary group 1 and 2 ATRTs.

These aggregate data suggest SMARCB1 genotypes and molecular sub-groups of ATRTs are associated with distinct functional epigenomes and indicate epigenomic mechanisms drive lineage specific gene expression as well as activation of potentially therapeutically targetable pathways in ATRTs.

### Pharmacologic Inhibitors of NOTCH and BMP Signaling Abrogate ATRT Cell Growth in a Sub-Group Specific Manner

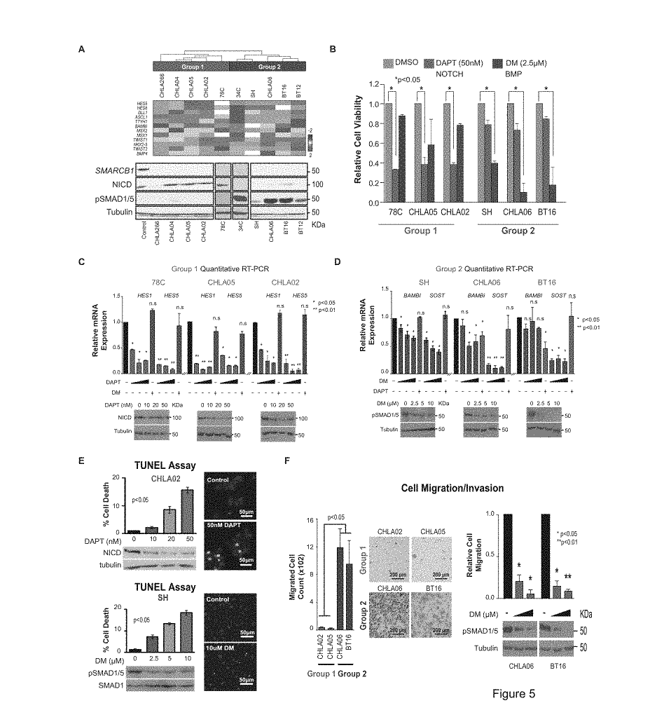

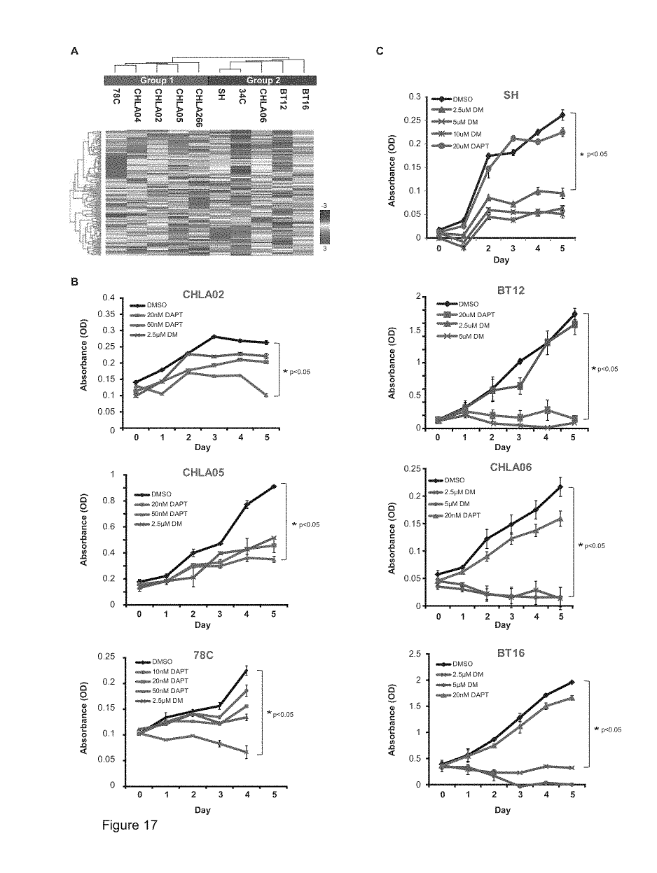

In order to investigate ATRT sub-type specific therapeutics, gene expression profiling was used to determine molecular grouping of a panel of 10 ATRT cell lines—78C and 34C, respectively derived from tumors T13 (group 1), T45 (group 2B) and established lines, CHLA02, CHLA04, CHLA05, CHLA06, CHLA266, BT12, BT16, SH (FIG. 5A; FIG. 17). Supervised analyses of gene expression profiles confirmed group 1 and 2 ATRT cell lines respectively maintained enrichment of neurogenic/NOTCH signaling and mesenchymal/BMP signaling genes seen in corresponding primary tumor subtypes (FIG. 17A). Western blot analyses showed differential expression of NOTCH Intracellular Domain (NICD) and phospho SMAD1/5 (FIG. 5A), respective effectors of the NOTCH and BMP signaling correspondingly in group 1 and 2 cell lines, thus indicating fidelity of sub-type specific signaling pathways in ATRT cell lines.

To evaluate whether NOTCH and BMP signaling maintained in ATRT cell lines are functionally important, the effects of small molecule NOTCH and BMP pathway inhibitors were investigated on phenotypes of group 1 and 2 cell lines. DAPT (N-[N-(3,5-difluorophenacetyl)-L-alanyl]-S-phenylglycine t-butyl ester)—a γ-secretase inhibitor (Geling et al., 2002) and Dorsomorphin (Yu et al., 2008) were used to respectively assess the effects of NOTCH and BMP pathway inhibition on a representative panel of group 1 (78C, CHLA05, CHLA02) and group 2A/B (SH, CHLA06, BT16) ATRT cell lines which showed most consistent robust growth in vitro. Cell viability assays showed treatment with DAPT and DM respectively inhibited group 1 and 2 ATRT cell line growth in a dose dependent manner (FIG. 5B; FIG. 17B). Importantly, cross treatment of group 1 and 2 cell lines respectively with DM and DAPT showed no significant effects on cell growth, thus underscoring sub-group specific activity of these pathways. Western blot and qRT-PCR analyses were used to confirm altered ATRT cell growth induced by DAPT correlated with dose dependent decrease in NICD expression and downregulation of NOTCH transcriptional targets, HES1 and HESS in group 1 cell lines—78C, CHLA02 and CHLA05. Similarly, dose dependent decrease in expression of phospho-SMAD1/5 and corresponding downregulation of BMP target genes SOST and BAMBI in group 2 cell lines—SH, CHLA06, BT16 (FIG. 5C,D; FIG. 17C) were observed. Notably, dose dependent changes in NICD and pSMAD1/5 expression after DAPT and DM treatments correlated with increased cell death as detected by TUNEL assays (FIG. 5E). These findings highlight NOTCH and BMP signaling as important sub-group specific survival pathways in ATRTs.

As group 2 ATRTs have more aggressive clinical phenotypes and exhibit transcriptomes enriched for cell migratory and invasive pathways, whether BMP signaling mediated additional oncogenic phenotypes in ATRT cells was investigated. Transwell migration assays revealed highly invasive and migratory phenotypes in group 2 as compared to group 1 ATRT cell lines. Significantly DM treatment also robustly diminished group 2 ATRT cell invasion and migration in a dose dependent manner with corresponding decreased pSMAD1/5 expression (FIG. 5F). These observations collectively validate NOTCH and BMP signaling as important determinants of sub-group specific cellular phenotypes and also indicate the potential to exploit these lineage-specific signaling dependencies to target ATRT sub-types.

### Epigenetic Regulation of a Novel Enhancer Element Underlies Group 2 ATRT Sensitivity to Small Molecule Inhibitors of PDGFRΔ Signaling

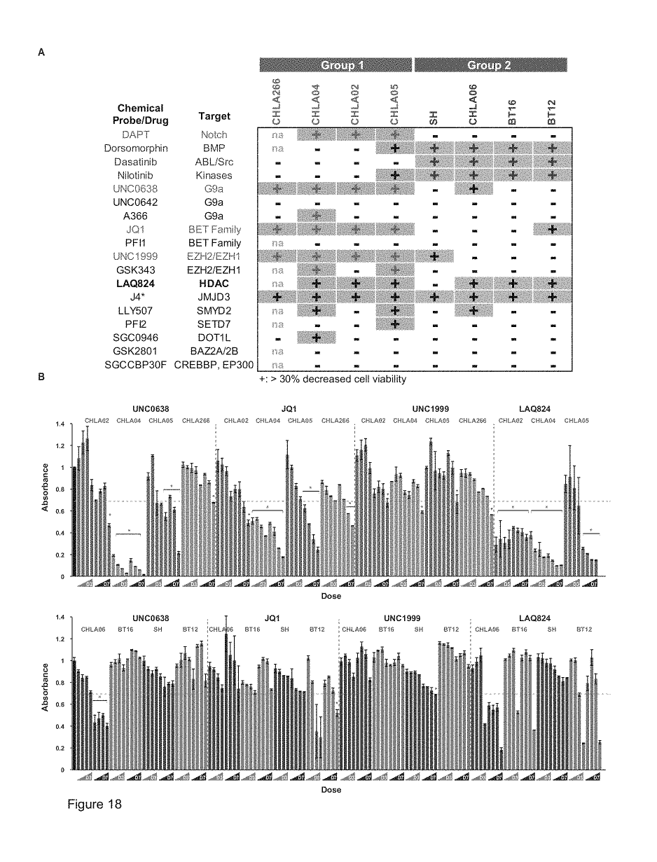

Recent studies have reported promising therapeutic agents targeting a number of epigenetic and signaling pathways in ATRTs (Ginn and Gajjar, 2012), however the relevance of these agents to ATRT sub-types is unknown as these prior studies have examined a very limited number of cell lines. To identify additional ATRT sub-group specific pathways/targets, the effects of 12 small molecule inhibitors/chemical probes targeting epigenetic pathways on growth of 4 group 1 (CHLA266, CHLA04, 02, 05) and 4 group 2 ATRT (SH, CHLA06, BT16 and BT12) cell lines (FIG. 18A) were tested. Small molecule inhibitors were selected with well-defined in vitro cellular activity that target Bromo/BET domain proteins (JQ1, PFI-1, 2, 3, GSK2801, SGC-CBP30), Methyltransferases (GSK343, UNC1999, UNC0642, UNC0638, A-366, LLY507) and histone deacetylases (LAQ824) (FIG. 18A). In addition, therapeutic sensitivity of ATRT cell lines was also tested to Dasatinib and Nilotinib, two related small molecule multi-tyrosine kinase inhibitor as gene expression analyses indicated PDGFRI3, a potential target of these drugs was differentially expressed in ATRT sub-groups (FIG. 3C).

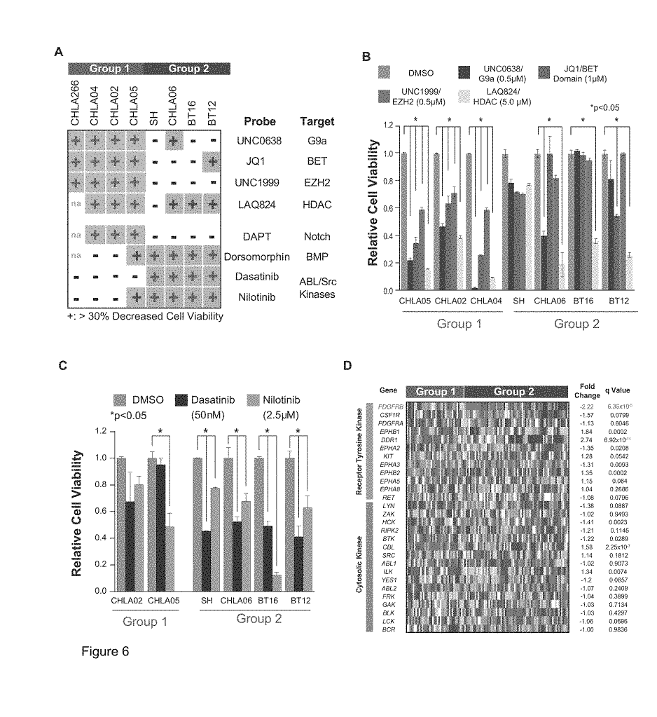

Cell viability/MTS assays performed on ATRT cell lines after treatment with varying doses of epigenetic probes/drugs showed 4 of the 12 small molecules had consistent and significant effects on ATRT cell growth (>30% reduction in cell viability). These included UNC0638 and UNC1999 which respectively target methyltransferases G9a/GLP and EZH2 and JQ1—a BRD4/bromodomain inhibitor and LAQ824—a Histone deactylase inhibitor. Remarkably, while LAQ824 significantly diminished growth of all cell lines, treatment with UNC0638, UNC1999 and JQ1 had sub-group specific effect on ATRT cell growth (FIG. 6A,B; FIG. 18B). All four of the group 1 cell lines exhibited >30% reduction in cell viability upon treatment with varying doses of each of the 3 inhibitors (FIG. 6A,B; FIG. 18). In contrast, 3 of the 4 group 2 cell lines showed no changes in growth after treatment with UNC0638, UNC1999 and JQ1. Interestingly, gene expression analyses show EHMT2 (encodes G9a), EZH2, BRD4 and related loci (BRD1-7) are highly expressed across ATRT sub-types, and suggest therapeutic sensitivity to epigenetic inhibitors may be dependent on the distinct chromatin landscape of ATRT sub-types.

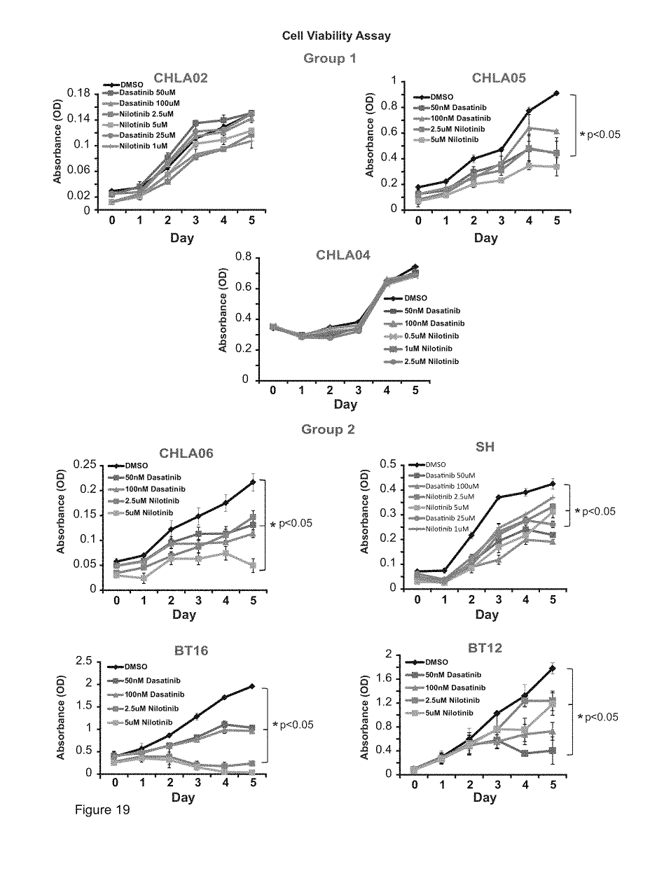

Dasatinib and Nilotinib, are potent related ATP-competitive small-molecule inhibitors of multiple receptor tyrosine kinases (RTKs) including BCR-ABL fusion protein, stem cell factor receptor (c-KIT), platelet-derived growth factor receptor (PDGFR), and Src family kinases (SFKs) (Rix et al., 2007). Both Dasatinib and Nilotinib are widely used in clinical treatment of leukemia (Kantarjian et al., 2006) and successfully repurposed for some adult solid tumors (Araujo and Logothetis, 2010; Schlemmer et al., 2012) but has not been investigated in pediatric brain tumors. Remarkably, in contrast to the relative insensitivity of group 2 ATRTs to epigenetic inhibitors, it was observed that Dasatinib and Nilotinib treatment robustly diminished growth of group 2 but not group 1 ATRT cell lines (FIG. 6C; FIG. 19). The well characterized blood brain permeability and pharmacology of these drugs make them ideal candidates for clinical translation, hence it was sought to further investigate mechanisms underlying the robust effect of both drugs on group 2 ATRT cell growth. Supervised gene expression analyses of all potential tyrosine kinase targets, showed PDGFRβ as most significantly differentially expressed in group 2 versus group 1 ATRT (>2 fold change, p=6.35×10⁻⁵) (FIG. 6D).

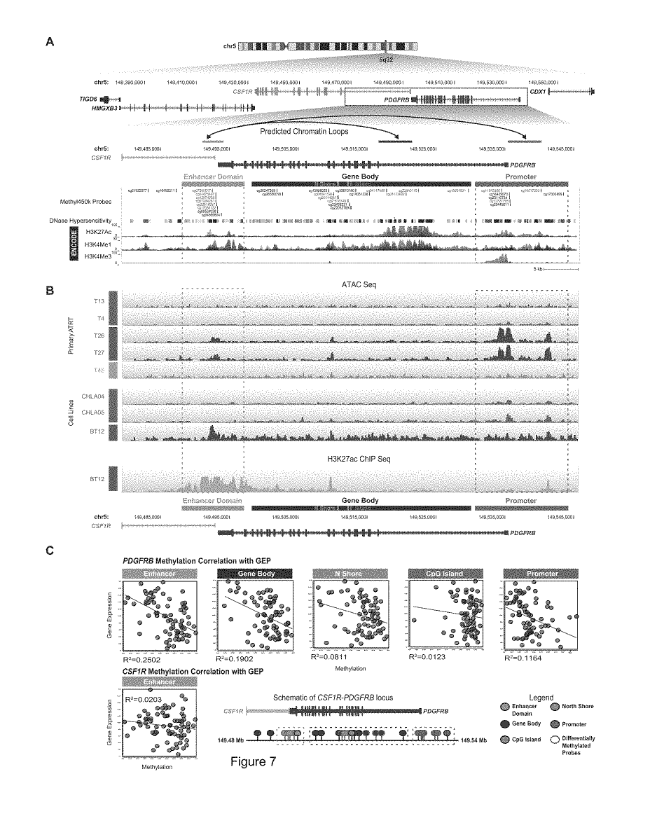

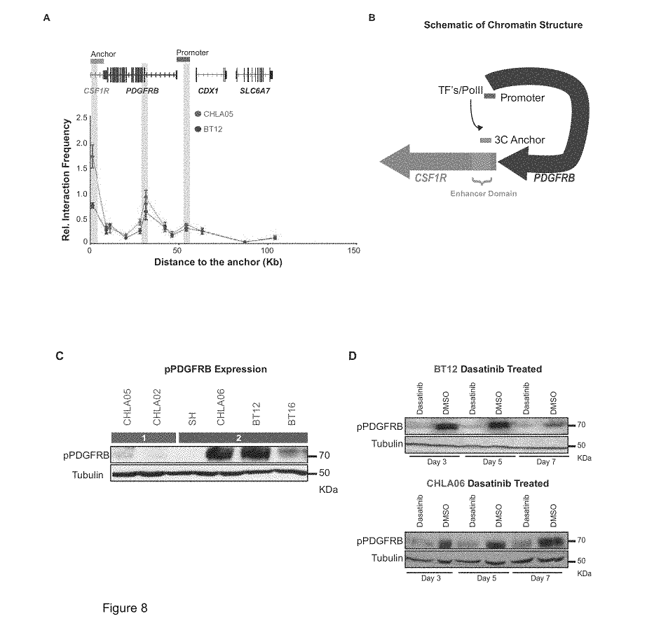

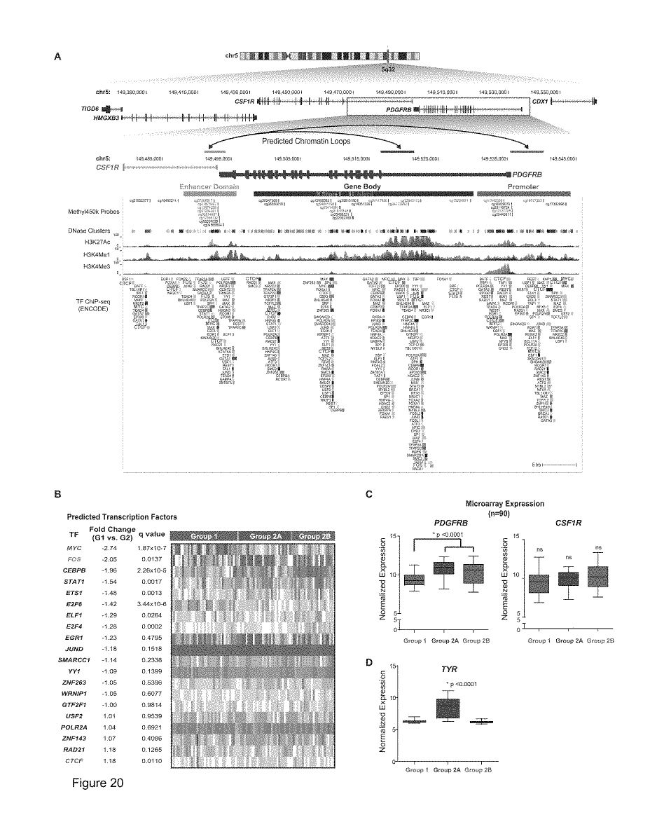

The integrated analyses of gene expression and methylation profiles in ATRT identified PDGFRβ as one of the most significantly differentially methylated and highly expressed locus in group 2A and B as compared to group 1 ATRTs. Importantly, CSF1R which maps tandem to PDGFRβ and encodes another potential Dasatinib/Nilotinib target, was not differentially methylated or expressed in primary group 1 and 2 ATRTs tumors or cell lines. These findings suggested differential epigenetic regulation of PDGFRβ may underlie the distinct sensitivity of group 2 ATRT cells to Dasatinib and Nilotinib. Consistent with high PDGFRβ expression in group 2 ATRTs, ATAC-seq analyses revealed an open chromatin pattern at the promoter region of PDGFRβ but not CSF1R, specifically in group 2 and not group 1 primary tumors and cell lines (FIG. 7A,B). Interestingly, ATAC-seq analyses also identified another distinct region of open chromatin in group 2 tumors and cell lines which corresponds to a potential regulatory domain located 50 kbs upstream of the PDGFRβ promoter within the first non-coding exon of CSF1R (chr5:149,491,285-149,493,716) (FIG. 7A,B). Detailed mapping of methylation patterns across the CSF1R and PDGFRβ transcriptional units (FIG. 7C) showed that the putative enhancer aligned with a region containing 6 CG residues that were hypomethylated in group 2 but not group 1 primary ATRTs and cell lines. Notably a comparison of methylation levels across the putative enhancer, the PDGFRβ promoter and associated CpG island and North shore regions as well as PDGFRβ gene body, revealed that hypomethylation at the putative enhancer element correlated most significantly with high PDGFRβ expression in group 2 primary tumors and cell lines. In contrast there was no significant correlation observed between methylation status of the putative enhancer and CSF1R expression (FIG. 7C). ENCODE data indicates this region exhibits differential H3K mono and trimethylation and H3K27 acetylation, as well as binding sites for multiple transcription factors including several Myc network proteins, FOS and CTCF (FIG. 7A; FIG. 20A) which are characteristic of enhancer elements (Filippova et al., 1996; Malik et al., 2014). Together with gene expression analyses which indicated significant enrichment of MYC, FOS and CTCF in group 2 ATRTs (FIG. 20B), these findings suggest differential epigenetic regulation of this putative enhancer element may underlie upregulation of PDGFRβ expression and distinct sensitivity of group 2 ATRTs to Dasatinib and Nilotinib. To confirm and map this putative PDGFRβ associated enhancer, H3K27ac ChIP-seq was performed on two Dasatinib/Nilotinib resistant group 1 (CHLA04, 05) cell lines and BT12, a representative Dasatinib/Nilotinib sensitive group 2 cell line. Peak analyses showed specific enrichment of H3K27 acetylation marks that aligned with the predicted enhancer region only in the group 2 BT12 cell line, and not in group 1 ATRT cell lines, thus suggesting the putative enhancer was only active in group 2 ATRT cells (FIG. 7B). To confirm functional and direct interaction of the predicted enhancer and the PDGFRβ promoter, Chromatin Conformation Capture (3C) quantitative PCR assays were performed which detects long-range physical interactions between genes and dispersed regulatory elements. 3C-qPCR assays were performed on group 2 cell line BT12 and group 1 cell line, CHLA05, using two probes, one anchored within the predicted enhancer element and the other within the PDGFRβ promoter region (FIG. 8A). These analyses revealed co-enrichment of probes mapping to the enhancer element and the PDGFRβ promoter region in BT12 and CHLA05 cells. Of note, although a second peak of enrichment mapping to the PDGFRβ gene body was observed, this did not correspond to a region of increased H3K27 acetylation in BT12 cells. Taken together with increased H3K27 acetylation marks at the putative PDGFRβ enhancer only in BT12 but not CHLA04, 05 cells, these data indicate chromatin looping facilitates direct interaction of a dispersed active enhancer and promoter to drive PDGFRβ expression in group 2 BT12 cells (FIG. 7B). Consistent with these observations, Western blot analyses showed high expression of phospho70 PDGFRβ in group 2 but not group 1 ATRT cell lines (FIG. 8C). Significantly, treatment of group 2 ATRT cell lines with Dasatinib robustly diminished expression of phospho-PDGFRβ (FIG. 8D). Collectively these results suggest that epigenetic regulation via differential methylation of a novel PDGFRβ associated enhancer element specifically drives sensitivity of group 2 ATRTs to small molecule inhibitors of the PDGFRβ signaling axis and identifies Dasatinib and Nilotinib as novel agents for the treatment of the particularly lethal group 2 ATRTs.

### Discussion

ATRTs are highly malignant cancers that exhibit substantial heterogeneity in patterns of disease presentation and poorly defined biology for which best therapeutic approaches remain to be defined. Here the present inventors demonstrate that ATRTs comprise 3 epigenetic subtypes which correlate with distinct tumor locations, patient age, lineage enriched methylation and transcriptional signatures, and unique global and SMARCB1 specific genotypes. Significantly, the data reveals ATRT sub-groups are associated with distinct epigenomic landscape and specific therapeutic vulnerabilities to small molecule inhibitors of NOTCH, BMP, PDGFRβ and epigenetic signaling. Furthermore, differential methylation of a novel PDGFRβ associated enhancer underlies the robust and distinct sensitivity of group 2 ATRTs to Dasatinib and Nilotinib, two well characterized and widely used cancer drugs.

Studies of human tumors increasingly indicate that convergence of epigenomic features, which often reflect tumor cell of origin and specific genetic alterations underlie diverse tumor phenotypes (Feinberg et al., 2006). Indeed ATRT sub-types with specific lineage enriched methylation signatures were associated with distinct tumor location and age of presentation suggestive of origins from different neural progenitor compartments. Specifically distinct methylation and expression patterns of neurogenic loci including forebrain markers LHX2 (Roy et al., 2013) and MEIS2 (Cecconi et al., 1997) as well as FABP7 (Sharifi et al., 2011) and ASCL1 (Nelson et al., 2009) which are markers of radial glial neural progenitor cells (Anthony et al., 2004) were observed in the predominantly supra-tentorial group 1 ATRTs, and suggest these as potential cell of origins for group 1 ATRTs. In contrast, differentially methylated and expressed loci in group 2 ATRTs primarily had functions in mesenchymal lineage/signaling (BMP/PDGFRβ) and hindbrain development (ZIC1,2,4,5,OTX2, HOXB/C) indicating group 2A and B ATRTs which comprise primarily infratentorial and spinal tumors may develop from mid/hindbrain neural progenitors. Enrichment of neuronal development pathways in group 1 ATRTs contrasted with a dominance of stem cell differentiation and pluripotency pathways in group 2A ATRTs. Notably in contrast to group 1 and 2B ATRTs, group 2A tumors were associated with global CpG island hypomethylation, a more open chromatin landscape and overall increase gene expression patterns reminiscent of more primitive cell types. These data further suggest group 2A ATRTs which arise in the youngest patients (12.00 months 95% CI 11.05-13.00) may originate from highly primitive neural precursors.

Remarkably, although coding mutations were rare across all ATRTs, molecular sub-groups of ATRTs had distinct patterns of SCNAs and SMARCB1 genotypes thus suggesting that ATRT sub-types may be associated with different mechanisms of tumor initiation and progression. Of note broad SMARCB1 genomic deletions in group 2B tumors frequently encompassed other candidate chr 22 modifier loci, including BCR, MKL, EP300, with functions in neural development and chromatin remodelling (Kaartinen et al., 2001). An intriguing observation was a trend towards enrichment of SMARCB1 point mutations predicted to result in a partially truncated protein product in group 1 ATRTs. Although ATRTs have long been considered to result from abolition of SWI/SNF function associated with SMARCB1 loss, there is increasing evidence that residual SWI/SNF activity may drive tumorigenesis (Wilson et al., 2014). Together with methylation and transcriptional data which suggest different cellular origins of ATRT subtypes and the known diverse context/lineage dependent composition of SWI/SNF complexes (Kadoch and Crabtree, 2015), molecular sub-types of ATRTs may also reflect cell context dependent effects of specific SMARCB1 alterations and consequent distinct epigenetic activities of residual SWI/SNF complexes.

Based on the lack of other recurrent genetic alterations in ATRTs, there has been substantial interest in targeting epigenetic mechanisms in ATRTs. Specifically, promising in vitro and in vivo studies using EZH2 (Knutson et al., 2013) and BET domain (Tang et al., 2014) inhibitors have been reported. Intriguingly, while the present screen of small epigenetic inhibitors has confirmed the therapeutic effects of UNC1999 and JQ1, respectively EZH2 and BET domain inhibitors, growth inhibitory effects were observed only in group 1 ATRT cell lines. Similarly, only group 1 ATRT cell lines were sensitive to UNC0638, a chemical probe for histone methyl transferase G9a, while LAQ824, a histone acetylase inhibitor had no sub-type specific effects. These findings are consistent with more general epigenetic functions of histone deacetylases as compared to histone methyl transferases. Interestingly, these patterns of sensitivity overlapped with inhibitors of NOTCH and BMP signaling which have critical functions in lineage specific progenitor cell survival (Ericson et al., 1998). Specifically, group 1 ATRT cells with neurogenic transcriptional and epigenomic profiles were sensitive to DAPT, UNC0638 and UNC1999 while group 2 cell lines which exhibit very limited features of neural differentiation, were largely insensitive to all 3 inhibitors. In contrast distinct sensitivity of group 2 ATRT cell lines was observed to inhibitors of BMP and PDGFR6, both mediators of mesenchymal signaling. Of note, recent reports indicate functional and physical interaction of the G9a/GLP and Polycomb Repressive Complex 2 (PRC2) epigenetic silencing machineries and co-regulation of neuronal developmental genes by G9a and PRC2 (Mozzetta et al., 2014). Taken together, these observations indicate that lineage associated epigenomic landscapes of ATRT have implications for development of ATRT sub-type specific therapeutics.

Multi-tyrosine kinase inhibitors, Dasatinib and Nilotinib, were identified as potential new drugs for group 2 ATRTs. These findings have significant immediate clinical implications as the safety and efficacy of both agents as anti-cancer drugs have been established in adults and children. Importantly, both drugs have demonstrated blood brain permeability which is often a critical limitation of translating drug discoveries for brain tumors into clinical practice. Interestingly, in keeping with observations that transcriptomes of extra CNS rhabdoids are also enriched for BMP signaling/mesenchymal lineage genes (Birks et al., 2011; Gadd et al., 2010), methylation profiles of extra-CNS rhabdoid tumors overlap with that of group 2 ATRTs. These observations suggest that common or closely related cellular origins may underlie the development of a subset of group 2 ATRTs and non-CNS tumors which is characteristically seen in very young children with rhabdoid predisposition syndrome. Indeed, both drugs also robustly inhibit the growth of G401, a cell line derived from a renal rhabdoid tumor (data not shown). Thus Dasatinib and Nilotinib may also represent novel therapies for non-CNS rhabdoid tumors.

Despite evidence of a critical etiologic role for SMARCB1 in rhabdoid tumor initiation, the pathobiology of ATRTs remain poorly elucidated. The collective data herein suggest that SMARCB1 loss via diverse mechanisms in different cellular context together with additional epigenetic and genetic events underlie the heterogenous clinical phenotypes seen in human ATRTs. These observations have important implications for fundamental understanding and targeting of SWI/SNF function in neoplastic cell growth as well as significant immediate clinical implications for the management of ATRTs. Specifically, the analyses which reveal a spectrum of novel alterations dispersed throughout SMARCB1 indicate that current diagnostic methods may underestimate the frequency of constitutional and tumor related alterations in ATRTs. The present inventors have demonstrated and identified both known and potential novel drugs and drug like inhibitors with remarkably different therapeutic effects in molecular sub-types of ATRTs. Most importantly the present study underscores the significant limitations of current chemoradiotherapeutic regimens uniformly used for all ATRTs patients.

### Experimental Procedures

### Tumor and Patient Information

All tumor material and clinical information were collected through an international collaborative network for study of ATRTs. In total, 194 CNS (191 primary and 3 recurrent) and 9 non-CNS rhabdoid samples were collected for genomic analyses. All ATRTs were diagnosed according to World Health Organization (WHO) CNS tumor classification criteria (Louis and Wiestler, 2007) and confirmed by BAF47 immuno-negativity (BD Biosciences, USA, Cat #612110). Biallelic SMARCB1 alterations were confirmed using FISH, MLPA, Sanger sequencing of SMARCB1 exons 1-9 exons or next generation sequencing. Tumor samples and clinical information were collected with consent as per protocols approved by the Hospital Research Ethics Board at participating institutions. DNA or RNA from snap frozen tumor were investigated with one or more of whole genome, exome and RNA sequencing and high resolution copy number/SNP, gene expression and methylation array analyses; 123 samples with DNA from FFPE materials were analysed with methylation array (QIAmp DNA FFPE Tissue Kit Cat #56404).

### Next Generation DNA and RNA Sequencing

Whole genome/exome and RNA sequencing were performed respectively at Genome Quebec Innovation Centre, Montreal, Quebec and at The Centre for Applied Genomics, Toronto, Ontario. For DNA library preparation, 2-3 μg of high molecular weight genomic DNA (gDNA) was fragmented using a Covaris E210 and prepared using the TruSeq DNA Sample Prep Kit (v1 FC-121-1001, FC-121-1002, Illumina Inc, San Diego, Calif.) as per the manufacturer's specifications with modification for size selection of fragments (between 450-550 bp for whole genome shotgun or 350-4500 bp for whole exome) performed on a 1.5% gel Pippin Prep cassette (Sage Science, Beverly, Mass.). For exome preparation the standard manufacturer procedures were followed using the IlluminaTruSeq exome enrichment kit. RNA preparation was performed using the IlluminaTruSeq RNA sample preparation kit for poly-adenylated mRNA selection, or using Illumina RiboZero Stranded library preparation kit for total RNA sequencing.

### WES/WGS Sequence Alignment and Variant Calling

A total of 69.95 billion reads were generated for whole genome sequencing of 23 matched normal lymphocyte/tumor samples, respectively (average of 3.04 billion reads per tumor/normal pair) using the IlluminaHiSeq 2000 platform with a mean sequencing coverage of 47.76× and 37.66× respectively for tumor DNA and normal lymphocyte. Fifteen tumor normal pairs were sequenced using paired-end 100 bp length reads, and 8 pairs were sequenced using 125 bp reads. For whole exome sequencing, a total of 66.02 billion reads were generated for 27 samples, 5 had matched normal lymphocyte DNA, and were sequenced to a mean CODS coverage of 66.86. Illumina sequence adapters were removed and reads were trimmed from the 3′ end to have a phred score of 30 using Fastx software (v0.0.13.1). Reads were aligned to hg19 (NCBI build 37, August, 2012) reference genome using BWA aligner (v0.6.2). Local re-alignments were performed using GATK (v2.1-9g6149b06) to reduce false positive rates across putative indels where mismatch bases are preferred over indel calls during alignment, and 5′ PCR-duplicated reads were marked using Picard software. Somatic SNV and small indel calling was performed using SAMtools mpileup and bcftools varfilter (v0.1.18) with default parameters and the bcftools—T pair option enabled to compute a phred-scaled likelihood ratio (CLR score) to score somatic variants. Low-quality variants were filtered using a minimum depth coverage of 2, maximum coverage of 1200 and a minimum RMS mapping quality of 15.

Detection of Somatic Structural Variation (SV) from Next Generation Sequencing Data

Four orthogonal SV detection algorithms were employed—CREST (Wang et al., 2011), BreakDancer (Chen et al., 2009), and Pindel (Ye et al., 2009), and the trans-ABySS pipeline (Robertson et al., 2010) on whole-genome sequencing data from n=23 matched tumor/lymphocyte DNA. CREST was run with default parameters using germline subtraction of soft-clipped reads option to select for somatic variants. Dual-sided soft-clipped reads with one side having at least 3 supporting reads were required and SV's affecting genetic loci defined in RefSeq (hg19, Build 37, August 19, 2012) were retained. All inversions, translocations and large insertions/deletions were visualized at the alignment level using IGV and examined by a post-filtering assembly process. CREST was also run on WES samples (n=27 tumors, 5 with matched lymphocyte DNA) and filtered as previously. Discordant reads were extracted from the BAM files and assembled using the Velvet algorithm (Zerbino and Birney, 2008) within Geneious Software Suite (v5.6.5). Assemblies provided a contig spanning the predicted breakpoints of structural events which were re-aligned to the reference sequence using BLAT. A structural event was considered pre-validated if the contig unambiguously mapped to the two breakpoint regions. Events were compared to the Database for Genomic Variants (v10) to exclude normal population SVs. Orthogonal tools BreakDancer and Pindel were used in parallel. BreakDancer was run with the following parameters: (1) minimum mapping quality 35, (2) read-pairs within +/−3 standard deviations of insert size for tumor and +/−2 standard deviations for normal were excluded. Pindel was run according to the following parameters: (1) minimum match around breakpoint 10 bp, (2) minimum match to reference 50 bp, (3) minimum read mismatch rate 0.1. For both tools, somatic events were selected by requiring read call support of ≧10 reads in the tumor with 0 supporting reads in the normal. Hyper- and hypo-mappability regions and microsatellite regions were also discarded. Finally, the trans-AbySS pipeline (v1.4.8) was performed according to previous publications (Cancer Genome Atlas Research, 2013) (Morin et al., 2013). Briefly, de novo assembly of sequencing reads across multiple k-mer values was performed and merged into a non-redundant set of contigs. Assembled contigs were aligned using BLAT to human reference genome hg19, and discordant alignments not matching known gene annotations (RefSeq, UCSC) were identified as putative gene re-arrangements. Further filtering of candidates was conducted by mapping reads back to contigs using Bowtie (v1.0.1), and to the genome using BWA (v0.7.8), and putative fusions were maintained by requiring at least 2 reads spanning contig breakpoints and at least 4 flanking paired-end supporting reads. Somatic events were selected by comparison of tumor calls to normal lymphocyte DNA. Detection of CNVs from WGS data was performed using a Hidden Markov Model (HMM) approach, which compares tumor to normal lymphocyte using HMMcopy (v0.0.6) (Ha et al., 2012). APOLLOH (v0.1.1) (Ha et al., 2012) was also employed, which compares high confidence SNP allelic ratios between tumor and normal DNA for detection of loss of heterozygosity. All putative structural events were then filtered using the database for genomic variation (July 2013) to exclude normal variants in the healthy population, and then subjected to visual inspection of alignments using the Integrated Genomics Viewer (IGV).

### Alignment and Processing of RNA-Sequencing Data

An average of 97.64 million reads/sample was generated and reads were aligned using the Tophat algorithm (n=5 tumors, n=1 normal). Adapter sequences were trimmed using Trim Galore (v0.2.5) and aligned using TopHat (v2.0.5), with an average of 92.13 million reads aligned per sample. The hg19 reference genome and the UCSC gene annotation file were used for alignment to known genes. Tophat was run with the fusion search function enabled for detection of potential fusions using the following parameters: segment length=50, mate-std-dev=80, fusion-anchor-length=13, fusion-multipairs=4, fusion-min-dist=100000. Candidate fusions were manually curated to include only fusions with at least 10 spanning reads or 5 read pairs and annotated according to hg19 RefFlat (Aug. 19, 2012) using custom scripts based on BEDtools. For samples prepared using the RiboZero protocol, the fr-firststrand library-type option was enabled to allow for strand-specific alignments. Putative events were inspected at the alignment level using IGV and further corroborated by extraction of discordant reads, Velvet assembly and re-alignment using BLAT. Gene expression values were represented as FPKM values generated from RNAseq alignments using cuffquant and cuffnorm programs within the Cufflinks RNAseq assembly suit (v2.2.1) (Trapnell et al., 2012) with geometic library normalization and blind replicate dispersion estimation.

### ATACseq Sample Preparation and Bioinformatics Analysis

Snap-frozen ATRT primary tissue or freshly cultured cell lines were prepared for ATACseq according to previously published methods with minor modifications (Buenrostro et al., 2013). Briefly, nuclei were prepared from ˜50,000 cells by spinning at 600×g for 10 minutes, followed by a wash using 50 ul PBS buffer, and further centrifugation at 600×g for 5 minutes. Cells were lysed using cold lysis buffer (10 mM Tris-HCl, pH 7.4, 10 mM NaCl, 3 mM MgCl₂, 0.1%), and subsequently centrifuged for 10 minutes at 600×g at 4° C. The supernatant was removed and pellet resuspended in 50 ul of transposase mix (25 ul of 2×TD Buffer, 2.5 ul of transposase [TD enzyme; Illumine], 22.5 ul of water) for 30 minutes at 37° C. Next, library amplification was performed using the NEBnext High Fidelity 2×PCR Master Mix (NEB #M0541S) according to previously published PCR conditions (Buenrostro et al., 2013). PCR reactions were purified using QIAGEN miniElute kit, and a following size selection step was performed using LabChip (#760601). ATACseq library preparations were sequenced using single-end 50 bp reads on the Illumina HiSeq 2000 platform. Raw reads were adapter-trimmed using Trim Galore (v0.2.5) and aligned to the genome with Bowtie (v1.0.1) with the m1 option enabled to allow for only uniquely aligned high-quality reads. Peaks were called using the MACS2 software (v2.1.0.20140616) (Zhang et al., 2008) with the options—q 0.05 to retain significant peaks, —shiftsize 50 to account for the transposase fingerprint, and otherwise default parameters were used. Tag count libraries and bedgraph files were constructed using HOMER software (v4.7) (Heinz et al., 2010). Correlation and PCA analyses was performed using the DiffBind software (v1.16.2) (Ross-Innes et al., 2012).

### H3K27ac ChlPseq Sample Preparation and Bioinformatics Analysis

Formaldehyde cross-linked chromatin from 5×10⁶ CHLA04, CHLA05, and BT12 cells were fragmented via water bath sonication (20-40 cycles, max amplitude, 30 second intervals) to an approximate fragment length of 200-400 bp. After centrifugation of fragmented chromatin, the supernatant was incubated with H3K27ac antibody (Abcam Cat #ab4729) (preincubated for 6 hours with Dynabeads A and G [Invitrogen Cat #10002D, 10004D]) overnight at 4° C. Antibody-free input was retained as a control. After incubation, chromatin was decrosslinked using decrosslinking buffer (1% SDS and 0.1M NaHCO₃) and DNA was purified using QIAGEN QIAquick PCR purification kit (Cat #28104), and further quantified using PICO green. Sequencing was performed similarly as ATACseq using single-end 50 bp reads on the Illumina HiSeq 2000 platform. Alignment and peak calling was performed as above with the exception of removing the -shiftsize=50 parameter used for ATACseq.

### Chromatin Conformation Capture Analysis

Chromatin conformation capture (3C) was performed on BT16 and CHLO5 cells. The 3C library preparation followed two previously published reports (Hagege et al., 2007) (Zhang et al., 2012) with modifications suggested by Court et al 2011 (Court et al., 2011). Briefly, ˜8 million cells were prepared for chromatin cross-linking with 1% formaldehyde for 10 min at room temperature with rocking. The reaction was quenched with glycine. Following washing and incubation with permeabilization buffer, the nuclei of the cells were digested with Hind III by adding serially 150 units (U) of restriction enzyme for a total of 450 U. 150U of Hind III-HF was added and incubated at 37° C. for 2 h shaking at 900 rpm, then added gently for a second time more 150 U of Hind III and incubated at 37° C. for 2 h shaking at 900 rpm, then added again 150 U of HindIII for an overnight digestion at 37° C. shaking at 900 rpm. To inactivate restriction enzyme, SDS (final 1.6%) was added and incubated for 30 min at 37° C. The cross-linked and digested DNA was ligated at low DNA concentration (T4 ligase 4000 units, 4 h at 16 C and then 30 min at room temperature). Cross-linking was reversed by incubation at 65° C. for h in the presence of Proteinase K (40 μg/ml) followed by phenol/chloroform/ethanol DNA clean up.

3C interaction products were detected by qPCR using SYBR green with candidate primer pairs (anchor and bait/controls) following an adapted previously published report. The reaction for detecting 3C interaction was performed using KAPA Sybr Fast qPCR Master Mix, 175 nM of the anchor forward primer and bait/controls reverse primers (final concentration) and 20 ng of 3C libraries (adjusted after quantification). Samples were tested in triplicate for amplification detection. The PCR conditions were 95° C. for 3 min followed by 40 cycles of 5 s at 95° C., annealing and extension 30 s at 64° C. The qPCR results from 3C-processed sample were normalized to serial dilutions (standard curve) of 3C-positive control template on each plate. The positive control was generated by synthesis of all possible PCR products using the available primers, followed by gel extraction and purification. PCR products were mixed in equimolar concentrations, digested with Hind III and purified by phenol/chloroform extraction and ethanol-acetate precipitation. The digested fragments underwent random ligation (T4 ligase) at high DNA concentration and purified with MiniElute PCR. To mimic 3C sample condition, the concentration of control template was adjusted by addition of genomic DNA that had undergone digestion and random ligation to the control template, increasing the complexity of the control. This way the PCR efficiency was not affected by the total amount of the DNA present (only target regions in the control template). A published normalization method was used for data analysis (Hagege et al., 2007). The final value was calculated using value=10(Ct−b)/a (b: intercept and a: slope). These values were normalized to an internal control (GAPDH).

TABLE 1
Primers used for 3C analyses
Primers for Chromatin Confirmation Capture

| | | | SEQ ID |
|---|---|---|---|
| Fragment | Forward Primer (5′-3′) | Reverse Primer (5′-3′) | ID: |
| Anchor | TCTGGGCAGTGACAAAACCATACC | AGACCACGGGACCTCTTTCACT | 1,2 |
| FR1 | CCCTGCAGTTTTCTTGCCTCCTA | CTACCCTGCCCTGCCTGAAG | 3,4 |
| FR2 | CCCACCACAAAGCACTGTCATG | TGATCGTTGTAAACAGTGGCCTTT | 5,6 |
| FR3 | GTTGGAACCACAGGACTGGAAT | ATGGAGAACCTGTGATTCTACTGAA | 7,8 |
| FR4 | GGGGAAGCAGGCTCAGAGAGAT | GCAGAGAGAGGATGGAGCTTGT | 9,10 |
| FRS | GGACAGACAGGACAGTGCAAGA | GCTCAGAGAGGGTCAGGACTGT | 11,12 |
| Bait/ | AGTCCTCAGAACAATCCCATGACA | GGAGCCTGTCTGCCCAGTATTA | 13,14 |
| promoter | | | |
| FR6 | CTGGGTGGATGGGAGTTCTTGT | CCACTGACCACCTCTCCAATCT (w/R) | 15,16 |
| FR7 | CTGGGTGGATGGGAGTTCTTGT (w/F) | CCACTGACCACCTCTCCAATCT | 17,18 |
| FR8 | AAGGGAGATTATGCAGTGGTTTGT | TGGAACACAGGAGCAGGAAACA | 19,20 |
| FR9 | TGCCAGGACAGAGAGGAGTAATT | AAACTCCCGTCCCCTAATGCAT | 21,22 |
| FR10 | CACAGGGCATGGTAGACGTACT | CCCAGCCCTGCCTTCACTTG (w/R) | 23,24 |
| FR11 | CACAGGGCATGGTAGACGTACT (w/F) | CCCAGCCCTGCCTTCACTTG | 25,26 |
| GAPDH | CCACTCCTCCACCTTTGAC | ACCCTGTTGCTGTAGCCA | 27,28 |

### Copy Number Analysis

OmniQuad SNP array analyses were performed on 60 primary and 2 recurrent ATRTs. Probe fluorescence intensity normalization was performed using Illumina's GenomeStudio (v. 2011.1, Genotyping Module 1.9.0) and represented as Log R ratio (Log₂ [Rₑₓₚᵣᵢₘₑₙₜ/R_{controlset}]) and B Allele Frequency (BAF) plots. SNP positions were based on the hg19 (Build 37, August, 2012) human reference genome. SNP data was analyzed using orthogonal tools: DNAcopy, dChip, Partek, and ASCAT (Allele Specific Copy Number Analysis of Tumors). Normalized Log R ratios were imported into the R statistical language environment (v2.14) and analysed using the Circular Binary Segmentation (CBS) algorithm provided in the DNAcopy R package (v1.32.0). Segments with <10 markers were filtered out, and copy number regions were classified according to the following criteria: homozygous loss: R ≦−0.5, heterozygous loss: −0.5<R<−0.1, balanced (2n): |R|0.1. gain: 0.1<R<0.2, amplification: R≧0.2. Partek and dChip were also used to detect copy number variants. For Partek, genomic segmentation was performed using a 150-probe bin size and events with <10 markers were discarded. For dChip, Log R Ratios were exported from Genome Studio and normalized using the MBEI algorithm. Available matched normal lymphocyte DNA samples from 11 ATRTs were used as a diploid reference, and resulting copy number estimates were compared against the human HapMap project and the Database of Genomic Variants to exclude normal population CNVs. Focal CNVs (less than 12 Mb) were annotated using the HG19 RefFlat with custom scripts to identify candidate targets that mapped within regions of CNV. Tumor purity and ploidy was assessed using ASCAT with default parameters.

### Determination of Somatic Mutation Rates Across the ATRT Genome

To define genomic somatic mutation rates, a subset of 881 somatic mutations (CLR ≧35) were visually validated to establish the sensitivity and specificity of CLR score as a predictor of somatic status (CLR scores from 35-200 were tested). For each CLR score tested, sensitivity and specificity were calculated using the following formula:

Specificity=TN/(TN+FP)and Sensitivity=TP/(TP+FN)
TN=True negative; Non-somatic mutation with CLRₘᵤₜₐₜᵢₒₙ<CLR_{tested}
FP=False positive; Non-somatic mutation with CLRₘᵤₜₐₜᵢₒₙ≧CLR_{tested}
TP=True positive; Somatic mutation with CLRₘᵤₜₐₜᵢₒₙ≧CLR_{tested}
FN=False negative; Somatic mutation with CLRₘᵤₜₐₜᵢₒₙ<CLR_{tested}

It was determined CLR=68 provided a sensitivity and specificity of 89% for determining true somatic status of a mutation, and this score was used to calculate the mutation rate for the 13 primary as well as 2 recurrent ATRTs with matched lymphocyte DNA in both the CODS coding regions and in the whole genome. Chromosomes 1-22, X and Y and mutations with an overall minumum depth of coverage of at least 10× were included. Mutations were categorized into single nucleotide variations (SNVs) and small insertions/deletions (indels), and mutation rate was determined as the total number of mutations/total megabases covered (callable region) for the coding region and for the genome. Callable region was defined as the percentage of CODS or genome coverage at a depth of 10x multiplied by the total length of the genome or of the CODS coding region. Mutations with CLR ≧68 were also used to categorize nucleotide transitions and transversion profiles using custom scripts written in python.

### Gene Expression and Methylation Array Data Processing

For gene expression analysis (90 primary and 2 recurrent ATRTs), probes were collapsed into genes by taking the average value, quantile normalized using the Lumi R package (v. 2.11) and batch corrected using ComBat (Broad Institute)(Johnson et al., 2007). For methylation 450 k analyses, only probes with a detection p value less than 0.01 and bead count >3 were retained for analysis. Data (162 primary and 2 recurrent ATRTs) was normalized using BMIQ algorithm to obtain beta values for downstream analyses using the ChAMP package (v1.4.1) (Morris et al., 2014). All X and Y chromosomes probes (n=11,649), single-nucleotide polymorphisms (dbSNP, n=88,679), and unannotated probes (relative to HG19, n=65) were excluded leaving a total of 385,184 probes for methylation analyses.

### Gene Expression and DNA Methylation Analyses to Define ATRT Sub-Groups

Two orthogonal unsupervised consensus cluster methods were applied to define the number of ATRT molecular sub-groups using gene expression and methylation data. Gene expression and methylation data were independently examined using consensus unsupervised hierarchical clustering (Consensus Cluster Plus) (v1.20.0) (Wilkerson and Hayes, 2010) and Non-negative Matrix Factorization (NMF; Broad Institute) consensus cluster analyses. For these analyses, genes and methylation probes were ranked based on co-efficient of variation for gene expression or standard deviation for methylation data. For consensus HCL analyses, re-iterative analyses of gene expression and methylation data sets using 200-2,000 genes or 6,000-30,000 methylation probes were performed to reveal the optimal number of molecular classes over a range of 2-10 k classes. Consensus HCL analyses of gene expression and methylation data revealed ATRTs comprised three molecular sub-groups. Next, to determine the optimal, most stable sub-group classification, NMF analyses were performed using the same range of probe sets with k set to 3, to determine the number of probes with the highest cophenetic coefficient. In addition to strong agreement between orthogonal techniques, the concordance between gene expression and methylation tumor grouping was also evaluated using the Rand index to determine cluster agreement. Methylation and gene based clusters were most stable using probe sets of 10,000 and 250 probes or genes, respectively, and were used to assign tumor subgroup for downstream analyses. Silhouette analyses were performed using the optimal probe and gene sets to identify outlier samples. Samples with a negative silhouette width were excluded from further statistical analyses.

### Pathway Analyses of Gene Expression Data

To define molecular and cellular features of the three ATRT subgroups, parallel and integrated analyses of gene expression and methylation data were conducted. Genes and probes enriched within tumor sub-groups were respectively determined using a supervised t-test or Wilcox-Rank Sum test adjusted for multiple hypotheses testing using the Benjamini-Hochberg FDR method (q≦0.05) to identify genes and probes with respective +/−2 fold or +/−0.1 fold changes in expression or methylation levels between tumor sub-groups. Magnitude of gene expression (log₂) or methylation difference (beta values) and significance of change (−Log₁₀ q value) were visualized using volcano plots with a significance threshold of 1.3−Log₁₀ q value (q=0.05) of one subgroup relative to the other two subgroups. To define enriched biological processes associated with tumor sub-groups, Ingenuity Pathway Analysis (IPA) (http://www.ingenuity.com/) was performed using gene expression data +/−2 fold with an FDR q value less than 0.05.

### Validation of Genomic Alterations

For validation of SNVs and indels from next generation sequencing data, PCR primers were designed in-house and applied to the Fluidigm Dynamic 48×48 array (http://www.fluidigm.com/). Primer pairs were multiplexed and PCR was performed according to manufacturer's instructions. PCR products were pooled and sequenced using lonTorrent or Illumina MiSEQ sequencing platform according to the manufacturer's instructions. For structural variants, individual primers were designed in-house and Sanger sequencing was performed with Big Dye Terminator v3.1 according to the manufacturer's procedures (Applied Biosystems). PCR was performed using KAPA2G Fast Hotstart (Kapa Bioscience).

### Cell Culture Viability and Migration Assays

ATRT cell line CHLA02 (ATCC, Cat #CRL-3020), CHLA04 (ATCC, Cat #CRL-3036) were cultured according to the manufacturer's protocol. CHLA05 and CHLA06 cells, which were derived from primary ATRTs by Dr. Anat Erdreich-Epstein from the Children's Hospital of Los Angeles, were cultured in DMEM/F12 supplemented with 20 ng/mL FGF, 20 ng/mL EGF, and 1× B27 supplement. SH cells were obtained from Dr. Shih Hwa Choiu at Taipei Veterans General Hospital and was cultured as previously described (Kao et al., 2005). BT12 and BT16, which were kind gifts from Dr. Peter Houghton at Nationalwide Children's Hospital, were cultured in RPMI-1640 supplemented with 10% FBS. ATRT 78C cells was established from a corresponding primary ATRT (T13; diagnosed at The Hospital for Sick Children) and cultured in Neurobasal medium supplemented with 75 ng/mL BSA, 10 ng/mL FGF1, 10 ng/mL EGF, 2 ug/mL Heparin, 1× B27 and 1× N2 supplements. CHLA266 was grown in IMEM media with 10% FBS and 1× ITS. G401 (ATCC, Cat #CRL-1441) was grown in McCoy's 5A media with 10% FBS.

To assess effects of NOTCH and BMP signaling on ATRT cell phenotypes, cells were treated with N-[N-(3,5-Difluorophenacetyl)-L-alanyl]-S-phenylglycine t-butyl ester (DAPT; EMD Chemicals, Germany, Cat #565770) or Dorsomorphin (DM; Sigma-Aldrich, USA, Cat #P5499) and analyzed using MTS assays for cell viability. Similarly, Dasatinib (Selleckchem, US. Cat #S1021 and Nilotinib (Selleckchem, US. Cat #51033) were used to assess PDFG/Src signaling. Cells were seeded at a density of 30% confluency per well and treated either with DMSO vehicle control or varying doses of DAPT, DM, Dasatinib or Nilotinib. On specific days (days 1-5), cells were evaluated with the CellTiter 96 Cell proliferation assay kit (Promega, USA, Cat #G3580) as per the manufacturers instructions. Epigenetic drugs/probes including Bromodomain proteins (JQ1, PFI-1, 2, 3, GSK2801, SGC-CBP30), Methyltransferases (GSK343, UNC1999, UNC0642, UNC0638, A-366, LLY507) and histone deacetylases (LAQ824) were obtained from the Structure Genomics Consortium (http://www.thesgc.org). For epigenomic drug screening experiments, conditions were set up identically with the exception of absorbance readings being taken at 3 and 7 days post-treatment. Cell viability was determined based on mean cell absorbance at 492 nm.

### Western Blotting and Immuno-Histochemical Analyses

Nuclear and whole cell lysates were prepared using cytosol lysis buffer or nuclear buffer F (Sommer et al., 1998) respectively and immunoblotted with antibodies to SMARCB1/BAF47 (BD Biosciences, USA, Cat #612110), cleaved NOTCH1 (Cell Signaling, USA, Cat #2421S), pSMAD1/5 (Cell Signaling, USA, Cat #95165), SMAD1 (Cell Signaling, USA, Cat #9743S) or a-Tubulin (Sigma-Aldrich, USA, Cat #T9026) as per standard methods. Phospho-PDGFRβ P70 was obtained from Santa Cruz Biotechnology (Santa Cruz, Calif. USA, Cat #sc-12909-R). BAF47 immuno-staining of tumor samples was performed by the Hospital for Sick Children Pathology Department (Toronto, ON, Canada) as per standard clinical protocols.

### TUNEL and Transwell Migration Assay

TUNEL assays were performed using in situ cell death detection kit, Fluorescein, according to manufacturer's instructions (Roche, West Sussex, UK). Pictures were taken at 20× magnification. Percentage of TUNEL positive population was calculated with respect to DAPI-positive cells, in five random fields per cell cultured slide, and averaged as representative population. Fold-changes were obtained by comparing with untreated control. Cell migration and invasive phenotypes of ATRT cell lines was assessed using a Transwell Boyden chamber assay according to manufacturer's instructions (BD Science). In brief, 3.5×10³ cells were seeded onto membranes with 8 μm pores in Boyden chambers, and incubated at 37° C. Membranes were removed after 18 hours and examined at 10× magnification under the microscope. To enumerate the number of cells that had invaded and migrated, cell counts from five random fields on the membrane was used to estimate the total number of cells migrated across the transwell membrane.

qRT-PCR Analyses

cDNAs were synthesized from high quality RNA using the High Capacity cDNA Reverse Transcription Kit (Applied Biosystem, Cat #4368814) as per the manufacturer's instructions and qRT-PCR analyses were performed using purchased Taqman probes and the TaqMan® Gene Expression Master Mix (Invitrogen) (BAMBI, SOST, BCR [exon2, 8, 16, 18, 21, 23], RGL4, CHCHD10, MMP11, SMARCB1 [exon 2, 5]). A subset of analyses was performed with Platinum SYBR Green qPCR reagent kit (Invitrogen) as per the manufacturer's protocol with in-house designed primers to amplify invariant exons (HES1, HES5). For gene expression analysis of DAPT or DM treated cells, RNA was isolated from test and control cell lines at four days post-treatment. Synthesized cDNA were analysed with gene specific probes using the Platinum SYBR green qPCR reagent kit. The ΔCₜ or ΔΔCₜ method was used to calculate mRNA expression levels.

TABLE 2
Primers used for qRT-PCR analyses
qRT-PCR

| | | | SEQ ID |
|---|---|---|---|
| Gene | Forward Primer (5′-3′) | Reverse Primer (5′-3′) | ID: |
| BAMBI | GGGCAGGTTGCAAAGTTAGA | ATCGTTGCTGAGGTCTGCTT | 29,30 |
| (Ex3) | | | |
| BCR (Ex2) | TCGGAACACCACCTGGATAC | GATGAGGAGGGCGAGTCAT | 31,32 |
| BCR (Ex8) | CTTGCTGAAGCACACTCCTG | GGGTGTGATCTCCTCATTGAT | 33,34 |
| BCR (Ex16) | GCTCCCAGACCCTGAGGATAC | CCCTTCCCCATGAGTCTGTC | 35,36 |
| BCR (Ex18) | AGCTCTCGGTCAAGTTCAACA | TTGGTGACCACAGCAATCTT | 37,38 |
| BCR (Ex21) | TCTTTCAGACCCGGTTGC | TTTTCAGGTGGTCCAGAAGG | 39,40 |
| BCR (Ex23) | TGATGATGAGCGAGATGGAC | CTGCGAAGTTGGGGTAGAAC | 41,42 |
| CHCHD10 | GCCTACGAGATCAGGCAGTT | GGTAGTACTTGCACTGCTTCAGG | 43,44 |
| (Ex3) | | | |
| HES1 (Ex4) | GAGAGGCGGCTAAGGTGTTT | GTGTAGACGGGGATGACAGG | 45,46 |
| HESS (Ex3) | CTTTTGTGAAGGCCGAACTC | CACACTCAGGAGCCTTTTGG | 47,48 |
| MAIP11 | ATGCAGCCCTGCCCAGTA | GGCACTCAGCCCATCAGA | 49,50 |
| (Ex2) | | | |
| RGL4 | CTGGCTGGTCTTTCTCCTTG | AACCTTTGGAGAACCCCAGT | 51,52 |
| (Ex11) | | | |
| SM4RCB1 | TCCGTATGTTCCGAGGTTCT | TTTACCATGTGACGATGCAA | 53,54 |
| (Ex2) | | | |
| SM4RCB1 | CCCAGCTGTGATCCATGAG | TCCAGGTGAAGGCGTCTC | 55,56 |
| (Ex5) | | | |
| SOST (Ex2) | ATGGGCAGAGGTGAGAGAGA | TTGGCTGTCAGAAGAGAGCA | 57,58 |

### Statistical Analyses

Difference in nucleotide transition/transversion rates from next generation sequencing SNV calls were determined using the two-proportion Z test with Yates' correction for continuity. Significance of gender, location, metastasis, and individual genomic loci differences between molecular subgroups were analyzed using a two-sided Fisher's exact test. Kruskal-Wallis test was used to assess significance of tumor subgroups in relation to age, and counts of genomic alterations. Student's t test and Mann-Whitney-Wilcoxon test with FDR correction was used to test for differences in gene expression and methylation, respectively, between groups. All analyses were conducted in the R statistical environment (v2.15.2) or with SPSS version 22.0. A p value of <0.05 was regarded as significant for all analyses.
Table 3: List of genes for determining subgroup. To define core molecular and cellular features of ATRT sub-groups, transcriptional and methylation data were integrated which revealed that genes involved in lineage determination and developmental signaling were amongst the most highly differentially methylated and expressed loci across ATRT sub-groups. Group 1 ATRT are characterized by a neurogenic gene signature with genes involved in neural development (FABP7, ASCL1, MYCN, c1orf61, TUBB2B/3/4A, SEMA6A, TTYH1) and upregulation of Notch signaling (DLL1/3 HESS/6). The most commonly differentially expressed and methylated genes in group 2A and 2B tumors were enriched for functions in BMP signaling (BMP4, BAMBI, GDF5, FOXC1) and mesenchymal differentiation (SERPINF1, FBN2, MSX1, PDGFRβ, SOST, COLIA2/5A2). Group 2A tumors were further distinguished by enrichment of genes implicated in visual cortex/hind brain development (OTX2, CLDN1/3, CLIC6), tyrosine (TYR), and retinol (RBP1/7,RDH5/10) metabolism, whereas upregulation of MYC and HOXB/C clusters were characteristic of Group 2B tumors. Additionally, Ingenuity Pathway Analysis revealed that Group 2A tumors had functions in pluripotency and regulation of EMT, whereas Group 2B tumors exhibited more heterogeneous signaling profiles with enrichment of Interferon signaling, cell adhesion and cytoskeletal development genes. Methylation analyses revealed patterns of hypo- and hypermethylation at the promoters of functionally important developmental/cell lineage signaling genes, which also correlated with chromatin openness profiling determined by ATAC-seq analyses. In particular, hypomethylation and chromatin openness of a novel enhancer region of PDGFRβ correlated significantly with upregulation of PDGFRβ in group 2A and 2B tumors.

| | Diagnostic | Gene Bank |
|---|---|---|
| Gene | Group | Accession |
| FABP7 | Group 1 | NM_001446 |
| ASCL1 | Group 1 | NM_004316 |
| C1orf61 | Group 1 | NM_006365 |
| STMN4 | Group 1 | NM_030795 |
| POU3F2 | Group 1 | NM_005604 |
| HES5 | Group 1 | NM_001010926 |
| TTYH1 | Group 1 | NM_001005367 |
| SEMA6A | Group 1 | NM_001300780 |
| SOX21 | Group 1 | NM_007084 |
| DLL1 | Group 1 | NM_005618 |
| DLL3 | Group 1 | NM_203486 |
| MYCN | Group 1 | NM_001293233 |
| SOX2 | Group 1 | NM_003106 |
| HES6 | Group 1 | NM_018645 |
| PTPRZ1 | Group 1 | NM_002851 |
| KIF21A | Group 1 | NM_001173463 |
| BAI3 | Group 1 | NM_001704 |
| ELAVL3 | Group 1 | NM_032281 |
| ELAVL4 | Group 1 | NM_001144777 |
| NEUROG2 | Group 1 | NM_024019 |
| GNAO1 | Group 1 | NM_020988 |
| MEIS2 | Group 1 | NM_002399 |
| NNAT | Group 1 | NM_005386 |
| MT3 | Group 1 | NM_005954 |
| TUBB2B | Group 1 | NM_178012 |
| C1QL4 | Group 1 | NM_001008223 |
| GNG3 | Group 1 | NM_012202 |
| LIMCH1 | Group 1 | NM_001289124 |
| WSCD1 | Group 1 | NM_015253 |
| SYT4 | Group 1 | NM_020783 |
| STOX2 | Group 1 | NR_132761 |
| PNCK | Group 1 | NM_001039582 |
| GPM6B | Group 1 | NM_001001994 |
| GADD45G | Group 1 | NM_006705 |
| PDLIM3 | Group 1 | NM_001114107 |
| SCG5 | Group 1 | NM_003020 |
| KIF1A | Group 1 | NM_001244008 |
| PDGFRB | Group 2A and 2B | NM_002609 |
| MSX1 | Group 2A and 2B | NM_002448 |
| BAMBI | Group 2A and 2B | NM_012342 |
| BMP4 | Group 2A and 2B | NM_001202 |
| SERPINF1 | Group 2A and 2B | NM_002615 |
| CLIC6 | Group 2A | NM_001317009 |
| NOV | Group 2A | NM_002514 |
| OTX2 | Group 2A | NM_021728 |
| CLDN1 | Group 2A | NM_021101 |
| CLDN3 | Group 2A | NM_001306 |
| TYR | Group 2A | NM_000372 |
| SOST | Group 2A | NM_025237 |
| MSX2 | Group 2A | NM_002449 |
| LAMB1 | Group 2A | NM_002291 |
| NMB | Group 2A | NM_021077 |
| WNT5A | Group 2A | NM_003392 |
| CCK | Group 2A | NM_000729 |
| FLRT3 | Group 2A | NM_013281 |
| MAP | Group 2A | NM_005360 |
| ZIC4 | Group 2A | NM_001243256 |
| FZD9 | Group 2A | NM_003508 |
| TPD52L1 | Group 2A | NM_001300994 |
| PCP4 | Group 2A | NM_006198 |
| SLC13A4 | Group 2A | NM_012450 |
| ENPP2 | Group 2A | NM_001130863 |
| DDIT4L | Group 2A | NM_145244 |
| TTR | Group 2A | NM_000371 |
| CA2 | Group 2A | NM_000067 |
| CTSL2 | Group 2A | NM_001201575 |
| OCA2 | Group 2A | NM_001300984 |
| CPXM2 | Group 2A | NM_198148 |
| LOC440585 | Group 2A | NM_001101376 |
| TSPAN10 | Group 2A | NM_001290212 |
| TRPM3 | Group 2A | NM_001007471 |
| LOC653506 | Group 2A | NM_001004431 |
| CYB5R2 | Group 2A | NM_001302826 |
| BCMO1 | Group 2A | NM_017429 |
| COLEC12 | Group 2A | NM_130386 |
| IER3 | Group 2A | NM_003897 |
| FAM19A3 | Group 2A | NM_182759 |
| GDF5 | Group 2A and 2B | NM_000557 |
| COL1A2 | Group 2A and 2B | NM_000089 |
| COL5A2 | Group 2A and 2B | NM_000393 |
| MYC | Group 2A and 2B | NM_002467 |
| FOXC1 | Group 2A and 2B | NM_001453 |
| HOXC8 | Group 2B | NM_022658 |
| HOXA5 | Group 2B | NM_019102 |
| HOXC6 | Group 2B | NM_153693 |
| HOXB5 | Group 2B | NM_002147 |
| IGFBP6 | Group 2B | NM_002178 |
| HOXB8 | Group 2B | NM_024016 |
| HOXB2 | Group 2B | NM_002145 |
| HOXB7 | Group 2B | NM_004502 |
| THBS1 | Group 2B | NM_003246 |
| IGFBP4 | Group 2B | NM_001552 |
| H19 | Group 2B | NR_131224 |
| LUM | Group 2B | NM_002345 |
| CRABP2 | Group 2B | NM_001199723 |
| COL3A1 | Group 2B | NM_000090 |
| COL1A1 | Group 2B | NM_000088 |
| EMP3 | Group 2B | NM_001425 |
| COL6A3 | Group 2B | NM_004369 |
| BST2 | Group 2B | NM_004335 |
| CDC42EP5 | Group 2B | NM_145057 |
| DCN | Group 2B | NM_001920 |
| S100A4 | Group 2B | NM_019554 |
| PRRX2 | Group 2B | NM_016307 |
| MGP | Group 2B | NM_000900 |
| COL5A1 | Group 2B | NM_001278074 |
| S100A6 | Group 2B | NM_014624 |
| MXRA5 | Group 2B | NM_015419 |
| ACTG2 | Group 2B | NM_001615 |
| EMILIN2 | Group 2B | NM_032048 |
| S100A13 | Group 2B | NM_001024210 |
| TIMP1 | Group 2B | NM_003254 |
| TCEA3 | Group 2B | NM_003196 |

TABLE 4
Sequences in FIGS.

| FIG. | Sequence | NO |
|---|---|---|
| 1D | CATGAATGATTCA...AAGTTCTGA | 59...60 |
| 1E | TCTTGCATCTGGGCTCGGG | 61 |
| 10B | cgcctcccgggttcatgCCATTCTCCTGCCTCAG | 62 |
| 10D | CATGAATGATTCA...AAGTTCTGA | 59...60 |
| 10F | TCTTGCATCTGGGCTCGGG | 61 |
| 12B seq1 | GATCCAATAGCAGGAAAACAGAAGCTGTCGAAGAGGCTTCAAGCCATGC | 63 |
| | TATAATCG | |
| 12B seq1 | DPIAGKQKLSKRLQAML | 64 |
| (aa) | | |
| 12B seq2 | GGGTTTCACTATGTKGGCCAGGCATTGACTACCAGCTC | 65 |
| 12C seq1 | GAAGGAGTACATTGTCAGGGTGATAGTTAA | 66 |
| 12C seq1 | EGVHCQGDS | 67 |
| (aa) | | |
| 12C seq2 | GAAGGAGTACATTGTCAGGAAAACTTTTCTTTACTGACTACGGGAATGTC | 68...69 |
| | GC...ATGGGATGA | |
| 12C seq2 | EGVHCQENFSLLTTGMS..MG | 70 |
| (aa) | | |
| 12C seq3 | GAAGGAGTACATTGTCAGGATAAGAAACTGTGGTGGGCAGACCAAAACT | 71 |
| | TAGCCCAG | |
| 12C seq3 | EGVHCQDKKLWWADQNLAQ | 72 |
| (aa) | | |
| 12E seq1 | CCCAACTGTGTCTAAGACCTGGGGGTGGGAGGGA | 73 |
| 12E seq2 | TTCCCAACTGTGTCTAAGACCTGGGGGTGGGAGGGAAGCAGAT | 74 |

### Example 2

### Generating a Minimal Genetic Signature for ATRT Sub-Typing

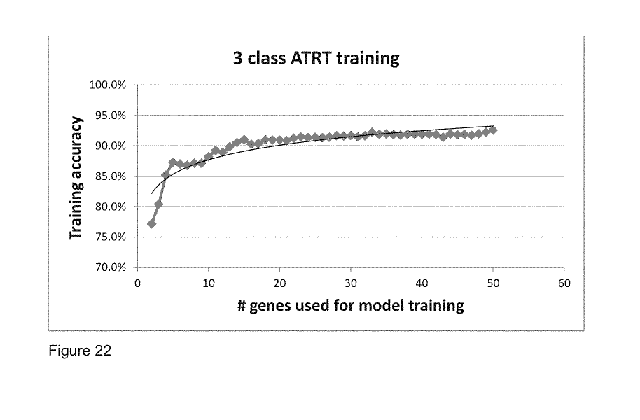

Signature genes for each ATRT subgroup were selected based on subgroup-specific gene expression using the Illumina HT-12 microarray. Student's t test corrected for false-discovery (FDR) was used to compare each subgroup to the remaining subgroup to identify the most highly significant, differentially expressed genes. It is expected that if a gene is upregulated for a particular subgroup, it is also likely hypomethylated. To test for the ability to classify ATRT samples using signature genes identified for each group, the Prediction Analysis for Microarray (PAM) algorithm was used to build a class prediction model. First, a cross-validation training set was constructed based on gene expression data to verify model learning. A dataset of 90 ATRTs with known subgroups were randomly partitioned into groups of 60 to be used as training sets for the prediction analysis, whereas the remaining 30 samples were defined the predictor set to test for model prediction accuracy using various numbers of genes (2-50 genes; FIG. 22). The procedure was repeated 1,000 times to assess the reproducibility and accuracy of the model for each gene set. Using this approach, a minimal gene set of 30 genes was identified which predicts ATRT subgroup with up to 95% accuracy (Table 5).

After demonstrating the capacity to train a classifier based on expression data, the gene sets may be used to construct a NanoString assay using the EpiTyper technology. Three housekeeping genes (ACTB, GAPDH, and LDHA) may be included in the gene set for biological normalization purposes. Probe sets for each gene may be designed and synthesized at nanoString Technologies. Raw nanoString counts for each gene may be normalized using the three housekeeping genes, and resulting data may be log2-transformed and then used as input for class prediction analysis. Prediction accuracy may be assessed as above to validate the utility as a generalizable model applicable for diagnosis of unknown ATRTs. All class prediction analyses were performed in the R statistical programming environment (v3.0.2).

TABLE 5
ATRT sub-typing panel

| Gene | Group Upregulated | RefSeq Accession |
|---|---|---|
| CLIC6 | Group 2A | NM_001317009 |
| FABP7 | Group 1 | NM_001446 |
| NNAT | Group 1 | NM_005386 |
| ASCL1 | Group 1 | NM_004316 |
| H19 | Group 2B | NR_131224 |
| MT3 | Group 1 | NM_005954 |
| C1orf61 | Group 1 | NM_006365 |
| PTPRZ1 | Group 1 | NM_002851 |
| CCK | Group 2A | NM_000729 |
| COL1A2 | Group 2A and 2B | NM_000089 |
| TPD52L1 | Group 2A | NM_001300994 |
| PCP4 | Group 2A | NM_006198 |
| SLC13A4 | Group 2A | NM_012450 |
| OTX2 | Group 2A | NM_021728 |
| LUM | Group 2B | NM_002345 |
| ENPP2 | Group 2A | NM_001130863 |
| TUBB2B | Group 1 | NM_178012 |
| DDIT4L | Group 2A | NM_145244 |
| STMN4 | Group 1 | NM_030795 |
| TTR | Group 2A | NM_000371 |
| POU3F2 | Group 1 | NM_005604 |
| CA2 | Group 2A | NM_000067 |
| HES5 | Group 1 | NM_001010926 |
| CLDN1 | Group 2A | NM_021101 |
| CTSL2 | Group 2A | NM_001201575 |
| C1QL4 | Group 1 | NM_001008223 |
| OCA2 | Group 2A | NM_001300984 |
| MSX1 | Group 2A and 2B | NM_002448 |
| GNG3 | Group 1 | NM_012202 |
| BAMBI | Group 2A and 2B | NM_012342 |

While the present disclosure has been described with reference to what are presently considered to be the examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### REFERENCES

Anthony, T. E., Klein, C., Fishell, G., and Heintz, N. (2004). Radial glia serve as neuronal progenitors in all regions of the central nervous system. Neuron 41, 881-890.
Araujo, J., and Logothetis, C. (2010). Dasatinib: a potent SRC inhibitor in clinical development for the treatment of solid tumors. Cancer Treat Rev 36, 492-500.
Biegel, J. A., Tan, L., Zhang, F., Wainwright, L., Russo, P., and Rorke, L. B. (2002). Alterations of the hSNF5/INI1 gene in central nervous system atypical teratoid/rhabdoid tumors and renal and extrarenal rhabdoid tumors. In Clin Cancer Res, pp. 3461-3467.
Birks, D. K., Donson, A. M., Patel, P. R., Dunham, C., Muscat, A., Algar, E. M., Ashley, D. M., Kleinschmidt-Demasters, B. K., Vibhakar, R., Handler, M. H., and Foreman, N. K. (2011). High expression of BMP pathway genes distinguishes a subset of atypical teratoid/rhabdoid tumors associated with shorter survival. In Neuro-oncology, pp. 1296-1307.
Bourdeaut, F., Lequin, D., Brugieres, L., Reynaud, S., Dufour, C., Doz, F., Andre, N., Stephan, J. L., Perel, Y., Oberlin, O., et al. (2011). Frequent hSNF5/INI1 germline mutations in patients with rhabdoid tumor. Clin Cancer Res 17, 31-38.
Buenrostro, J. D., Giresi, P. G., Zaba, L. C., Chang, H. Y., and Greenleaf, W. J. (2013). Transposition of native chromatin for fast and sensitive epigenomic profiling of open chromatin, DNA-binding proteins and nucleosome position. Nature methods 10, 1213-1218.
Cancer Genome Atlas Research, N. (2013). Genomic and epigenomic landscapes of adult de novo acute myeloid leukemia. N Engl J Med 368, 2059-2074.
Cecconi, F., Proetzel, G., Alvarez-Bolado, G., Jay, D., and Gruss, P. (1997). Expression of Meis2, a Knotted-related murine homeobox gene, indicates a role in the differentiation of the forebrain and the somitic mesoderm. Developmental dynamics: an official publication of the American Association of Anatomists 210, 184-190.
Chen, K., Wallis, J. W., Mclellan, M. D., Larson, D. E., Kalicki, J. M., Pohl, C. S., Mcgrath, S. D., Wendl, M. C., Zhang, Q., Locke, D. P., et al. (2009). BreakDancer: an algorithm for high-resolution mapping of genomic structural variation. In Nature Methods, pp. 677-681.
Chi, S. N., Zimmerman, M. A., Yao, X., Cohen, K. J., Burger, P., Biegel, J. A., Rorke-Adams, L. B., Fisher, M. J., Janss, A., Mazewski, C., et al. (2008). Intensive Multimodality Treatment for Children With Newly Diagnosed CNS Atypical Teratoid Rhabdoid Tumor. In J Clin Oncol, pp. 385-389.
Court, F., Baniol, M., Hagege, H., Petit, J. S., Lelay-Taha, M. N., Carbonell, F., Weber, M., Cathala, G., and Forne, T. (2011). Long-range chromatin interactions at the mouse Igf2/H19 locus reveal a novel paternally expressed long non-coding RNA. Nucleic Acids Res 39, 5893-5906.
Cowin, P. A., George, J., Fereday, S., Loehrer, E., Van Loo, P., Cullinane, C., Etemadmoghadam, D., Ftouni, S., Galletta, L., Anglesio, M. S., et al. (2012). LRP1B Deletion in High-Grade Serous Ovarian Cancers Is Associated with Acquired Chemotherapy Resistance to Liposomal Doxorubicin. In Cancer Res, pp. 4060-4073.
Eaton, K. W., Tooke, L. S., Wainwright, L. M., Judkins, A. R., and Biegel, J. A. (2011). Spectrum of SMARCB1/INI1 mutations in familial and sporadic rhabdoid tumors. In Pediatr Blood Cancer, pp. 7-15.
Ericson, J., Norlin, S., Jessell, T. M., and Edlund, T. (1998). Integrated FGF and BMP signaling controls the progression of progenitor cell differentiation and the emergence of pattern in the embryonic anterior pituitary. Development (Cambridge, England) 125, 1005-1015.
Feinberg, A. P., Ohlsson, R., and Henikoff, S. (2006). The epigenetic progenitor origin of human cancer. Nature Reviews Genetics 7, 21-33. Filippova, G. N., Fagerlie, S., Klenova, E. M., Myers, C., Dehner, Y., Goodwin, G., Neiman, P. E., Collins, S. J., and Lobanenkov, V. V. (1996). An exceptionally conserved transcriptional repressor, CTCF, employs different combinations of zinc fingers to bind diverged promoter sequences of avian and mammalian c-myc oncogenes. Molecular and cellular biology 16, 2802-2813.
Gadd, S., Sredni, S. T., Huang, C.-C., Perlman, E. J., and Group, Renal Tumor Committee of the Children's Oncology Group. (2010). Rhabdoid tumor: gene expression clues to pathogenesis and potential therapeutic targets. In Lab. Invest., pp. 724-738.
Geling, A., Steiner, H., Willem, M., Bally-Cuif, L., and Haass, C. (2002). A gamma-secretase inhibitor blocks Notch signaling in vivo and causes a severe neurogenic phenotype in zebrafish. In EMBO Rep., pp. 688-694.
Ginn, K. F., and Gajjar, A. (2012). Atypical teratoid rhabdoid tumor: current therapy and future directions. Front Oncol 2, 114.
Ha, G., Roth, A., Lai, D., Bashashati, A., Ding, J., Goya, R., Giuliany, R., Rosner, J., Oloumi, A., Shumansky, K., et al. (2012). Integrative analysis of genome-wide loss of heterozygosity and monoallelic expression at nucleotide resolution reveals disrupted pathways in triple-negative breast cancer. Genome Res 22, 1995-2007.
Hagege, H., Klous, P., Braem, C., Splinter, E., Dekker, J., Cathala, G., de Laat, W., and Forne, T. (2007). Quantitative analysis of chromosome conformation capture assays (3C-qPCR). Nat Protoc 2, 1722-1733.
Hasselblatt, M., Gesk, S., Oyen, F., Rossi, S., Viscardi, E., Giangaspero, F., Giannini, C., Judkins, A. R., Fruhwald, M. C., Obser, T., et al. (2011). Nonsense mutation and inactivation of SMARCA4 (BRG1) in an atypical teratoid/rhabdoid tumor showing retained SMARCB1 (INIl) expression. Am J Surg Pathol 35, 933-935.
Heinz, S., Benner, C., Spann, N., Bertolino, E., Lin, Y. C., Laslo, P., Cheng, J. X., Murre, C., Singh, H., and Glass, C. K. (2010). Simple combinations of lineage-determining transcription factors prime cis-regulatory elements required for macrophage and B cell identities. Molecular cell 38, 576-589.
Hilden, J. M. (2004). Central Nervous System Atypical Teratoid/Rhabdoid Tumor: Results of Therapy in Children Enrolled in a Registry. In J Clin Oncol, pp. 2877-2884.
Jackson, E. M., Sievert, A. J., Gai, X., Hakonarson, H., Judkins, A. R., Tooke, L., Perin, J. C., Xie, H., Shaikh, T. H., and Biegel, J. A. (2009). Genomic analysis using high-density single nucleotide polymorphism-based oligonucleotide arrays and multiplex ligation-dependent probe amplification provides a comprehensive analysis of INI1/SMARCB1 in malignant rhabdoid tumors. In Clin Cancer Res, pp. 1923-1930.
Johnson, W. E., Li, C., and Rabinovic, A. (2007). Adjusting batch effects in microarray expression data using empirical Bayes methods. Biostatistics 8, 118-127.
Kaartinen, V., Gonzalez-Gomez, I., Voncken, J. W., Haataja, L., Faure, E., Nagy, A., Groffen, J., and Heisterkamp, N. (2001). Abnormal function of astroglia lacking Abr and Bcr RacGAPs. Development 128, 4217-4227.
Kadoch, C., and Crabtree, G. R. (2015). Mammalian SWI/SNF chromatin remodeling complexes and cancer: Mechanistic insights gained from human genomics. Sci Adv 1, e1500447.
Kantarjian, H., Giles, F., Wunderle, L., Bhalla, K., O′Brien, S., Wassmann, B., Tanaka, C., Manley, P., Rae, P., Mietlowski, W., et al. (2006). Nilotinib in imatinib-resistant CML and Philadelphia chromosome-positive ALL. N Engl J Med 354, 2542-2551.
Kao, C.-L., Chiou, S.-H., Chen, Y.-J., Singh, S., Lin, H.-T., Liu, R.-S., Lo, C.-W., Yang, C.-C., Chi, C.-W., Lee, C.-h., and Wong, T.-T. (2005). Increased expression of osteopontin gene in atypical teratoid/rhabdoid tumor of the central nervous system. In Modern Pathology, pp. 769-778.
Klochendler-Yeivin, A., Fiette, L., Barra, J., Muchardt, C., Babinet, C., and Yaniv, M. (2000). The murine SNF5/INI1 chromatin remodeling factor is essential for embryonic development and tumor suppression. EMBO reports 1, 500-506.
Knutson, S. K., Warholic, N. M., Wigle, T. J., Klaus, C. R., Allain, C. J., Raimondi, A., Porter Scott, M., Chesworth, R., Moyer, M. P., Copeland, R. A., et al. (2013). Durable tumor regression in genetically altered malignant rhabdoid tumors by inhibition of methyltransferase EZH2. In Proc Natl Acad Sci USA, pp. 7922-7927.
Lafay-Cousin, L., Hawkins, C., Carret, A. S., Johnston, D., Zelcer, S., Wilson, B., Jabado, N., Scheinemann, K., Eisenstat, D., Fryer, C., et al. (2012). Central nervous system atypical teratoid rhabdoid tumours: the Canadian Paediatric Brain Tumour Consortium experience. In Eur J Cancer, pp. 353-359.
Lonze, B. E., Riccio, A., Cohen, S., and Ginty, D. D. (2002). Apoptosis, axonal growth defects, and degeneration of peripheral neurons in mice lacking CREB. In Neuron, pp. 371-385.
Louis, D. N., and Wiestler, O. D. (2007). WHO Classification of Tumours of the Central Nervous System. ed 4th. IARC; Lyon.
Malik, A. N., Vierbuchen, T., Hemberg, M., Rubin, A. A., Ling, E., Couch, C. H., Stroud, H., Spiegel, I., Farh, K. K., Harmin, D. A., and Greenberg, M. E. (2014). Genome-wide identification and characterization of functional neuronal activity-dependent enhancers. Nat Neurosci 17, 1330-1339.
Morin, R. D., Mungall, K., Pleasance, E., Mungall, A. J., Goya, R., Huff, R. D., Scott, D. W., Ding, J., Roth, A., Chiu, R., et al. (2013). Mutational and structural analysis of diffuse large B-cell lymphoma using whole-genome sequencing. Blood 122, 1256-1265.
Morris, T. J., Butcher, L. M., Feber, A., Teschendorff, A. E., Chakravarthy, A. R., Wojdacz, T. K., and Beck, S. (2014). ChAMP: 450 k Chip Analysis Methylation Pipeline. Bioinformatics 30, 428-430.
Mozzetta, C., Pontis, J., Fritsch, L., Robin, P., Portoso, M., Proux, C., Margueron, R., and Ait-Si-Ali, S. (2014). The histone H3 lysine 9 methyltransferases G9a and GLP regulate polycomb repressive complex 2-mediated gene silencing. Molecular cell 53, 277-289.
Nelson, B. R., Hartman, B. H., Ray, C. A., Hayashi, T., Bermingham-McDonogh, O., and Reh, T. A. (2009). Acheate-scute like 1 (Ascii) is required for normal delta-like (DII) gene expression and notch signaling during retinal development. Developmental dynamics: an official publication of the American Association of Anatomists 238, 2163-2178.
Rix, U., Hantschel, O., Durnberger, G., Remsing Rix, L. L., Planyaysky, M., Fernbach, N. V., Kaupe, I., Bennett, K. L., Valent, P., Colinge, J., et al. (2007). Chemical proteomic profiles of the BCR-ABL inhibitors imatinib, nilotinib, and dasatinib reveal novel kinase and nonkinase targets. Blood 110, 4055-4063.
Roberts, C. W., Leroux, M. M., Fleming, M. D., and Orkin, S. H. (2002). Highly penetrant, rapid tumorigenesis through conditional inversion of the tumor suppressor gene Snf5. Cancer cell 2, 415-425.
Roberts, C. W., and Orkin, S. H. (2004). The SWI/SNF complex—chromatin and cancer. Nature reviews Cancer 4, 133-142.
Robertson, G., Schein, J., Chiu, R., Corbett, R., Field, M., Jackman, S. D., Mungall, K., Lee, S., Okada, H. M., Qian, J. Q., et al. (2010). De novo assembly and analysis of RNA-seq data. Nature methods 7, 909-912.
Ross-Innes, C. S., Stark, R., Teschendorff, A. E., Holmes, K. A., Ali, H. R., Dunning, M. J., Brown, G. D., Gojis, O., Ellis, I. O., Green, A. R., et al. (2012). Differential oestrogen receptor binding is associated with clinical outcome in breast cancer. Nature 481, 389-393.
Roy, A., Gonzalez-Gomez, M., Pierani, A., Meyer, G., and Tole, S. (2013). Lhx2 Regulates the Development of the Forebrain Hem System. Cereb Cortex.
Schlemmer, M., Schinwald, N., Bruns, C., Berger, F., and Reichardt, P. (2012). Response to nilotinib as a first-line treatment for metastatic gastrointestinal stromal tumors. J Gastrointest Cancer 43, 385-387.
Sévenet, N., Sheridan, E., Amram, D., Schneider, P., Handgretinger, R., and Delattre, O. (1999). Constitutional mutations of the hSNF5/INI1 gene predispose to a variety of cancers. In Am J Hum Genet, pp. 1342-1348.
Sharifi, K., Morihiro, Y., Maekawa, M., Yasumoto, Y., Hoshi, H., Adachi, Y., Sawada, T., Tokuda, N., Kondo, H., Yoshikawa, T., et al. (2011). FABP7 expression in normal and stab-injured brain cortex and its role in astrocyte proliferation. Histochemistry and cell biology 136, 501-513.
Sommer, A., Bousset, K., Kremmer, E., Austen, M., and Luscher, B. (1998). Identification and characterization of specific DNA-binding complexes containing members of the Myc/Max/Mad network of transcriptional regulators. The Journal of biological chemistry 273, 6632-6642.
Sonoda, I., Imoto, I., Inoue, J., Shibata, T., Shimada, Y., Chin, K., Imamura, M., Amagasa, T., Gray, J. W., Hirohashi, S., and Inazawa, J. (2004). Frequent silencing of low density lipoprotein receptor-related protein 1B (LRP1 B) expression by genetic and epigenetic mechanisms in esophageal squamous cell carcinoma. In Cancer Res, pp. 3741-3747.
Tang, Y., Gholamin, S., Schubert, S., Willardson, M. I., Lee, A., Bandopadhayay, P., Bergthold, G., Masoud, S., Nguyen, B., Vue, N., et al. (2014). Epigenetic targeting of Hedgehog pathway transcriptional output through BET bromodomain inhibition. Nature medicine 20, 732-740.
Tekautz, T. M. (2005). Atypical Teratoid/Rhabdoid Tumors (ATRT): Improved Survival in Children 3 Years of Age and Older With Radiation Therapy and High-Dose Alkylator-Based Chemotherapy. In J Clin Oncol, pp. 1491-1499.
Torchia, J., Picard, D., Lafay-Cousin, L., Hawkins, C. E., Kim, S. K., Letourneau, L., Ra, Y. S., Ho, K. C., Chan, T. S., Sin-Chan, P., et al. (2015). Molecular subgroups of atypical teratoid rhabdoid tumours in children: an integrated genomic and clinicopathological analysis. The lancet oncology 16, 569-582.
Trapnell, C., Roberts, A., Goff, L., Pertea, G., Kim, D., Kelley, D. R., Pimentel, H., Salzberg, S. L., Rinn, J. L., and Pachter, L. (2012). Differential gene and transcript expression analysis of RNA-seq experiments with TopHat and Cufflinks. Nat Protoc 7, 562-578.
Versteege, I., Sévenet, N., Lange, J., Rousseau-Merck, M. F., Ambros, P., Handgretinger, R., Aurias, A., and Delattre, O. (1998). Truncating mutations of hSNF5/INI1 in aggressive paediatric cancer. In Nature, pp. 203-206.
Wang, J., Mullighan, C. G., Easton, J., Roberts, S., Heatley, S. L., Ma, J., Rusch, M. C., Chen, K., Harris, C. C., Ding, L., et al. (2011). CREST maps somatic structural variation in cancer genomes with base-pair resolution. In Nature Methods, pp. 652-654.
Wilkerson, M. D., and Hayes, D. N. (2010). ConsensusClusterPlus: a class discovery tool with confidence assessments and item tracking. Bioinformatics 26, 1572-1573.
Wilson, B. G., Helming, K. C., Wang, X., Kim, Y., Vazquez, F., Jagani, Z., Hahn, W. C., and Roberts, C. W. (2014). Residual complexes containing SMARCA2 (BRM) underlie the oncogenic drive of SMARCA4 (BRG1) mutation. Molecular and cellular biology 34, 1136-1144.
Ye, K., Schulz, M. H., Long, Q., Apweiler, R., and Ning, Z. (2009). Pindel: a pattern growth approach to detect break points of large deletions and medium sized insertions from paired-end short reads. In Bioinformatics, pp. 2865-2871.
Yu, P. B., Hong, C. C., Sachidanandan, C., Babitt, J. L., Deng, D. Y., Hoyng, S. A., Lin, H. Y., Bloch, K. D., and Peterson, R. T. (2008). Dorsomorphin inhibits BMP signals required for embryogenesis and iron metabolism. In Nat. Chem. Biol., pp. 33-41.
Zerbino, D. R., and Birney, E. (2008). Velvet: algorithms for de novo short read assembly using de Bruijn graphs. In Genome Res, pp. 821-829.
Zhang, M., Fang, H., Zhou, J., and Herring, B. P. (2007). A Novel Role of Brg1 in the Regulation of SRF/MRTFA-dependent Smooth Muscle-specific Gene Expression. In J Biol Chem, pp. 25708-25716.
Zhang, X., Cowper-Sal lari, R., Bailey, S. D., Moore, J. H., and Lupien, M. (2012). Integrative functional genomics identifies an enhancer looping to the SOX9 gene disrupted by the 17q24.3 prostate cancer risk locus. Genome Res 22, 1437-1446.
Zhang, Y., Liu, T., Meyer, C. A., Eeckhoute, J., Johnson, D. S., Bernstein, B. E., Nusbaum, C., Myers, R. M., Brown, M., Li, W., and Liu, X. S. (2008). Model-based analysis of ChIP-Seq (MACS). Genome Biol 9, R137.

1. A method of treating a subject with a Group 1 type rhabdoid tumor comprising administering an inhibitor that targets Notch signaling or targets an epigenetic regulator selected from a BET domain protein, G9a or EZH2 to the subject in need thereof.

2. The method of claim 1, wherein the inhibitor that targets Notch signaling is DAPT.

3. The method of claim 1, wherein the inhibitor that targets a BET domain protein is JQ1.

4. The method of claim 1, wherein the inhibitor that targets G9a is UNC0368.

5. The method of claim 1, wherein the inhibitor that targets EZH2 is UNC1999.

6. The method of claim 1, wherein the Group 1 rhabdoid tumor is an ATRT CNS tumor.

7. The method of claim 1, wherein the Group 1 rhabdoid tumor is a non-CNS tumor.

8. The method of claim 1, wherein the subject is identified as Group 1 based on genomic features, epigenomic features and/or transcriptional features.

9. The method of claim 8, wherein the epigenomic features comprise hypomethylation of the genes listed in Table 3 or Table 5 for Group 1 subjects and/or wherein the transcriptional features comprise increased level of expression of the genes listed in Table 3 or Table 5 for Group 1 subjects.

10. A method of treating a subject with a Group 2 type rhabdoid tumor comprising administering an inhibitor that targets BMP or PDGFRβ signaling to a subject in need thereof.

11. The method of claim 10, wherein the inhibitor that targets BMP is dorsomorphin.

12. The method of claim 10, wherein the inhibitor that targets PDGFRβ is dasatinib or nilotinib.

13. The method of claim 10, wherein the Group 2 rhabdoid tumor is an ATRT CNS tumor.

14. The method of claim 10, wherein the Group 2 rhabdoid tumor is a non-CNS tumor.

15. The method of claim 10, wherein the subject is identified as Group 2 based on genomic features, epigenomic features and/or transcriptional features.

16. The method of claim 15, wherein the epigenomic features comprise hypomethylation of the genes listed in Table 3 or Table 5 for Group 2, Group 2A and/or Group 2B subjects or wherein the transcriptional features comprise increased level of expression of the genes listed in Table 3 or Table 5 for Group 2, Group 2A and/or Group 2B subjects.

17. A method of determining the type of rhabdoid tumor of a sample comprising:
a) (i) determining a sample gene expression profile and/or (ii) a sample methylation profile from DNA from the sample, said sample gene expression profile and/or sample methylation profile comprising the level of gene expression and/or methylation, respectively, of at least three, optionally at least 5, at least 7, at least 10, at least 15, at least 20 or all of the genes listed in Table 3 or Table 5;
b) determining the level of similarity of said sample gene expression profile and/or sample methylation profile to one or more control profiles, wherein
(i) a high level of similarity of the sample profile to a Group 1 specific control profile; a low level of similarity to a Group 2A or Group 2B control profile indicates that the sample is a Group 1 rhabdoid tumor; or a higher level of similarity to a Group 1 specific control profile than to a Group 2A or Group 2B control profile indicates the sample is a Group 1 type rhabdoid tumor;
(ii) a high level of similarity of the sample profile to a Group 2A specific control profile; a low level of similarity to a Group 2B or Group 1 control profile indicates that the sample is a Group 2A rhabdoid tumor; or a higher level of similarity to a Group 2A specific control profile than to a Group 2B or Group 1 control profile indicates the sample is a Group 2A type rhabdoid tumor; or
(iii) a high level of similarity of the sample profile to a Group 2B specific control profile; a low level of similarity to a Group 2A or Group 1 control profile indicates that the sample is a Group 2B rhabdoid tumor; or a higher level of similarity to a Group 2B specific control profile than to a Group 2A or Group 1 control profile indicates the sample is a Group 2B type rhabdoid tumor.

18. The method of claim 17, wherein a) comprises (i) determining the sample gene expression profile and (ii) determining the sample methylation profile.

19. The method of claim 17, wherein determining the sample methylation profile in a)ii) comprises the steps:
a) providing the sample comprising genomic DNA from the subject;
b) optionally, isolating DNA from the sample;
c) optionally, treating DNA from the sample with bisulfite for a time and under conditions sufficient to convert non-methylated cytosines to uracils;
d) optionally, amplifying the DNA; and
e) determining the methylation status at the selected genes by means of bisulfite sequencing, pyrosequencing, methylation-sensitive single-strand conformation analysis (MS-SSCA), high resolution melting analysis (HRM), combined bisulfite restriction analysis (COBRA), methylation-sensitive single nucleotide primer extension (MS-SnuPE), base-specific cleavage/MALDI-TOF, methylation-specific PCR (MSP), methylation-sensitive restriction enzyme-based methods, microarray-based methods, whole-genome bisulfite sequencing (WGBS, MethylC-seq or BS-seq), reduced-representation bisulfite sequencing(RRBS), and/or enrichment-based methods such as MeDIP-seq, MBD-seq, or MRE-seq.

20. The method of claim 17, wherein the sample is derived from tumor tissue or blood.

21. The method of claim 17, further comprising treating the subject with an inhibitor of notch or an inhibitor of an epigenomic regulator if the sample is typed as a Group 1 rhabdoid tumor.

22. The method of claim 17, further comprising treating the subject with an inhibitor of BMP or PDGFRβ signaling if the sample is typed as a Group 2A or Group 2B rhabdoid tumor.
